(19) United States
(12) Patent Application Publication
(10) Pub. No.: US 2023/0193219 A1
(43) Pub. Date: Jun. 22, 2023

(54) SUPEROXIDE DISMUTASE COMPOSITIONS AND METHODS

(71) Applicant: Blue Sky Innovation, LLC, Pleasant Grove, UT (US)

(72) Inventors: Danny C. Purser, Provo, UT (US); Stephen N. Pitcher, Alpine, UT (US)

(73) Assignee: Blue Sky Innovation, LLC, Pleasant Grove, UT (US)

(21) Appl. No.: 17/978,019

(22) Filed: Oct. 31, 2022

Related U.S. Application Data
(63) Continuation-in-part of application No. PCT/US2021/030355, filed on Apr. 30, 2021.
(60) Provisional application No. 63/018,488, filed on Apr. 30, 2020.

(51) Int. Cl. C12N 9/02; A61K 47/36; A61K 47/44; A61K 47/69; A61K 31/167; A61P 15/10; A61P 17/00; A61P 19/00; A61P 25/00

(52) U.S. Cl. CPC C12N 9/0091; A61K 31/167; A61K 47/36; A61K 47/44; A61K 47/6905; A61P 15/10; A61P 17/00; A61P 19/00; A61P 25/00

(57) ABSTRACT

The present disclosure is drawn to superoxide dismutase (SOD) compositions and methods thereof. A topical composition can comprise a combination of a therapeutically effective amount of superoxide dismutase (SOD) with a stabilizing carrier that is suitable for topical administration. A method of treating a condition in a subject that is responsive to treatment with superoxide dismutase (SOD) can comprise administering a therapeutically effective amount of the topical composition. A method of stabilizing a superoxide dismutase (SOD) composition can comprise combining an amount of SOD with deoxygenated water to form an SOD solution, and minimizing exposure of the SOD solution to reactive oxygen species (ROS).

### PRIORITY DATA

This application is a continuation-in-part of International Patent Cooperation Treaty Application No. PCT/US2021/030355, filed Apr. 30, 2021, which claims the benefit of United States Provisional Application Serial Number 63/018,488, filed Apr. 30, 2020, each of which is incorporated herein by reference in its entirety.

### FIELD OF THE DISCLOSURE

The present disclosure relates to compositions and dosage forms containing superoxide dismutase (SOD) as well as associated methods. Accordingly, this disclosure involves the fields of chemistry, pharmaceutical sciences, medicine, and other health sciences.

### BACKGROUND

Superoxide dismutase (SOD) is an enzyme that can catalyze the partitioning of the superoxide (O_{2^{–}}) radical into oxygen (O₂) and hydrogen peroxide (H₂O₂). Elevated levels of superoxide radicals can damage cells. In humans, there are three forms of superoxide dismutase: SOD1 located in the cytoplasm, nuclei, lysosomes, and peroxisomes; SOD2 located in the mitochondria, and SOD3 located in the vascular extracellular space. Therefore, SOD can regulate levels of free radicals in various locations in and around cells. Reactive oxygen species can interfere with SOD during manufacturing, storage, and administration. Accordingly, in view of the potential therapeutic benefits offered by SOD, compositions and methods of administration that place a maximum amount of SOD at a target treatment situs continue to be sought.

### SUMMARY OF THE DISCLOSURE

The present disclosure is drawn to pharmaceutical compositions and dosage forms containing superoxide dismutase (SOD) and related methods. In one embodiment, a superoxide dismutase composition is provided. The composition can comprise a combination of a therapeutically effective amount of superoxide dismutase (SOD) with a stabilizing carrier. In one embodiment, the stabilizing carrier can comprise deoxygenated water. In some examples, the deoxygenated water can have less than about 5 ppm of oxygen. The composition can further comprise a stabilizing agent. The stabilizing agent can encapsulate the SOD or otherwise form a stabilizing complex with the SOD.

In an additional embodiment, an SOD delivery system is provided. The SOD delivery system can include a composition as recited herein and a container configured to minimize an amount of reactive oxygen species (ROS) therein. The container can be further configured to minimize the amount of ROS in a headspace of the container and/or to minimize the entry of ROS into the container.

In still a further embodiment, a method of treating a human subject responsive to treatment with SOD is provided. In one embodiment, such a method can include administering a therapeutically effective amount of a composition or dosage form as recited herein to a subject.

In a yet further embodiment, a method of manufacturing a superoxide dismutase composition is provided. The method can comprise combining a superoxide dismutase (SOD) (e.g. as a powder) with deoxygenated water to form an SOD solution, and minimizing exposure of the SOD solution to reactive oxygen species (ROS). In one aspect, minimizing exposure of the SOD solution to the ROS can include minimizing an amount of the ROS in a headspace of a container that is containing or housing the SOD solution. In some examples, the method can further comprise combining the superoxide dismutase solution with a stabilizing agent.

In yet another embodiment, a method of stabilizing superoxide dismutase (SOD) is provided. The method can comprise combining an amount of SOD with deoxygenated water to form an SOD solution, and minimizing exposure of the SOD solution to ROS.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Features and advantages of the disclosure will be apparent from the detailed description which follows, taken in conjunction with the accompanying drawings, which together illustrate, by way of example, features of the disclosure; and wherein:

FIG. 1a depicts a method of manufacturing a stabilized superoxide dismutase composition in accordance with an example embodiment.

FIG. 1b depicts a method of stabilizing a superoxide dismutase composition in accordance with an example embodiment.

FIG. 2 shows SOD activity testing results in which a darker color represents decreasing activity in accordance with an example embodiment.

FIG. 3 shows liposome particle size results in accordance with an example embodiment.

FIG. 4 shows liposomal glutathione zeta potential results in accordance with an example embodiment.

Reference will now be made to the exemplary embodiments illustrated, and specific language will be used herein to describe the same. It will nevertheless be understood that no limitation of the scope of the disclosure is thereby intended.

### DESCRIPTION OF EMBODIMENTS

Although the following detailed description contains many specifics for the purpose of illustration, a person of ordinary skill in the art will appreciate that many variations and alterations to the following details can be made and are considered to be included herein. As such, the following embodiments are set forth without any loss of generality to, and without imposing limitations upon, any claims set forth. It is also to be understood that the terminology used herein is for the purpose of describing particular embodiments only, and is not intended to be limiting. Unless defined otherwise, all technical and scientific terms used have the same meaning as commonly understood by one of ordinary skill in the art to which this disclosure belongs.

### Definitions

It should be noted that, the singular forms “a,” “an,” and, “the” include plural referents unless the context clearly dictates otherwise. Thus, for example, reference to “an excipient” includes reference to one or more of such excipients, and reference to “the carrier” includes reference to one or more of such carriers.

As is known in the art, the term “superoxide dismutase” or “SOD” generally refers to an enzyme that alternately catalyzes the partitioning of the superoxide radical into oxygen (O₂) and hydrogen peroxide (H₂O₂). “SOD” can refer to human recombinant SOD (rhSOD), SOD1, SOD2, SOD3, or any combination thereof. Furthermore, in this written description, SOD provides specific individual support for human recombinant SOD (rhSOD), SOD1, SOD2, and SODS as though each were expressly recited.

As used herein, the terms “treat,” “treatment,” or “treating” refers to administration of a therapeutic agent to subjects who are either asymptomatic or symptomatic. In other words, “treat,” “treatment,” or “treating” can be to reduce, ameliorate, modulate, or eliminate symptoms associated with a condition present in a subject, or can be prophylactic, (i.e., to prevent or reduce the occurrence of the symptoms in a subject). Such prophylactic treatment can also be referred to as prevention of the condition.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be an SOD compound. The terms “additional active agent,” “supplemental active agent,” “secondary active agent,” and the like can be used interchangeably and refer to a compound, molecule, or material other than SOD that has physiologic activity when administered to a subject in an effective amount.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients. Furthermore, the term “dosage form” can include one or more formulation(s) or composition(s) provided in a format (e.g. a specific form, shape, vehicle, etc.) for administration to a subject. For example, an “oral dosage form” can be suitable for administration to a subject’s mouth. A “topical dosage form” can be suitable for administration to a subject’s skin by rubbing, etc.

As used herein, the term “stabilizing agent” refers to a compound, molecule, or substance that has a stabilizing effect on SOD or an additional active agent and increases the stability (e.g. reduces the degradation of reactivity of SOD) while in a composition or dosage form, or upon administration to a subject.

As used herein, the term “reactive form” refers to a compound, molecule, or substance that can reduce, oxidize, or reduce and oxidize another compound, molecule, or substance. A “reactive form of SOD” can refer to SOD that can reduce, oxidize, or reduce and oxidize superoxide.

As used herein, the term “fatty acid” refers to unionized carboxylic acids with a long aliphatic tail (chain), either saturated or unsaturated, conjugated or non-conjugated.

As used herein, the term “soluble” is a measure or characteristic of a substance or agent with regards to its ability to dissolve in a given solvent. The solubility of SOD in a particular component of the composition refers to the amount of the SOD dissolved to form a visibly clear solution at a specified temperature such as about 25° C. or about 37° C.

As used herein, the term “lipophilic,” refers to compounds that are not freely soluble in water; and the term “lipophilic surfactant” refers to surfactants that have HLB values of about 10 or less. Conversely, the term “hydrophilic” refers to compounds that are soluble in water; and the term “hydrophilic surfactant” refers to surfactants that have HLB values of more than about 10.

As used herein, the term “capsule fill” refers to the composition disposed in a capsule shell to provide a capsule dosage form.

As used herein, a “subject” refers to an animal. In one aspect the animal may be a mammal. In another aspect, the mammal may be a human.

A used herein, a “responder” is a subject who responds positively to SOD treatment or therapy. “Responder analysis” is the assessment of the effectiveness of SOD therapy in a subject, or a group of subjects, deemed to benefit from an SOD therapy.

As used herein, “group” or “group of subjects” refers to a collection subjects (e.g. at least 10 human subjects) who receive and respond to administration of the compositions disclosed herein, namely SOD compositions. In one aspect, the group can include at least 20 or at least 50 subjects. In another aspect, the group can include at least 100 subjects.

The term “oral administration” represents any method of administration in which an active agent can be administered by swallowing, chewing, or sucking of the dosage form. Oral administration can be intended for enteral delivery of an active agent or transmucosal delivery of the active agent. In some embodiments, the present compositions can be admixed with food or drink prior to being orally consumed.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total daily dose of from about 15 mcg/1 mL to about 1500 mcg/1 mL administered in two divided doses at least 12 hours apart (e.g. once with breakfast and once with dinner) with meals repeated daily for 30 days.

As used herein, “daily dose” refers to the amount of active agent (e.g. SOD) administered to a subject over a 24-hour period of time. The daily dose can be administered two or more administrations during the 24-hour period. In one embodiment, the daily dose provides for two administrations in a 24-hour period. With this in mind, an “initial dose” or “nitial daily dose” refers to a dose administered during the initial regimen or period of a dosing regimen.

As used herein, “non-liquid” when used to refer to the state of a composition disclosed herein refers to the physical state of the composition as being a semi-solid or solid.

As used herein, “solid” and “semi-solid” refers to the physical state of a composition that supports its own weight at standard temperature and pressure and has adequate viscosity or structure to not freely flow. Semi-solid materials may conform to the shape of a container under applied pressure.

As used herein, “titration” or “dose titration” or “dose adjustment” are used interchangeably and refer to an increase or decrease of the total daily dose of SOD administered to a subject, typically based on the response of the subject to the exogenously administered SOD. The dose can be increased or decreased based on the measurement of serum SOD concentration after a steady state has been achieved.

As used herein, “steady state” refers to the achievement of stable serum total SOD levels upon a continuous dosing regimen (e.g. once daily, twice daily etc.) of the administered SOD at a given dose, after at least 7 consecutive days (typically achieved after at least 15 days), following the start of the dosing regimen. Unless otherwise stated, steady states values set forth herein refer to steady states achieved after a final dose titration (i.e., no additional titrations are required), including situations where no dose titration is required. Similarly, as used herein, the “steady state serum concentration (Cₛₛ, Css)” or “mean steady state serum concentration (mean Cₛₛ)” of SOD refers to the achievement of a stable serum total SOD concentration in a subject or group of subjects, respectively, in response to a continuous dosing regimen (e.g., once daily, twice daily etc.) of the administered SOD at a given dose, after at least 7 days (typically achieved after at least 15 days), following the start of the dosing regimen. It should be further noted that when a dose adjustment (increase or decrease in total daily dose of SOD administered) is made as part of the dose-titration during the treatment, the mean Cₛₛ is achieved at least about 7 days after the initiation of the change in the dose administered.

As used herein, the terms “release” and “release rate” are used interchangeably to refer to the discharge or liberation of a substance, including without limitation a drug, from the dosage form into a surrounding environment such as an aqueous medium either in vitro or in vivo.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, the term “delayed release” refers to the release into an aqueous solution of the SOD from the composition or oral dosage form in a time delayed manner attributed either to the inherent nature of the composition or to a coating which may surround the composition or the oral dosage form. In one embodiment, the delayed release is such that about 20% or less of the SOD is released within the first 15 minutes after the composition is contacted by the aqueous solution.

The terms “serum SOD levels,” “plasma SOD concentration,” “SOD concentration in the blood,” and “serum SOD concentration,” are used interchangeably and refer to the “total” SOD concentration which is the sum of the bioavailable SOD including free and bound (if any) SOD concentrations. Unless otherwise specified, these values are “observed SOD concentrations without adjusting or correcting for the base-line serum SOD levels in the subject(s). As with any bio-analytical measure, for increased consistency the method employed to measure initial serum SOD levels should be consistent with the method used to monitor and re-measure serum SOD levels during clinical testing and SOD therapy for a subject. Unless otherwise stated, “SOD concentration” refers to serum total SOD concentration.

As used herein, the average serum SOD concentration can be determined using methods and practices known in the art. For example, the average baseline plasma SOD concentration of a subject can be the arithmetic mean of the total plasma SOD concentrations determined on at least two consecutive time points that are reasonably spaced from each other, for example from about 1 hour to about 168 hours apart. In a particular case, the plasma SOD concentration can be determined on at least two consecutive times that are about 12 hours to about 48 hours apart. In another particular method, the plasma SOD concentration of the subject can be determined at a time between about 5 o′clock and about 11 o′clock in the morning. Further, the plasma SOD concentration can be determined by standard analytical procedures and methods available in the art, such as for example, automated or manual immunoassay methods, liquid chromatography or liquid chromatography- tandem mass spectrometry (LC-MSMS) etc.

As used herein “single unit” when used to describe dosing of a subject refers to the dosage form being a single dosage form, e.g., a single tablet, capsule, pump or squirt of gel or solution, etc. In contrast, “multiple unit” when used to describe dosing of a subject refers to the dosage including two or more dosage forms, e.g. 2 tablets, 3 capsules, 2-4 pumps or squirts, etc. It is noteworthy that multiple unit dosage forms generally will be the same type of dosage forms (i.e., tablet or capsule) but are not required to be the same dosage form type.

As used herein, a “treatment situs” refers to a location on or within a subject where treatment is desired. For example, when treating a burn or a lesion, the treatment situs can be the area of the burn or lesion. When treating a respiratory condition affecting the lungs, the treatment situs can be the lungs. Further, as used herein, an “application situs” refers to a location on or in a subject where treatment is administered. For example, the application situs for an oral dosage formulation may be the subject’s mouth. Further, the application situs for an infusion dosage formulation may be an area where the infusion equipment enters the subject’s circulatory system. Yet further, the application situs for a topical dosage formulation may be the area of skin or mucosa to which the topical dosage formulation is applied. In some embodiments, the application situs may be substantially the same as the treatment situs (e.g. the composition or formulation is administered directly to the treatment site). In other embodiments, the application situs may be different from (e.g. distal from) the treatment situs. In such cases despite the fact that administration may be distal from the treatment situs, the composition or formulation still exerts a therapeutic effect at the treatment situs.

As used herein, “topical composition” or “topical administration” and the like refer to a composition suitable for administration directly to a skin or mucosa surface and from which an effective amount of a drug is released. In some embodiments, topical compositions can provide a local or localized therapeutic effect (e.g. at or near an application situs). For example, a topical composition when applied to a wound, a lesion, a burn, a canker sore, etc. (e.g. a treatment situs), may primarily exert a therapeutic effect at or around the application situs, but not substantially beyond it. In other embodiments, a topical composition can provide a regional effect. For example, a topical composition administered to a skin surface on a region of the body, such as a finger, arm, ankle, joint, etc. can exert a therapeutic effect within the region, but not substantially beyond. For example, a topical composition administered to the region of an ankle can have a therapeutic effect in and around the ankle, by for example, reducing edema, joint inflammation, pain, etc. In other embodiments, topical compositions can provide a systemic effect. In some aspects, a topical composition can provide the therapeutic effect though a mechanism of action where the drug or active agent itself arrives at the treatment situs. In other aspects, the topical composition can provide the therapeutic effect through an intermediate mechanism of action, such as biochemical cascade event, such as an enzymatic cascade or other signaling (e.g. cellular signaling, or inter/intra cellular signaling) event which ultimately exerts the desired therapeutic effect at the treatment situs. In some examples, such intermediate mechanism can allow treatment of a treatment situs that is distal from an application situs. In yet other examples, when treatment of a distal treatment situs occurs, the active agent may travel through dermal and other tissues from the application situs to the treatment situs and exert a direct effect.

As used herein, “transdermal” refers to the route of administration of a therapeutic agent through a skin surface when administered to the skin surface. When transdermally administered, the drug or active agent migrates from the application situs to a treatment situs and exerts a therapeutic effect. Transdermal compositions and dosage forms can include structures and/or devices which assist in holding the composition on a skin surface, such as, for example, backing films, adhesives, reservoirs, etc. Furthermore, transdermal compositions can include agents which aid or otherwise facilitate movement of the active agent from an application situs to a treatment situs (e.g. through the skin and into the subject’s circulatory system) such as penetration or permeation enhancers. Such penetration or permeation enhancers can also be used with topical formulations in some embodiments.

The term “skin” or “skin surface” includes not only the outer skin of a subject comprising one or more epidermal layers, but also mucosal surfaces such as the mucosa of the respiratory (including nasal and pulmonary), oral (mouth and buccal), vaginal, and rectal cavities. Hence, the term “transdermal” may encompass “transmucosal” as well.

As used herein, “co-administering” a first therapeutic agent with a second therapeutic agent can include concomitant administration within a suitable time window. In one example, the suitable time window can be less than one or more of: 1 hour, 45 minutes, 30 minutes, 15 minutes, 5 minutes, 2 minutes, 1 minute, or combinations thereof. Concomitant administration can be from the same composition or from different compositions.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

In this disclosure, “comprises,” “comprising,” “containing” and “having” and the like can have the meaning ascribed to them in U.S. Patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. Patent law. “Consisting essentially of” or “consists essentially of” have the meaning generally ascribed to them by U.S. Patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “maximized,” “minimized,” and the like refer to a property of a device, component, composition, or activity that is measurably different from other devices, components, compositions or activities that are in a surrounding or adjacent area, that are similarly situated, that are in a single device or composition or in multiple comparable devices or compositions, that are in a group or class, that are in multiple groups or classes, or as compared to the known state of the art. For example, a composition that has “increased” stability of SOD keeps a higher amount of its SOD content in a reduced form (SOD) rather than converting to an oxidized form of SOD for the same or greater amount of time as compared to a similar composition which does not achieve such results.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used. Occurrences of the phrase “in one embodiment,” or “in one aspect,” herein do not necessarily all refer to the same embodiment or aspect.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility to a numerical range endpoint by providing that a given value may be “a little above” or “a little below” the endpoint. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

Reference in this specification may be made to devices, structures, systems, or methods that provide “improved” performance. It is to be understood that unless otherwise stated, such “improvement” is a measure of a benefit obtained based on a comparison to devices, structures, systems or methods in the prior art. Furthermore, it is to be understood that the degree of improved performance may vary between disclosed embodiments and that no equality or consistency in the amount, degree, or realization of improved performance is to be assumed as universally applicable.

### EXAMPLE EMBODIMENTS

An initial overview of disclosure embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Superoxide dismutase (SOD) is an enzyme that can catalyze the partitioning of the superoxide (O_{2⁻}) radical into oxygen (O₂) and hydrogen peroxide (H₂O₂). The reaction can take place by: (a) Cu²⁺-SOD + O₂⁻ → Cu⁺-SOD + O₂, and (b) Cu⁺-SOD + O_{2⁻}+ 2H⁺ → Cu²⁺-SOD + H₂O₂. The resultant H₂O₂ can be degraded by catalase. There are three types of superoxide dismutase based on the metal cofactor: (1) the Cu/Zn type; (2) the Fe and Mn types, and (3) the Ni type. There are three forms of superoxide dismutase in humans: SOD1, SOD2, and SOD3. SOD1 and SOD3 contain copper and zinc, and SOD2 contains manganese. Another form of SOD is human recombinant SOD (rhSOD).

Superoxide is an ROS that can cause damage in the cell. The reaction of superoxide with SOD is a first-order reaction. Dysfunction in SOD can play a role in various diseases. For example, amyotrophic lateral sclerosis (ALS) can be caused by mutations in the SOD1 enzyme. SOD also plays a role in the slowing of conditions associated with aging. Accordingly, in view of the potential therapeutic benefits offered by SOD, compositions and methods of administration that place a maximum amount of SOD at a target treatment situs continue to be sought.

In one embodiment, a superoxide dismutase composition can comprise a combination of a therapeutically effective amount of superoxide dismutase (SOD) with a stabilizing carrier. In one aspect, the stabilizing carrier can comprise deoxygenated water.

In an additional embodiment, an SOD delivery system is provided. The SOD delivery system can include a composition as recited herein and a container configured to minimize an amount of reactive oxygen species (ROS) therein. The container can be further configured to minimize the amount of ROS in a headspace of the container and/or to minimize the entry of ROS into the container.

In still a further embodiment, a method of treating a human subject responsive to treatment with SOD is provided. In one embodiment, such a method can include administering a therapeutically effective amount of a composition or dosage form as recited herein to a subject.

In a yet further embodiment, a method of manufacturing a superoxide dismutase composition is provided. The method can comprise combining a superoxide dismutase (SOD) powder with deoxygenated water to form an SOD solution, and minimizing exposure of the SOD solution to reactive oxygen species (ROS).

In yet another embodiment, a method of stabilizing superoxide dismutase (SOD) is provided. The method can comprise combining an amount of SOD with deoxygenated water to form an SOD solution, and minimizing exposure of the SOD solution to ROS.

The present disclosure further includes topical compositions and related methods for administering SOD. In one embodiment, such a topical composition can comprise a combination of a therapeutically effective amount of superoxide dismutase (SOD) with a stabilizing carrier that is suitable for topical administration. A method of treating a condition in a subject that is responsive to treatment with superoxide dismutase (SOD) can comprise administering a therapeutically effective amount of the topical composition. A method of stabilizing a superoxide dismutase (SOD) composition can comprise combining an amount of SOD with deoxygenated water to form an SOD solution, and minimizing exposure of the SOD solution to reactive oxygen species (ROS).

### Compositions

With this in mind, in one embodiment of the present disclosure, a superoxide dismutase composition can comprise a combination of a therapeutically effective amount of superoxide dismutase (SOD) with a stabilizing carrier. In some aspects, the stabilizing carrier can comprise deoxygenated water. In some examples, the deoxygenated water can have less than one or more of about 1 ppm, 2 ppm, 4 ppm, 6 ppm, or 8 ppm of oxygen. In some examples, the deoxygenated water can have less than about 10 ppm of oxygen.

In a further embodiment, the composition can further comprise a support agent selected from the group consisting of a stabilizing agent, a preservative, an emollient, an adjuvant, the like, and combinations thereof. Further, the support agent can be a stabilizing agent. The stabilizing agent can encapsulate the SOD or form a stabilizing complex with the SOD.

In another embodiment, the stabilizing agent can be an oxygen scavenger, a lipid, a synthetic polymer, a natural polymer, a polyelectrolyte, a protein, an amino acid, a co-polymer, an emulsion, a gel, an inert gas, or a combination thereof. In some aspects, the stabilizing agent can be an encapsulating agent that forms a nanoemulsion, a liposome, a nanogel, or a combination thereof. In some aspects, the encapsulating agent can be selected from the group consisting essentially of: chitosan, cyclodextrin, dextran, starch, silicon, tragacanth, and combinations thereof. In some aspects, the stabilizing agent can form a stabilizing complex with the SOD.

In some aspects, the stabilizing agent can be a lipid selected from the group consisting essentially of: phospholipids, glycolipids, cholesterol, triglycerides, fatty acids, fatty acid glycerides, surfactants, or a combination thereof. In a further aspect, the lipid can form a liposome, an inverted micelle, or a combination thereof.

In one embodiment, the composition can further comprise an additional active agent selected from the group consisting of: catalase, an antioxidant, an anti-infective agent, an antibiotic, an anti-tumor agent, an anti-inflammatory agent, an analgesic, an anesthetic, an anti-rheumatic agent, a growth factor, a cytokine, an amino acid, a protein, a vaccine, a hormone, a vitamin, the like, and combinations thereof.

In some embodiments, the composition can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof. In one aspect, the composition can comprise a transdermal dosage form, a topical dosage form, an oral dosage form, a parenteral dosage form, a nebulizer dosage form, a transmucosal dosage form, and combinations thereof.

In the aforementioned composition, the SOD can be present in the composition at a concentration of from about 0.0001 wt% to about 10 wt%. In one example, the SOD can be present in the composition at a concentration of from about 0.0001 wt% to about 1 wt%. In another example, the SOD can be present in the composition at a concentration of from about 0.001 wt% to about 1 wt%. In one more example, the SOD can be present in the composition at a concentration of from about 0.01 wt% to about 0.1 wt%. In some examples, the SOD can be present in the composition at a concentration of from about 0.005 wt% to about 0.05 wt%.

In one aspect, the SOD can remain in a reactive form after a period of about 365 days when stored at 75% relative humidity and room temperature. In another aspect, the SOD can remain in a reactive form after a period of about 6 months when stored at 75% relative humidity and room temperature. In another aspect, the SOD can remain in a reactive form after a period of about 3 months. In another aspect, the SOD can remain in a reactive form after a period of about 4 weeks when stored at 75% relative humidity and 40° C. In another aspect, the SOD can remain in a reactive form after a period of about 2 weeks when stored at 50-75% relative humidity and 40° C.

In yet other embodiments, specific dosage forms for delivering an effective amount of SOD and/or providing SOD therapy are provided. In some aspects, such dosage forms can include an effective (e.g. therapeutically effective) amount of SOD in combination with a stabilizing carrier that is effective in maximizing the amount of SOD that remains in a reduced form. In some aspects, the carrier can comprise deoxygenated water. In other embodiments, the carrier can be substantially free of one or more of a-D-glucopyranoside units. In some embodiments, the dosage forms can have the same or similar basic chemical and/or physical properties as discussed herein with respect to the compositions, namely, materials, ingredients, components, amounts, concentrations, etc. while being specifically prepared for specific types or routes of administration.

More specifically, the discussion herein relating to compositional components that can be used in the pharmaceutical compositions is also equally applicable to the dosage form embodiments and related methods disclosed herein unless expressly stated to the contrary. It is also noteworthy that the discussion relating to compositional components that can be used in the pharmaceutical compositions is also equally applicable to the dosage form embodiments and related methods disclosed herein unless expressly stated to the contrary. Thus, for example, teachings regarding the use of stabilizing agents for use in the pharmaceutical compositions disclosed herein are also equally applicable to the dosage forms and related methods described herein and vice versa.

Further, in addition to SOD, the pharmaceutical compositions and dosage forms can further include a support agent selected from the group consisting of: a stabilizing agent, a carrier, a preservative, an emollient, an adjuvant, the like, and combinations thereof.

In one aspect, the support agent can be a stabilizing agent. In one embodiment, the stabilizing agent can comprise about 0.5% to 50% by weight of the pharmaceutical composition or dosage form. In another embodiment, the stabilizing agent can comprise about 2% to 50%, or 10-50% or 15-50% or 10-35% or 10-30% or 10-25% or 15-25% or 15-30% or 20-30% or 20-25%, or more specifically, about 35%, about 30%, about 25%, about 20%, about 18%, about 16%, about 15%, about 12%, about 10%, about 8%, about 6%, about 4%, or about 2% by weight composition, of the pharmaceutical composition or dosage form.

The stabilizing agent can include an oxygen scavenger, a lipid, a synthetic polymer, a natural polymer, a protein, an amino acid, a co-polymer, an inert gas, or a combination thereof.

In some examples, the stabilizing agent can include an oxygen scavenger. Non-limiting examples of oxygen scavengers can include, but are not limited to, suitable combinations of: a transition metal catalyst (e.g., copper), ascorbic acid, glucose oxidase, catalase, polyunsaturated fatty-acids, soybean oil, sesame oil, cottonseed oil, squalene, fatty acids, polybutadiene, yeast, antioxidants, the like, and combinations thereof. In some examples, the stabilizing agent can include a synthetic polymer. In some examples, the stabilizing agent can include a natural a-D-glucopyranoside polymer, or a non-a-D-glucopyranoside polymer.

In another aspect, the stabilizing agent can be a lipid selected from the group consisting essentially of: phospholipids, glycolipids, cholesterol, triglycerides, fatty acids, fatty acid glycerides, surfactants, or a combination thereof.

In another example, the lipid can form a liposome, an inverted micelle, or a combination thereof. Non-limiting examples of liposomes can include, but are not limited to, the multilamellar vesicle (MLV), the small unilamellar liposome vesicle (SUV), the large unilamellar vesicle (LUV), the cochleate vesicle, and combinations thereof.

In another example, the lipid can form an inverted micelle which can include nanometer-sized (1-10 nm) water droplets dispersed in a non-polar solvent obtained by the action of surfactants. Non-limiting examples of surfactants can include, but are not limited to, AOT [sodium bis(2-ethyl hexyl) sulfosuccinate], CTAB (cetyltrimethylammonium bromide), dode-cyl penta(oxyethylene) ether (C12E5), n-dodecyloctaoxyethylene glycol monoether (C12E8), cetylbenzyldimethylammonium chloride (CBAC), didodecyl-dimethylammonium bromide (DDAB), sorbitan monooleate, and sodium dodecylbenzene-sulfonate (NaDBS), Triton X-100[polyoxyethylene(10)isooctylphenyl ether], poly-oxyethylene(4) lauryl ether (known as Brij30), pentaoxyethylene-glycol-nonyl-phenyl ether (known as Igepal-CO520), poly(oxyethylene)5nonylphenol ether (NP-5), poly(oxyethylene)9nonylphenol ether (NP-9), poly(oxyethylene)12 nonylphenol ether (NP-12), and combinations thereof.

In some embodiments, a stabilizing carrier can comprise water. In one aspect, the water can be deoxygenated. In one specific embodiment, the deoxygenated water has less than about 5 ppm of oxygen. In another specific embodiment, the deoxygenated water has less than about 3 ppm of oxygen. In another specific embodiment, the deoxygenated water has less than about 1 ppm of oxygen. In one specific embodiment, the deoxygenated water has less than about 0.5 ppm of oxygen. In another specific embodiment, the deoxygenated water has less than about 0.1 ppm of oxygen. Generally, the oxygen content of the water is referred to as “oxygen” and determined by known techniques such as oximetry.

In one embodiment, the composition can be a topical composition. The topical composition can comprise a combination of a therapeutically effective amount of superoxide dismutase (SOD) with a stabilizing carrier that is suitable for topical administration. In one aspect, the stabilizing carrier can comprise deoxygenated water. In one aspect, the deoxygenated water can have less than one or more of: about 10 ppm of oxygen, about 5 ppm of oxygen, about 3 ppm of oxygen, about 1 ppm of oxygen, or combinations thereof.

The SOD can be present in the topical composition at a concentration of from about 0.0001 wt% to about 10 wt%. In one example, the SOD can be present in the topical composition at a concentration of from about 0.0001 wt% to about 1 wt%. In another example, the SOD can be present in the topical composition at a concentration of from about 0.001 wt% to about 1 wt%. In one more example, the SOD can be present in the topical composition at a concentration of from about 0.01 wt% to about 0.1 wt%. In some examples, the SOD can be present in the topical composition at a concentration of from about 0.005 wt% to about 0.05 wt%.

In one aspect, the topical composition can consist essentially of SOD-1 and deoxygenated water. In another aspect, the topical composition can consist essentially of SOD-2 and deoxygenated water. In another aspect, the topical composition can consist essentially of SOD-3 and deoxygenated water.

In another aspect, the topical composition can be substantially free of ethanol, capsaicin, menthol, carbomer, the like, and combinations thereof. The capsaicin, menthol carbomer, and ethanol (e.g,. greater than 5 wt%) can be detrimental for the storage and long-term stability of the SOD composition when stored for a selected storage time at ambient humidity and temperature. In some cases, a storage time of greater than one or more of 1 day, 1 week, 2 weeks, 4 weeks, 3 months, 6 months, one year, or combinations thereof can be detrimental to SOD activity in these examples.

In one more aspect, the SOD can comprise human recombinant SOD (rhSOD), SOD-1, SOD-2, SOD-3, or a combination thereof.

In one aspect, the topical composition can include a support agent selected from the group consisting of: a stabilizing agent, a preservative, an emollient, an adjuvant, the like, and combinations thereof. In one example, the support agent can be a stabilizing agent. In one example, the stabilizing agent can include an oxygen scavenger, a lipid, a synthetic polymer, a natural polymer, a polyelectrolyte, a protein, an amino acid, a co-polymer, an emulsion, a gel, an inert gas, the like, or a combination thereof. In another example, the stabilizing agent can be an encapsulating agent that forms a nanoemulsion, a liposome, a nanogel, the like, or a combination thereof. In one aspect, the encapsulating agent can be selected from the group consisting essentially of: chitosan, cyclodextrin, dextran, starch, silicon, tragacanth, the like, and combinations thereof.

In one aspect, the stabilizing agent can form a stabilizing complex with the SOD. In another aspect, the stabilizing agent can be a lipid selected from the group consisting essentially of: phospholipids, glycolipids, cholesterol, triglycerides, fatty acids, fatty acid glycerides, surfactants, the like, or a combination thereof. In another aspect, the lipid can form a liposome, an inverted micelle, or a combination thereof.

In some examples, the topical composition can include an additional active agent. The additional active agent can be a member selected from the group consisting of: a catalase, an antioxidant, an anti-infective agent, an antibiotic, an anti-tumor agent, an anti-inflammatory agent, an anesthetic, an analgesic, an anti-rheumatic agent, a growth factor, a cytokine, an amino acid, a protein, a vaccine, a hormone, a vitamin, the like, and combinations thereof.

In one example, the additional active agent can be an anesthetic. Non-limiting examples of anesthetics include a local anesthetic of the amino amide type, the like, or combinations thereof. In one example, the anesthetic can be a member selected from the group consisting of: articaine, bupivacaine, cinchocaine, etidocaine, levobupivacaine, lidocaine, mepivacaine, prilocaine, ropivacaine, trimecaine, and combinations thereof. The additional active agent can be in the same or a different composition as the SOD. In one example, the anesthetic can be lidocaine.

The additional active agent can be present at various amounts. In one aspect, the additional active can be present at a concentration of from about 0.0001 wt% to about 10 wt%. In another aspect, the additional active can be present at a concentration of from about 0.0001 wt% to about 1 wt%. In one more aspect, the additional active can be present at a concentration of from about 0.001 wt% to about 1 wt%. In yet one more aspect, the additional active can be present at a concentration of from about 0.1 wt% to about 1 wt%.

The topical composition can have any suitable pH for topical application. In one example, the topical composition can have a pH of from about 6.8 to about 7.4. In another example, the topical composition can have a pH of from about 7.0 to about 7.2.

The topical composition can have any suitable viscosity for applying a topical substance. In one example, the topical composition can have a viscosity of from about 1 cP to about 15 cP. In another example, the topical composition can have a viscosity of from about 20 cP to about 100 cP. In one more example, the topical composition can have a viscosity of from about 300 cP to about 1000 cP.

The topical composition can be formulated in many ways. In one aspect, the topical composition can be formulated as one of: a solution, a suspension, an emulsion, a gel, a hydrogel, a thermo-responsive gel, a cream, an ointment, a paste, an adhesive, a liquid reservoir, a patch, the like, or a combination thereof.

In one aspect, when the SOD is present in the topical composition at a concentration of 0.02 wt%: (a) more than about 80% of the SOD can remain in a reactive form after a period of about 2 weeks when stored at room temperature and 75% relative humidity, or (b) more than about 80% of the SOD can remain in a reactive form after a period of about 2 weeks when stored at 40° C. and 75% relative humidity, or (c) more than about 80% of the SOD can remain in a reactive form after a period of about 4 weeks when stored at room temperature and 75% relative humidity, or (d) more than about 80% of the SOD can remain in a reactive form after a period of about 4 weeks when stored at 40° C. and 75% relative humidity.

In addition to the SOD, the pharmaceutical compositions and dosage forms can further include at least one additional pharmaceutically active agent or can be formulated to be co-administered with other active agents in order to treat a target condition.

Non-limiting examples of additional active agents that can be included with or co-administered with the pharmaceutical composition or dosage form include: catalase, an antioxidant, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, the like, and combinations thereof. Such compounds could be co-administered with the compositions and dosage forms of the present disclosure in order to provide improved clinical outcomes.

Antioxidants can be included in the present compositions and dosage forms for their effect as an additional active agent or in order to help in stabilizing the SOD. Various antioxidants can be used including, but not limited to: N-acetyl-cysteine, hydroxytyrosol (HXT), reduced glutathione (rGSH), catalase, Vitamin A, Vitamin C, Vitamin E, coenzyme Q10, managanese, iodide, melatonin, alpha-carotene, astaxanthin, beta-carotene, canthaxanthin, cryptoxanthin, lutein, lycopene, zeaxanthin, apigenin, luteolin, tangeritin, isorhamnetin, kaempferol, myricetin, proanthocyanidins, quercetin, eriodyctiol, hesperetin, naringenin, catechin, gallocatechin, epicatechin, epigallocatechin, theaflavin, thearubigins, daidzein, genistein, glycitein, resveratrol, pterostilbene, cyanidin, delphinidin, malvidin, pelargonidin, peonidin, petunidin, chicoric acid, cholorogenic acid, cinnamic acid, ellagic acid, ellagitannins, gallic acid, gallotannins, rosmarinic acid, slaicyclic acid, curcumin, flavonolignans, xanthones, eugenol, capsaicin, bilirubin, citric acid, oxalic acid, phytic acid, R-alpha-Lipoic acid, the like, and combinations thereof.

The additional active agent can include one or more anti-infective agents, namely an agent that can kill or prevent an infectious organism (e.g., a pathogen) from spreading. Thus, anti-infective agents can include antibacterial agents, antifungal agents, antiviral agents, antiprotozoan agents, the like, or combinations thereof. Non-limiting examples can include amebicides such as chloroquine, nitazoxanide, paromomycin, tinidazole, metronidazole, iodoquinole, or the like; aminoglycosides such as tobramycin, gentamicin, amikacin, kanamycin, neomycin, streptomycin, or the like; anthelmintics such as albendazole, ivermectin, praziquantel, pyrantel, mebendazole, miltefosine, niclosamide, piperazine, thiabendazole, or the like; antifungals such as itraconazole, posaconazole, ketoconazole, fluconazole, clotrimazole, isavuconazole, miconazole, voriconazole, echinocandins, terbinafine, griseofulvin, flucytosine, nystatin, amphotericin b, or the like; antimalarials such as chloroquine, quinine, hydroxychloroquine, mefloquine, primaquine, pyrimethamine, halofantrine, doxycycline, or the like; antituberculosis agents such as aminosalicylic acid, bedaquiline, isoniazid, ethambutol, pyrazinamide, ethionamide, rifampin, rifabutin, rifapentine, capreomycin, cycloserine, streptomycin, or the like; antivirals such as amantadine, rimantadine, ritonavir, cobicistat, peginterferon alfa-2a, peginterferon alfa 2b, maraviroc, raltegravir, dolutegravir, elvitegravir, sofosbuvir, enfuvirtide, fomivirsen, foscarnet, oseltamivir, zanamivir, peramivir, etravirine, efavirenz, nevirapine, delavirdine, rilpivirine, daclatasvir, adefovir, entecavir, telbivudine, didanosine, tenofovir, abacavir, lamivudine, zidovudine, stavudine, emtricitabine, zalcitabine, boceprevir, simeprevir, fosamprenavir, lopinavir, darunavir, telaprevir, ritonavir, tipranavir, atazanavir, nelfinavir, amprenavir, indinavir, saquinavir, ganciclovir, valacyclovir, famciclovir, acyclovir, valganciclovir, ribavirin, cidofovir, or the like; carbapenems such as doripenem, meropenem, cilastatin, ertapenem, or the like; cephalosporins such as avibactam, ceftolozane, ceftazidime, tazobactam, cefadroxil, cephalexin, cefazolin, ceftaroline, loracarbef, cefotetan, cefuroxime, cefprozil, cefaclor, cefoxitin, ceftibuten, cefotaxime, ceftriaxone, cefpodoxime, cefixime, cefdinir, defditoren, ceftazidime, ceftizoxime, cefepime, or the like; glycopeptide antibiotics such as vancomycin, dalbavancin, oritavancin, telavancin, or the like; glycocyclines such as tigecycline, or the like; leprostatics such as thalidomide, dapsone, clofazimine, or the like; lincomycin, or the like; clindamycin, or the like; ketolides such as telithromycin, or the like; macrolides such as azithromycin, fidaxomicin, erythromycin, clarithromycin, or the like; antibiotics such as aztreonam, daptomycin, chloramphenicol, colistimethate, fosfomycin, rifaximin, metronidazole, sulfamethoxazole, atovaquone, bacitracin, dalfopristin, erythromycin, furazolidone, pentamidine, polymyxin b, spectinomycin, trimetrexate, linezolid, tedizolid, penicillins (e.g. ampicillin, amoxicillin, carbenicillin, piperacillin, ticarcillin, nafcillin, dicloxacillin, cloxacillin, oxacillin, or the like), quinolones (e.g. lomefloxacin, norfloxacin, ofloxacin, gatifloxacin, moxifloxacin, ciprofloxacin, levofloxacin, gemifloxacin, cinoxacin, nalidixic acid, sparfloxacin, or the like), sulfonamides (e.g. sulfamethoxazole, sulfadiazine, sulfisoxazole, or the like), tetracyclines (e.g. tetracycline, demeclocycline, doxycycline, minocycline, or the like), or the like; urinary anti-infectives such as methenamine, methylene blue, fosfomycin, nitrofurantoin, trimethoprim, cinoxacin, nalidixic acid, oxytetracycline, or the like; hydrates thereof, acids thereof, bases thereof, salts thereof, or combinations of any of such anti-infective agents.

In some examples, the additional active agent can also include any suitable antitumor agent. Non-limiting examples of antitumor agents can include angiogenesis inhibitors such as angiostatin k1-3, angiostatin k1-5, DL-a-difluoromethylomithine, endostatin, fumagillin, genistein, minocycline, staurosporine, (+/-)-thalidomide, or the like; DNA intercalators such as bleomycin, carboplatin, carmustine, chlorambucil, cisplatin, cyclophosphamide, cis-diammineplatinum(II) dichloride, melphalan, mitoxantrone, oxaliplatin, or the like; DNA synthesis inhibitors such as (+/-)-amethopterin, 3-amino-1,2,4-benzotraizine-1,4-dioxide, aminopterin, cytosine β-D-arabinofuranoside, 5-fluoro-5′-deoxyuridine, 5-fluorouracil, ganciclovir, hydroxyurea, mitomycin C, or the like; transcriptioin regulators such as actinomycin D, daunorubicin, doxorubicin, homoharringtonine, idarubicin, or the like; enzyme inhibitors such as S(+)-camptothecin, curcumin, (-)-deguelin, 5,6-dichlorobenzimidazole 1-β-D-ribofuranoside, etoposide, formestane, fostriecin, hispidin, 2-imino-1-imidazolidineacetic acid, mevinolin, trichostatin A, tyrphostin AG 34, tyrphostin AG 879, or the like; gene regulation agents such as 5-aza-2′-deoxycytidine, 5-azacytidine, cholecalciferol, 4-hydroxytamoxifen, melatonin, mifepristone, raloxifene, vitamin A aldehyde, vitamin A acid, vitamin A, 9-cis-retinoic acid, 13-cis-retinoic acid, tamoxifen, troglitazone, or the like; microtubule inhibitors such as colchicine, docetaxel, dolastatin 15, etoposide, irinotecan, nocodazole, paclitaxel, podophyllotoxin, rhizoxin, vinblastine, vincristine, vindesine, vinorelbine, or the like; other antitumor agents such as 17-(allylamino)-17-demethoxygeldanamycin, 4-amino-1,8-naphthalimide, apigenin, brefeldin A, cimetidine, dichloromethylene-diphosphonic acid, leuprolide, luteinizing hormone-releasing hormone, pifithrin-α, rapamycin, sex hormone-binding globulin, thapsigargin, bikunin, ifosfamide, temozolomide, capecitabine, methotrexate, gemcitabine, pemetrexed, mitomycin, epirubicin, bevacizumab, cetuximab, gefitinib, imatinib, trastuzamab, denosumab, rituximab, sunitinib, zoledronate, abiraterone, anastrozole, bicalutamide, exemestane, goserelin, medroxyprogesterone, octreotide, tamoxifen, bendamustine, lomustine, procarbazine, streptozocin, fludarabine, raltitrexed, mitoxantrone, eribulin, topotecan, afatinib, aflibercept, BCG, crizotinib, dabrafenib, interferon, ipilimumab, lapatinib, nivolumab, panitumumab, pembrolizumab, pertuzumab, sorafenib, trastuzumab emtansine, temsirolimus, vemurafenib, ibandronic acid, pamidronate, bexarotene, buserelin, cyproterone, degarelix, folinic acid, fulvestrant, lanreotide, lenalidomide, letrozole, leuprorelin, megestrol, mesna, thalidomide, or the like; hydrates thereof, acids thereof, bases thereof, salts thereof, or combinations of any of such antitumor agents.

In some examples, the additional active agent can also include any suitable anti-inflammatory agent. Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

In some examples, the additional active agent can also include any suitable analgesic. Non-limiting examples of analgesics can include anti-inflammatory agents, such as those listed above, acetaminophen, codeine, dihydrocodeine, tramadol, meperidine, hydrocodone, oxycodone, morphine, fentanyl, hydromorphone, buprenorphine, methadone, diamorphine, pethidine, histamines and salts thereof, such as histamine dihydrochloride, the like, hydrates thereof, acids thereof, bases thereof, or salts thereof, or combinations thereof.

In some examples, the additional active agent can also include any suitable anesthetic. Non-limiting examples of anesthetics can include articaine, bupivacaine, cinchocaine, etidocaine, levobupivacaine, lidocaine, mepivacaine, prilocaine, ropivacaine, trimecaine, the like, or combinations thereof.

In some examples, the additional active agent can also include any suitable anti-rheumatic agent. Non-limiting examples of anti-rheumatic agents can include methotrexate, sulfasalazine, chloroquine, hydroxychloroquine, leflunomide, azathioprine, cyclosporine, minocycline, abatacept, rituximab, tocilizumab, anakinra, adalimumab, etanercept, infliximab, cetolizumab, golimumab, D-penicillamine, auranofin, the like, hydrates thereof, acids thereof, bases thereof, or salts thereof, or combinations thereof.

In some examples, the additional active agent can also include any suitable growth factor. Non-limiting examples of supplementary growth factors can include platelet-derived growth factor (PDGF), epidermal growth factor (EGF), transforming growth factor- alpha (TGF-α), fibroblast growth factor (FGF), nerve growth factor (NGF), erythropoietin, transforming growth factor-beta (TGF-β), insulin-like growth factor-1 (IGF-1), insulin-like growth factor-2 (IGF-2), the like, or combinations thereof.

In some examples, the additional active agent can also include any suitable supplementary cytokine. Non-limiting examples of supplementary cytokines can include interleukins, lymphokines, monokines, interferons, colony stimulating factors, chemokines, the like, or combinations thereof.

In some examples, the additional active agent can also include any suitable amino acid. Non-limiting examples can include arginine, histidine, isoleucine, leucine, lysine, methionine, phenylalanine, tryptophan, valine, acetyl-L-carinitine arginate, alpha-aminoadipic acid, alpha-amino-N-butyric acid, beta-alanine, beta-amino-isobutyric acid, carnosine, citruline, gamma-amino butyric acid, hydroxyproline, 1-methylhistidine, 3-methylhistidine, N-acytyl-L-cysteine, omithine, para-aminobenzoic acid, phosphoserine, phosphoethanolamine, taurine, the like, isomers thereof, hydrates thereof, salts thereof, acids thereof, bases thereof, or any combinations thereof.

In some examples, the additional active agent can also include any suitable protein. Non-limiting examples can include cytokines and/or growth factors, such as those listed above, as well as antibodies, Fc-fusion proteins, anticoagulants, blood factors, bone morphogenetic proteins, engineered protein scaffolds, enzymes, hormones, thrombolytics, the like, or combinations thereof.

In some examples, the additional active agent can also include a vaccine. Non-limiting examples of vaccines can include adenovirus vaccine, coronavirus, coxsackie B vaccine, cytomegalovirus vaccine, dengue vaccine, Eastern equine encephalitis vaccine, ebola vaccine, enterovirus vaccine, Epstein-barr vaccine, hepatitis A vaccine, hepatitis B vaccine, hepatitis C vaccine, hepatitis E vaccine, HIV vaccine, human papillomavirus vaccine, HTLV-1 T-lymphotrophic vaccine, influenza vaccine, Japanese encephalitis vaccine, Marburg vaccine, measles vaccine, mumps vaccine, norovirus vaccine, polio vaccine, rabies vaccine, respiratory syncytial virus (RSV) vaccine, rotavirus vaccine, rubella vaccine, severe acute respiratory syndrome (SARS) vaccine, varicella vaccine, smallpox vaccine, West Nile virus vaccine, yellow fever vaccine, anthrax vaccine, DPT vaccine, Q fever vaccine, Hib vaccine, tuberculosis vaccine, meningococcal vaccine, typhoid vaccine, pneumococcal vaccine, cholera vaccine, caries vaccine, ehrlichiosis vaccine, leprosy vaccine, lyme disease vaccine, staphylococcus aureus vaccine, streptococcus pyogenes vaccine, syphilis vaccine, tularemia vaccine, Yersinia pestis vaccine, the like, or combinations thereof.

In some examples, the additional active agent can also include a hormone. Non-limiting examples of hormones can include progestogens, androgens, estrogens, somatostatins, growth hormones, thyroid hormones, glucocorticoids, the like, or combinations thereof.

In some examples, the additional active agent can also include a vitamin. Non-limiting vitamins can include vitamin A, vitamin B1, vitamin B2, vitamin B6, vitamin B12, vitamin C, vitamin D, vitamin E, vitamin H, vitamin K, folic acid, the like, or combinations thereof.

The compositions and dosage forms described herein can include a variety of pharmaceutically acceptable carriers known in the art. At its most basic level, in some embodiments, a carrier can include water. In some embodiments, the water can be deoxygenated water. Non-limited examples of components that can be included as components of the pharmaceutical carrier include lipophilic surfactants, hydrophilic surfactants, triglycerides, fatty acid, or fatty acid glycerides, polymers, and combinations thereof.

In some embodiments, the carrier of the composition can include a lipophilic additive. In some embodiments, the lipophilic additive can comprise at least about 50 wt% of the pharmaceutically acceptable carrier. Non-limiting examples of lipophilic additives can include lipophilic surfactants, triglycerides, tocopherol, tocopherol derivatives and combinations thereof. In one embodiment, the lipophilic additive can include a fatty acid or fatty acid glyceride. In another embodiment, lipophilic additive can include the fatty acid glyceride, and the fatty acid glyceride can be a monoglyceride, a diglyceride, or mixtures thereof. Non-limiting examples of fatty acid glycerides that can be used in the pharmaceutical compositions and dosage forms of the present disclosure include monoglycerides and/or diglycerides derived from sources such as maize oil, poppy seed oil, safflower oil, sunflower oil, borage seed oil, peppermint oil, coconut oil, palm kernel oil, castor oil, and mixtures thereof. In one embodiment, the pharmaceutical composition or dosage form thereof comprises 50% by weight or less of a triglyceride. In a specific embodiment, the pharmaceutical composition or dosage form thereof, comprises less than 50% by weight of castor oil. In another embodiment, the composition includes 10 wt% or less of triglycerides. In a further embodiment, the composition includes 5 wt% or less of triglycerides. In a still a further embodiment, the composition includes about 3 wt% or less of triglycerides. In still a further embodiment, the composition includes about 1 wt% or less of triglycerides. In another embodiment, the composition is free or substantially free of triglycerides. In another embodiment, the composition and dosage forms are free of phytosterols and phytosterol fatty acid esters.

In another embodiment, the lipophilic additive can include a lipophilic surfactant. As used herein a surfactant is considered to be a lipophilic surfactant when it has an HLB value of 10 or less. Various lipophilic surfactants can be used including, but not limited to mono-, di- glycerides of fatty acids like glyceryl monolinoleate (e.g. Maisine® 35-1), mono- and di glycerides of caprylic, capric acid (e.g. CapmulⓇ MCM), glyceryl monooleate, reaction mixtures of alcohols or polyalcohols with a variety of natural and/or hydrogenated oils such as PEG-5 hydrogenated castor oil, PEG-7 hydrogenated castor oil, PEG-9 hydrogenated castor oil, PEG-6 corn oil (e.g. Labrafil® M 2125 CS), PEG-6 almond oil (e.g. Labrafil®M 1966 CS), PEG-6 apricot kernel oil (e.g. Labrafil®M 1944 CS), PEG-6 olive oil (e.g.Labrafil®M 1980 CS), PEG-6 peanut oil (e.g. Labrafil®M 1969 CS), PEG-6 hydrogenated palm kernel oil (e.g. Labrafil®. M 2130 BS), PEG-6 palm kernel oil (e.g. Labrafil® M 2130 CS), PEG-6 triolein (e.g. Labrafil® M 2735 CS), PEG-8 corn oil (e.g. Labrafil® WL 2609 BS), PEG-20 corn glycerides (e.g. Crovol® M40), PEG-20 almond glycerides (e.g. Crovol® A40), lipophilic polyoxyethylene-polyoxypropylene block co-polymers (e.g. Pluronic® L92, L101, L121 etc.); propylene glycol fatty acid esters, such as propylene glycol monolaurate (e.g. Lauroglycol FCC), propylene glycol ricinoleate (e.g. Propymuls), propylene glycol monooleate (e.g. Myverol P-06), propylene glycol dicaprylate/dicaprate (e.g. Captex® 200), and propylene glycol dioctanoate (e.g. Captex® 800), propylene glycol mono- caprylate (e.g. Capryol® 90); propylene glycol oleate (e.g. Lutrol OP2000); propylene glycol myristate; propylene glycol mono stearate; propylene glycol hydroxy stearate; propylene glycol ricinoleate ; propylene glycol isostearate; propylene glycol mono-oleate; propylene glycol dicaprylate/dicaprate; propylene glycol dioctanoate; propylene glycol caprylate-caprate; propylene glycol dilaurate; propylene glycol distearate; propylene glycol dicaprylate; propylene glycol dicaprate; mixtures of propylene glycol esters and glycerol esters such as mixtures composed of the oleic acid esters of propylene glycol and glycerol (e.g. Arlacel® 186); sterol and sterol derivatives such as cholesterol, sitosterol, phytosterol, phytosterol fatty acid esters, PEG-5 soya sterol, PEG-10 soya sterol, PEG-20 soya sterol, and the like; glyceryl palmitostearate, glyceryl stearate, glyceryl distearate, glyceryl monostearate, or a combination thereof; sorbitan fatty acid esters such as sorbitan monolaurate (e.g. Arlacel 20), sorbitan monopalmitate (e.g. Span-40), sorbitan monooleate (e.g. Span-80), sorbitan monostearate, and sorbitan tristearate, sorbitan monolaurate, sorbitan monopalmitate, sorbitan monooleate, sorbitan trioleate, sorbitan sesquioleate, sorbitan tristearate, sorbitan monoisostearate, sorbitan sesquistearate, and the like; fatty acids such as capric acid, caprylic acid, oleic acid, linoleic acid, myristic acid, menthol, menthol derivatives, lecithin, phosphatidyl choline, bile salts, and the like, and mixtures thereof. It can be noted that some lipophilic surfactants may also function as a solubilizer component of the compositions and dosage forms.

In one embodiment, the lipophilic surfactant can be selected from the group consisting of glyceryl monolinoleate (e.g. Maisine® 35-1), mono- and di glycerides of caprylic, capric acid (e.g. Capmul® MCM), glyceryl monooleate, propylene glycol mono caprylate, propylene glycol oleate, propylene glycol monostearate, propylene glycol monolaurate, propylene glycol mono-oleate, propylene glycol dicaprylate/dicaprate, sorbitan monooleate, PEG-5 hydrogenated castor oil, PEG-7 hydrogenated castor oil, PEG-9 hydrogenated castor oil, PEG-6 corn oil, PEG-6 almond oil, PEG-6 apricot kernel oil, PEG-6 olive oil, PEG-6 peanut oil, PEG-6 hydrogenated palm kernel oil, sorbitan monolaurate (e.g. Arlacel 20), sorbitan monopalmitate, sorbitan monooleate , sorbitan monostearate, sorbitan tristearate, sorbitan monolaurate, sorbitan monopalmitate, sorbitan monooleate, sorbitan trioleate, sorbitan sesquioleate, sorbitan tristearate, sorbitan monoisostearate, and combinations thereof. In some embodiments, the lipophilic surfactants can comprise at least about 50 wt% of the total pharmaceutically acceptable carrier. It should be noted that the combinations of two or more lipophilic surfactants from the same or different classes therein are also within the scope of this disclosure and are together can be referred to as the lipophilic surfactant, unless otherwise stated.

In embodiments of the present disclosure, the pharmaceutical compositions or dosage forms can include a hydrophilic additive. In one embodiment, hydrophilic additive is a selected from the group consisting of hydrophilic surfactant, celluloses - such as hydroxypropyl celluloses low molecular weight, low viscosity types (e.g. Methocel® E5, E6, E10 E15, LV100 etc. grades) and hydroxypropyl celluloses having higher molecular weight, medium to high viscosity (e.g. Methocel® K4M, K15M, K100M etc); polyvinylpyrrolidones (e.g. Kollidon k17, K30 etc); polyvinyl acetates and combinations thereof.

In one embodiment, the hydrophilic additive can be a hydrophilic surfactant. A surfactant is considered to be a hydrophilic surfactant when it has an HLB value of greater than 10. Non-limiting examples of hydrophilic surfactants include non-ionic surfactants, ionic surfactants and zwitterionic surfactants. Specifically, the hydrophilic surfactants include, but are not limited to, alcohol-oil transesterification products; polyoxyethylene hydrogenated vegetable oils; polyoxyethylene vegetable oils; alkyl sulphate salts, dioctyl sulfosuccinate salts; polyethylene glycol fatty acids esters; polyethylene glycol fatty acids mono- and di- ester mixtures; polysorbates, polyethylene glycol derivatives of tocopherol and the like. It should be noted that the combinations of two or more hydrophilic surfactants from the same or different classes are within the scope of this disclosure and are together can be referred to as the hydrophilic surfactant unless explicitly specified. In one embodiment, the hydrophilic additive can be a hydrophilic surfactant. Non-limiting examples of hydrophilic surfactants can include PEG-8 caprylic/capric glycerides, lauroyl macrogol-32 glyceride, stearoyl macrogol glyceride, PEG-40 hydrogenated castor oil, PEG-35 castor oil, sodium lauryl sulfate, sodium dioctyl sulfosuccinate, polyethylene glycol fatty acids mono- and di- ester mixtures, polysorbate 80, polysorbate 20, polyethylene glycol 1000 tocopherol succinate, phytosterols, phytosterol fatty acid esters, and mixtures thereof.

In some embodiments, surfactants utilized in the pharmaceutical compositions described herein include sterols and derivatives of sterols. In various embodiments, these surfactants are hydrophilic or lipophilic. Examples of hydrophilic sterol surfactants are lanosterol PEG-24 cholesterol ether (e.g. Solulan C-24, Amerchol), PEG-30 soya sterol (e.g. Nikkol BPS-30, from Nikko), PEG-25 phyto sterol (e.g. Nikkol BPSH-25 from Nikko), PEG-30 cholestanol (e.g. Nikkol DHC, from Nikko). Examples of Lipophilic Sterol Surfactants are Cholesterol, sitosterol, Phytosterol (e.g. GENEROL series from Henkel), PEG-5 soya sterol (e.g. Nikkol BPS-S, from Nikko), PEG-10 soya sterol (e.g. Nikkol BPS-10 from Nikko), PEG-20 soya sterol (e.g. Nikkol BPS-20 from Nikko).

In one embodiment, the hydrophilic surfactant can comprise at least about 20% of the total pharmaceutical carrier. In another embodiment, the hydrophilic surfactant can comprise at least about 5 wt% of the carrier. In another embodiment, the hydrophilic surfactant can comprise less than 5 wt% of the carrier.

In another embodiment, the compositions or the dosage forms of the current disclosure includes SOD, wherein the SOD comprises about 0.0001 wt% to about 50 wt% of the composition or the dosage form, and wherein the composition includes about 50 wt% to about 100 wt% of lipophilic additive and 0 wt% to about 50 wt% of hydrophilic additive. In a specific embodiment, the lipophilic additive can be lipophilic surfactant and the hydrophilic additive can be hydrophilic surfactant. In one embodiment, the hydrophilic surfactant can comprise at least about 20% of the composition. In another embodiment, the hydrophilic surfactant can comprise at least about 5 wt% of the composition. In another embodiment, the hydrophilic surfactant can comprise less than 5 wt% of the composition.

In some embodiments, the pharmaceutical compositions or the dosage form can include both a lipophilic surfactant and hydrophilic surfactant. In one embodiment, the lipophilic surfactant and hydrophilic surfactant can be present in amounts such that the ratio of amount (wt%) of lipophilic surfactant to amount (wt%) of hydrophilic surfactant is greater than 2:1. In still another embodiment, the lipophilic surfactant and hydrophilic surfactant can be present in amounts such that the ratio of amount (wt%) of lipophilic surfactant to amount (wt%) of hydrophilic surfactant is at least 6.5:1.

In some examples, the support agent can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In some examples, the support agent can can include an emollient. Non-limiting examples of emollients can include, but are not limited to, aloe vera, lanolin, urea, petrolatum, shea butter, cocoa butter, mineral oil, paraffin, beeswax, squalene, jojoba oil, coconut oil, sesame oil, almond oil, cetyl alcohol, stearyl alcohol, olive oil, oleic acid, triethylhexanoin, glycerol, sorbitol, propylene glycol, cyclomethicone, dimethicone, the like, or combinations thereof.

In some examples, the support agent can include an adjuvant. Non-limiting examples of adjuvants can include, but are not limited to, analgesic adjuvants, aluminum hydroxide, aluminum phosphate, calcium phosphate hydroxide, paraffin oil, Bordetella pertussis, mycobacterium bovis, toxoids, squalene, detergents, plant saponins, cytokines, and combinations thereof.

### Manufacturing and Stabilization

In yet another embodiment, a method of manufacturing a superoxide dismutase composition can comprise combining a superoxide dismutase (SOD) powder with deoxygenated water to form an SOD solution. In another aspect, the method can further comprise minimizing exposure of the SOD solution to reactive oxygen species (ROS). In one example, the exposure of the SOD solution to ROS can be minimized by minimizing an amount of ROS in a headspace of a container. In another aspect, the method can include combining the superoxide dismutase solution with a stabilizing agent, as discussed herein. In one aspect, the SOD can be one or more of human recombinant SOD (rhSOD), SOD1, SOD2, SOD3, or a combination thereof.

In yet another embodiment, a superoxide dismutase delivery system can include a container configured to minimize an amount of reactive oxygen species (ROS) in the container, and a combination of superoxide dismutase (SOD) and a stabilizing carrier within the container. In one aspect, the container can be configured to minimize the amount of ROS in a headspace of the container. In another aspect, the amount of ROS in the container can be less than about 5% of the container volume. The container can include an inert compound including one or more of: nitrogen gas, helium gas, neon gas, argon gas, krypton gas, xenon gas, radon gas, a non-reactive compound, a non-oxidizing compound, the like, or combinations thereof. In another aspect, the container can be a bag-on-valve container, a bag-in-bag container, a piston can container, or a can-in-can container.

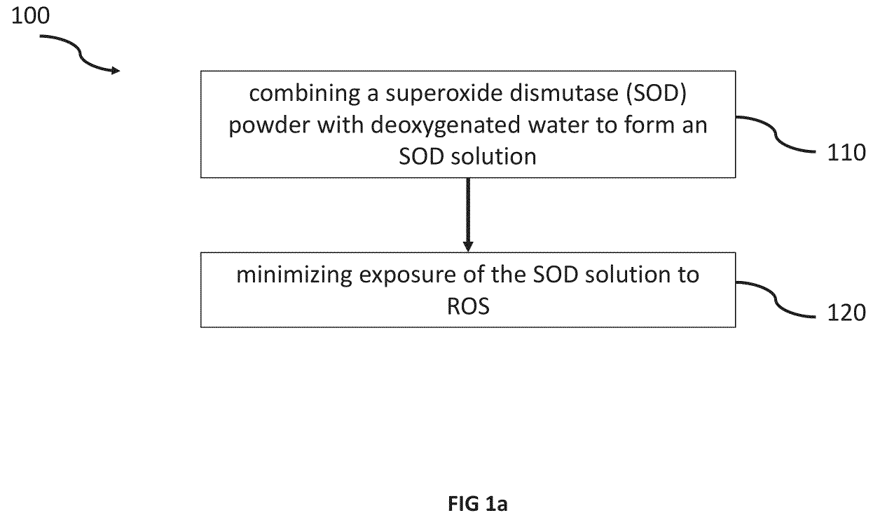

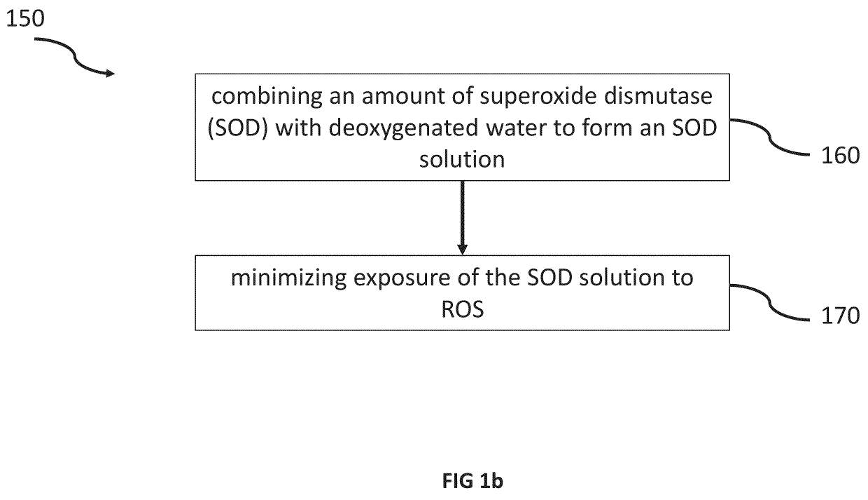

In some embodiments, as depicted in the flowchart in FIG. 1a, a method of manufacturing 100 a superoxide dismutase composition is provided. In one example, the method can comprise combining a superoxide dismutase (SOD) powder with deoxygenated water to form an SOD solution, as shown in block 110. In another example, the method can comprise minimizing exposure of the SOD solution to ROS, as shown in block 120. In a further aspect, minimizing an amount of ROS in a headspace of a container containing the SOD solution can minimize exposure of the SOD solution to ROS.

In a further embodiment, the method can include combining the superoxide dismutase solution with a stabilizing agent selected from the group consisting essentially of: phospholipids, glycolipids, cholesterol, triglycerides, fatty acids, fatty acid glycerides, surfactants, or a combination thereof.

In one example, deoxygenated water can be formed by removing dissolved O₂ gases from a water solvent. In one example, the water solvent can include a 20 mM phosphate buffer. In one example, ROS exposure of the SOD solution can be minimized by continuously deoxygenating water by an inert gas bubbling, with the deoxygenated water and SOD solution temperature maintained to avoid any re-oxygenation.

In one example, dissolved O₂ can be removed from water by one or more of: boiling water or reduced or atmospheric pressure, purging water with an inert gas, sonication of water under reduced pressure, mechanical means such as agitation and filtration, or gas removal based on differential pressure created through a duct, lumen, a sieve, or through a membrane, and the like. In another example, dissolved O₂ can be removed from water by decompression, and the residual oxygen can be removed by nitrogen.

In one example, the minimizing the exposure of the SOD to ROS can include minimizing an amount of oxygen in a container containing the SOD solution. The amount of ROS in a container containing the SOD solution can be minimized in various ways. The container can be filled at room temperature (e.g., in the range of about 15° C. to about 30° C.). The operation can be performed at atmospheric pressure, i.e., without employing any substantial vacuum before, during, or after the filling process. In some aspects, the filling at atmospheric pressure can include an overlay or blanketing of the container of use with an inert gas such as nitrogen or argon before or after the SOD solution has been introduced into the container. The container can be stoppered and sealed.

In one example, the container as used herein can be a container that is made of stainless steel or other suitable non-reactive material. Such containers may include glass-lined containers, polymeric bags, or other suitable containers that are generally employed in the compounding of pharmaceutical liquid preparations. In another example, the container can be a glass or plastic vial which contains the composition. Depending on the sterility restrictions, these vials may be further sterilized using heat, steam, gas, or radiation, among others.

In yet a further embodiment, a superoxide dismutase delivery system can include a container configured to minimize an amount of reactive oxygen species (ROS) in the container, and a combination of superoxide dismutase (SOD) and a stabilizing carrier within the container. In one aspect, the container can be further configured to minimize the amount of ROS in a headspace of the container. In one example, the amount of ROS in the container is less than about 5% of the container volume. In another example, the container can include an inert compound including one or more of: nitrogen gas, helium gas, neon gas, argon gas, krypton gas, xenon gas, radon gas, a non-reactive compound, a non-oxidizing compound, the like, or combinations thereof. The container can be a bag-on-valve container, a bag-in-bag container, a piston can container, or a can-in-can container.

In an example, the operations of production of a bag-on-valve, or can-in-can can comprise: (1) insert the bag on valve assembly, or the internal can, into the external can; (2) crimp the valve onto the external can; (3) fill the bag, or internal can, with the SOD solution; (4) add a propellant into the space between the can and the bag, or internal can (during crimping or following crimping); (5) assemble an actuator suitable for dispensing the SOD solution. In an example, the operations for production of a bag-on-valve container, a bag-in-bag container, a piston can container, or a can-in-can container can vary based on their different mechanical structures, but the principles of assembling the system, filling it with the SOD solution, and closing it can have similarities to the operations described in the preceding.

In another embodiment, as depicted in the flowchart in FIG. 1b, a method 150 of stabilizing a superoxide dismutase (SOD) can include combining an amount of SOD with deoxygenated water to form an SOD solution as in block 160, and minimizing exposure of the SOD solution to reactive oxygen species (ROS), as in block 170. In one aspect, the deoxygenated water can have less than one or more of: about 10 ppm of oxygen, about 5 ppm of oxygen, about 3 ppm of oxygen, about 1 ppm of oxygen, or combinations thereof.

In another aspect, the SOD can be present in the SOD solution at a concentration of from about 0.0001 wt% to about 10 wt%. In one example, the SOD can be present in the SOD solution at a concentration of from about 0.0001 wt% to about 1 wt%. In another example, the SOD can be present in the SOD solution at a concentration of from about 0.0001 wt% to about 0.5 wt%. In another example, the SOD can be present in the SOD solution at a concentration of from about 0.0001 wt% to about 0.1 wt%. In one more example, the SOD can be present in the SOD solution at a concentration of from about 0.001 wt% to about 0.1 wt%.

In one aspect, when the SOD is present in the SOD solution at a concentration of 0.02 wt%: (a) more than about 80% of the SOD can remain in a reactive form after 2 weeks when stored at room temperature and 50-75% relative humidity; or (b) more than about 80% of the SOD can remain in a reactive form after 4 weeks when stored at room temperature and 75% relative humidity, or (c) more than about 80% of the SOD can remain in a reactive form after 2 weeks when stored at 40° C. and 75% relative humidity, or (d) more than about 80% of the SOD can remain in a reactive form after 4 weeks when stored at 40° C. and 75% relative humidity.

In another aspect, when the SOD is present in the SOD solution at a concentration of 0.02 wt%: (a) more than about 90% of the SOD can remain in a reactive form after 2 weeks when stored at room temperature and 50-75% relative humidity, or (b) more than about 90% of the SOD can remain in a reactive form after 4 weeks when stored at room temperature and 50-75% relative humidity, or (c) more than about 90% of the SOD can remain in a reactive form after 2 weeks when stored at 40° C. and 75% relative humidity, or (d) more than about 90% of the SOD can remain in a reactive form after 4 weeks when stored at 40° C. and 75% relative humidity.

In one aspect, more than about 50% of the SOD can remain in a reactive form after 365 days when stored at room temperature and 50-75% relative humidity. In another aspect, more than about 80% of the SOD can remain in a reactive form after 365 days when stored at room temperature and 75% relative humidity. In one more aspect, more than about 90% of the SOD can remain in a reactive form after 365 days when stored at room temperature and 75% relative humidity.

In another aspect, the SOD solution can further comprise lidocaine. In one aspect, when the SOD is present in the SOD solution at a concentration of 0.02 wt% and the lidocaine is present in the SOD solution at a concentration of 0.5 wt%: (a) more than about 80% of the SOD can remain in a reactive form after 2 weeks when stored at room temperature and 50-75% relative humidity, or (b) more than about 80% of the SOD can remain in a reactive form after 4 weeks when stored at room temperature and 50-75% relative humidity, or (c) more than about 80% of the SOD can remain in a reactive form after 2 weeks when stored at 40° C. and 75% relative humidity, or (d) more than about 80% of the SOD can remain in a reactive form after 4 weeks when stored at 40° C. and 75% relative humidity.

In one aspect, when the SOD is present in the SOD solution at a concentration of 0.02 wt% and the lidocaine is present in the SOD solution at a concentration of 0.5 wt%: (a) more than about 90% of the SOD can remain in a reactive form after 2 weeks when stored at room temperature and 50-75% relative humidity, or (b) more than about 90% of the SOD can remain in a reactive form after 4 weeks when stored at room temperature and 75% relative humidity, or (c) more than about 90% of the SOD can remain in a reactive form after 2 weeks when stored at 40° C. and 75% relative humidity, or (d) more than about 90% of the SOD can remain in a reactive form after 4 weeks when stored at 40° C. and 75% relative humidity.

In one aspect, the SOD solution can be substantially free of ethanol, capsaicin, menthol, carbomer, the like, and combinations thereof.

### Formulations and Dosage Forms

The compositions and the dosage forms of the current disclosure can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof. The dosage forms of the current disclosure can comprise a transdermal dosage form, a parenteral dosage form, a topical dosage form, an oral dosage form, a nebulizer dosage form, a transmucosal dosage form, and combinations thereof.

Molecules with a molecular mass of greater than about 500 Daltons are not expected to pass through the epidermis. SOD1 has a molecular mass of about 17.3-23 kilodaltons (kDa). Unexpectedly, topical application of a composition of the current disclosure can have a rapid localized therapeutic effect on a subject.

In addition, it is unexpected that a relatively small dose of SOD (e.g., one or two drops of 0.02 wt% SOD solution) can have such a rapid localized therapeutic effect on a subject. This is unexpected for a few reasons: (a) a molecule with the size of SOD (e.g., greater than 17 kDa) is at least 50 times the size molecules that can pass through the epidermis to have a rapid localized therapeutic effect, (b) the SOD molecule is subject to degradation by oxidation (e.g., unstable on route to a treatment situs), (c) the amount of SOD administered is unexpectedly low, (d) the therapeutic effect on a subject is rapid (e.g., 30-60 seconds in many cases), and (e) the therapeutic effect arises from a topical SOD composition that is substantially free of a skin penetration enhancer.

In one embodiment, a topical dosage form for administration of superoxide dismutase (SOD) can include an amount of SOD and a stabilizing carrier which is suitable for topical administration. In one aspect, the carrier can be one or more of a gel, a cream, an ointment, a lotion, a liniment, or a paste. In another aspect, the SOD can be present in an amount of about 15 mcg to about 1500 mcg per 1 mL to 5 mL. In a further aspect, the topical dosage form can be controlled release controlled by a pH-sensitive activator. The topical dosage forms can include a carrier comprising one or more of a solution, a gel, a cream, an ointment, a liniment, a paste, or a lotion.

In one example, the topical dosage form can comprise a combination of a therapeutically effective amount of superoxide dismutase (SOD) with a stabilizing carrier that is suitable for topical administration. The topical dosage form can include the properties of the topical composition as disclosed herein.

In a further embodiment, a transdermal dosage form for administration of superoxide dismutase (SOD) can include an amount of SOD and a stabilizing carrier which is suitable for transdermal administration. The carrier can include one or more of water, a polymer having surfactant properties, a polymer having thickening properties, or a solvent. In a further aspect, the transdermal dosage form can include a penetration enhancer. Further, the SOD can be present in an amount of about 15 mcg to about 1500 mcg per 1 mL to 5 mL. The transdermal dosage form can be controlled release controlled by a pH sensitive activator.

In one embodiment, a pH sensitive activator can be a pH sensitive liposome. In one example, pH-sensitive liposomes can be prepared by adding pH-sensitive units to the liposome dispersion or by mixing pH-sensitive lipids and polymers during the preparation of vesicles.

In a further embodiment, a polymorphic lipid can be used to prepare pH-sensitive liposomes, including, but not limited to: unsaturated phosphatidylethanolamine (PE), such as diacetylenic-phosphatidyl-ethanolamine (DAPE), palmitoyl-oleoyl-phosphatidyl-ethanolamine (POPE) and dioleoyl-phosphatidyl-ethanolamine (DOPE). In one aspect, DOPE can be combined with amphiphiles that act as stabilizers at a selected pH, such as oleic acid (OA), cholesteryl hemisuccinate (CHEMS), and palmitoyl homocysteine (PHC), to formulate pH-sensitive liposomes.

In one example, the pH sensitive liposome can control release at a pH from about 5 and 9. In another example, the pH sensitive liposome can control release at a pH from about 6 and 8. In one specific example, the pH sensitive liposome can be configured to delay release at a pH of from about 5 to about 6 (e.g., as found on human skin), and activate release at a pH of from about 6 to about 8 (e.g., as found in tissue below the epidermis).

The transdermal dosage form can further comprise a penetration enhancer. Non-limiting examples of a penetration enhancer can include, but are not limited to, ethanol, propylene glycol, oleic acid and other fatty acids, azone, terpenes, terpenoids, bile acids, isopropyl myristate and other fatty esters, dimethyl sulphoxides, N-methyl-2-pyrrolidone and other pyrrolidones, the like, or combinations thereof.

The transdermal dosage forms can include a carrier including one or more of water, a polymer having surfactant properties, a polymer having thickening properties, or a solvent. The transdermal dosage form can further comprise a penetration enhancer. The penetration enhancer can be present in an amount of about less than 5% by weight of composition. In another example, the penetration enhancer can be present in an amount of about less than 4% by weight of composition. In another example, the penetration enhancer can be present in an amount of about less than 3% by weight of composition. In another example, the penetration enhancer can be present in an amount of about less than 2% by weight of composition. In another example, the penetration enhancer can be present in an amount of about less than 1% by weight of composition.

In some examples, the transdermal dosage form can include a polymer having surfactant or emulsifying properties. Non-limiting examples of a polymer having surfactant or emulsifying properties can include, but are not limited to, hydrophobically modified polyacrylic acid commercially under the tradename PemulenTM TR-I and TR-2 by Lubrizol Corp., water-soluble or water-swellable copolymers based on acrylamidoalkyl sulfonic acid and cyclic N-vinylcarboxamides commercially available under the tradename Aristoflex® AVC by Clariant Corporation; water-soluble or water-swellable copolymers based on acrylamidoalkyl sulfonic acid and hydrophobically modified methacrylic acid commercially available under the tradename Aristoflex® HMB by Clariant Corporation and a homopolymer of acrylamidoalkyl sulfonic acid commercially available under the tradename Granthix APP by Grant Industries, Inc.

Other materials that may be suitable polymeric surfactants can include ethylene oxide/ propylene oxide block copolymers, sold under the trade name PLURONIC®, available from BASF Corporation of Parsippany, NJ., modified cellulose polymers such as those modified cellulose polymers described by the trade name KLUCEL®, available from Hercules Corporation of Wilmington, DE. Some examples include hydrophobically modified polyacrylic acid, acrylamidoalkyl sulfonic acid, cyclic N-vinylcarboxamides, acrylamidoalkyl sulfonic acid, hydrophobically modified methacrylic acid, a homopolymer of acrylamidoalkyl sulfonic acid, or combinations thereof as polymeric emulsifiers; and monomeric anionic surfactants, monomeric amphoteric surfactants, or combinations thereof as foaming agents. Some more examples are compositions that include hydrophobically modified polyacrylic acid; water-soluble or water-swellable copolymers based on acrylamidoalkyl sulfonic acid, cyclic N-vinylcarboxamides; water-soluble or water-swellable copolymers based on acrylamidoalkyl sulfonic acid, hydrophobically modified methacrylic acid; a homopolymer of acrylamidoalkyl sulfonic acid, or combinations thereof as polymeric emulsifiers, and include a betaine as the foaming surfactant. Some examples are compositions that include copolymers based on acrylamidoalkylsulfonic acids and cyclic N-vinylcarboxamides and/or linear N- vinylcarboxamides (e.g., Aristoflex® AVC and Aristoflex® HMB from Clariant Corporation) as polymeric emulsifiers and a betaine as foaming surfactant.

In some examples, the transdermal dosage form can include a polymer having thickening properties. Non-limiting examples of a polymer having thickening properties can include, but are not limited to, a hydrophobically modified cross-linked acrylate copolymer (Carbopol® Ultrez 20). Other polymers having similar properties may also be used. Non-limiting examples of polymers having thickening properties can include PEG-150 distearate, PEG-7 glyceryl cocoate, PEG-200 hydrogenated glyceryl palmitate, PEG-120 methyl glucose dioleate, carboxymethylene polymer, carboxyvinyl polymer, acrylates, C₁₀-C₃₀ alkyl acrylate crosspolymers, and combinations thereof.

In a further embodiment, a transmucosal dosage form for administration of superoxide dismutase (SOD) can include an amount of SOD and a stabilizing carrier which is suitable for transmucosal administration. In another aspect, the SOD can be present in an amount of about 15 mcg to about 1500 mcg per 1 mL to 5 mL. The dosage form can further include one or more of an adhesive layer, a bio-erodible matrix, or a solution. The dosage form can be suitable for nasal, vaginal, or rectal administration.

In some examples, the transmucosal dosage form can include an adhesive, such as acrylic adhesives, polyisobutylene adhesives, silicon adhesives, hydrogel adhesives, the like, or combinations thereof.

In some examples, the transmucosal dosage form can include a bio-erodable matrix including, but are not limited to, biodegradable constituents, such as polylactic acid, poly(lactic-co-glycolic) acid, polyglycolic acid, poly(caprolactone), hyaluronic acid, polyhydroxybutyrate, polyvinyl alcohol, polyvinylpyrrolidone, carbomers, polyacrylic acid, polyoxyethylene/polyoxypropylene copolymers, other copolymers, albumins, casein, zein, collagen, other proteins, glucose, sucrose, maltose, trehalose, amylose, dextrose, fructose, mannose, galactose, other sugars, erythritol, threitol, arabitol, xylitol, ribitol, mannitol, sorbitol, galactitol, fucitol, iditol, inositol, volemitol, isomalt, maltitol, lactitol, maltotriitol, maltotetraitol, polyglycitol, other sugar alcohols, chondroitin and/or other glycosaminoglycans, inulin, starches, acacia gum, agar, carboxymethyl cellulose, methyl cellulose, ethyl cellulose, alginates, carrageenan, cassia gums, cellulose gums, chitin, chitosan, curdlan, gelatin, dextran, fibrin, fulcelleran, gellan gum, ghatti gum, guar gum, tragacanth, karaya gum, locust bean gum, pectin, starch, tara gum, xanthan gum, and other polysaccharides, and functionalized derivatives of any of the above, copolymers thereof, the like, or mixtures thereof.

Non-limiting examples of solubilizers and/or emulsifiers can include water, ethanol, propylene glycol, ethylene glycol, glycerin, polyethylene glycol, banzalkonium chloride, benzethonium chloride, cetylpyridinium chloride, docusate sodium, nonoxynol-9, octoxynol, polyoxyethylene polyoxypropylene co-polymers, polyoxyl castor oils, polyoxyl hydrogenated castor oils, polyoxyl oleyl ethers, polyoxyl cetylstearyl ethers, polyoxyl stearates, polysorbates, sodium lauryl sulfate, sorbitan monolaurate, sorbitan monooleate, sorbitan monopalmitate, sorbitan monostearate, tyloxapol, the like, or combinations thereof.

In some examples, the solubilizer can also include a hydrocarbon or fatty substance, such as petrolatum, microcrystalline wax, paraffin wax, mineral oil, ceresi, coconut oil, bees wax, olive oil, lanolin, peanut oil, spermaceti wax, sesame oil, almond oil, hydrogenated castor oils, cotton seed oil, soybean oil, corn oil, hydrogenated sulfated castor oils, cetyl alcohol, stearyl alcohol, oleyl alcohol, lauryl alcohol, myristyl alcohol, stearic acid, oleic acid, palmitic acid, lauraic acid, ethyl oleate, isopropyl myristicate, the like, or combinations thereof.

In some examples, the solubilizer can include a silicon, such as polydimethylsiloxanes, methicones, dimethylpropylsiloxanes, methyl phenyl polysiloxanes, steryl esters of dimethyl polysiloxanes, ethoxylated dimethicones, ethoxylated methicones, the like, or combinations thereof.

In some additional examples, the therapeutic composition can include a dispersant and/or thickening agent, such as polyacrylic acids (e.g., Carbopols, for example), gelatin, pectin, tragacanth, methyl cellulose, hydroxylethylcellulose, hydroxypropylcellulose, HPMC, CMC, alginate, starch, polyvinyl alcohol, polyvinyl pyrrolidone, co-polymers of polyoxyethylene and polyoxypropylene, polyethylene glycol, the like, or combinations thereof.

The pH adjusters, tonicity agents, and preservatives in the topical, transdermal, or transmucosal therapeutic composition can generally include those pH adjusters and buffering agents, tonicity agents, and preservative agents listed above, or any other suitable pH adjusters, buffering agent, tonicity agent, or preservative for a particular formulation and/or use thereof. In some examples, the therapeutic composition can also include fumed silica, mica, talc, titanium dioxide, kaolin, aluminum glycinate, ethylenediaminetetraacetic acid, fragrances, colorants, other components as described above, the like, or combinations thereof.

The oral compositions of the present disclosure can be formulated to take any dosage form commonly known in the pharmaceutical arts such as solution, emulsion, granules, tablet or capsule. In one embodiment, the oral dosage form can be a capsule having a pharmaceutical composition of the present disclosure disposed therein. Both soft and hard gelatin and non-gelatin capsules can be used. The capsule size can be any size known in the art and can vary depending on the desired dosage amount. For instance, in one embodiment, the capsule can be a hard gelatin capsule having a fill volume of about 0.25 mL to about 1.1 mL. Similarly, in another embodiment, the capsule can be a soft gelatin capsule having a fill volume of about 0.25 mL to about 1.5 mL.

In a specific embodiment, the compositions of the current disclosure can be formulated in the form of granules, powder mixtures or tablets. In a specific embodiment, the SOD present in the dosage form can be present in the form of nanoparticles or amorphous particles, or a mixture of both. In another specific embodiment, the SOD present in these dosage form can be present in the form of crystalline, non-crystalline or amorphous particles or a mixture thereof having an average particle size of about 2000 nm or less, 1500 nm or less, 1000 nm, 800 nm or less, 600 nm or less, 500 nm or less, 400 nm or less, 300 nm or less, 250 nm or less, 200 nm or less, 100 nm or less, 50 nm or less, or 25 nm or less.

In another aspect of the disclosure, the oral pharmaceutical compositions and/or capsule dosage forms, namely the capsule fill, can include a solidifying agent. A solidifying agent is a pharmaceutically acceptable additive that is in a solid physical state at 20° C. Typically, solidifying agents facilitate the solidification of the pharmaceutical compositions of the present disclosure at temperatures around room temperature. The compositions and capsule fill of the present disclosure, including those with solidifying agents, can be non-liquid at standard temperature and pressure. In one embodiment, the composition and capsule fill can be semi-solid at standard temperature and pressure. In yet another embodiment, the composition and capsule fill can be solid at standard temperature and pressure. When present, the solidifying agent can comprise from about 0.1 wt% to about 25 wt% of the pharmaceutical composition or capsule dosage form. In another embodiment, the solidifying agent can comprise about 2 wt% to about 20 wt% of the composition or capsule dosage form. In yet a further embodiment, the solidifying agent can comprise about 3 wt% to about 15 wt% of the composition or capsule dosage form. In still a further embodiment, the solidifying agent can comprise about 3 wt% to about 9 wt% of the capsule fill. In yet a further embodiment, the solidifying agent can comprise 6 wt% to 9 wt% of the capsule fill. In one embodiment, the solidifying agent can melt at a temperature of about 45° C. to about 75° C. Non-limiting examples of solidifying agents that can be used include polyethylene glycols; sorbitol; gelatin; stearic acid; cetyl alcohol; cetosterayl alcohol; paraffin wax; polyvinyl alcohol; glyceryl stearates; glyceryl distearate; glyceryl monostearate; glyceryl palmitostearate; glyceryl behenate; waxes; hydrogenated castor oil; hydrogenated vegetable oil; bees wax, microcrystalline wax; sterols; phytosterols; phytosterols fatty acid esters, cholesterol and mixtures thereof. In one embodiment, the solidifying agent includes a polyethylene glycol (PEG) having molecular weight from about 1000 to about 20,000 and their mixtures. In another embodiment the solidifying agent includes one or more selected from the group consisting of polyethylene glycol; gelatin; stearic acid; polyvinyl alcohol; glyceryl stearates; glyceryl distearate; glyceryl monostearate; glyceryl palmitostearate; hydrogenated castor oil; hydrogenated vegetable oil and cholesterol.

In another specific embodiment, a solution of the SOD can be in a carrier (e.g. lipophilic additive or hydrophilic additive or combinations thereof). Such solutions can be dispersed (e.g. by adsorption) in a solid carrier such colloidal silicon dioxide, lactose, calcium silicate, magnesium aluminum silicates, microcrystalline cellulose or combinations thereof, etc., and prepared as powder mixtures or granules or pellets to be disposed/filled into capsules or sachets or admixed with tableting aids and compressed as tablets. Such sachets, capsules or tablets can also be formulated to contain an additional amount of the SOD in crystalline and/or non-crystalline form, such that in the final composition or dosage form the total SOD amount exists as a combination of at least two of the forms including solution, crystalline and non-crystalline forms, at about 20° C. or at about human body temperature or at 30° C. or above 30° C. including the range 30° C. to 40° C. In a further embodiment, these dosage forms provide serum SOD levels and the pharmacokinetic parameters disclosed in the current disclosure for the SOD upon single administration or two consecutive administrations or upon steady state.

In a further embodiment, the oral pharmaceutical composition can be formulated as dosage form to be administered to provide a daily SOD dose of about 15 mcg to about 1500 mcg per 1 mL to 5 mL based on single unit or multiple unit dosing. In a specific embodiment, a single unit dosing comprises administering the entire required dose of the SOD per administration time in the form of one unit dosage form; whereby the subject has to consume one unit dosage from per administration. In another specific embodiment, a multiple unit dosing comprises administering the entire required dose of the SOD per administration time in the form of two or more unit dosage form; whereby the subject has to consume two, three, four or more unit dosages, per administration.

The dosage forms can be immediate release or controlled release (e.g., extended release, targeted release, pulsatile release, delayed release, etc.) or combinations thereof. When formulated as oral dosage forms, including the disclosed solution, capsule, or tablet dosage forms, the dosage forms can be formulated for once-a-day administration or for twice-a-day administration. The compositions and oral dosage forms can also be formulated for administration with a meal, including once-a-day administration with a meal. While the compositions dosage forms disclosed herein can be administered with a meal, a meal is not necessarily required.

In one embodiment, an oral dosage form for administration of superoxide dismutase (SOD) can comprise an amount of superoxide dismutase (SOD) and a stabilizing carrier which is suitable for oral administration. In another embodiment, the oral dosage form can include SOD present in an amount of about 15 mcg to about 1500 mcg per 1 mL to 5 mL. The stabilizing carrier can be present in an amount from about 1 mL to about 5 mL.

In one aspect, the carrier can comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In one embodiment, a parenteral dosage form for administration of a stable superoxide dismutase (SOD) can include an amount of SOD and a stabilizing carrier which is suitable for parenteral administration. In one aspect, the carrier can include one or more of water, a tonicity agent, a buffering agent, a preservative, or a combination thereof. In one aspect, the SOD is present in an amount of about 15 mcg to about 1500 mcg per 1 mL to 5 mL. The dosage form can be suitable for administration via intradermal injection, subcutaneous injection, intramuscular injection, intraperitoneal injection, or intravenous injection.

In some examples, the parenteral dosage form can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco’s PBS, Alsever’s solution, Tris-buffered saline (TBS), water, balanced salt solutions (BSS), such as Hank’s BSS, Earle’s BSS, Grey’s BSS, Puck’s BSS, Simm’s BSS, Tyrode’s BSS, and BSS Plus, the like, or combinations thereof. The tonicity agent can be used to provide an appropriate tonicity of the composition. In one aspect, the tonicity of the composition can be from about 250 to about 350 milliosmoles/liter (mOsm/L). In another aspect, the tonicity of the composition can be from about 277 to about 310 mOsm/L.

In some examples, the parenteral dosage form can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8. In another example, the pH of the therapeutic composition can be from about 5 to about 6.

In some additional examples, the pharmaceutically acceptable carrier can be formulated for administration via inhalation. In some examples, such formulations can include a propellant, such as hydrofluoralkanes, such as HFA134a, HFA227, or other suitable propellant. In yet other examples, the composition can be formulated for administration via nebulization. In either case, the composition can also include a variety of solubilizing agents, such as those described above. In other examples, the composition can be formulated as a dry powder aerosol. In some examples, the composition can include a particulate carrier and/or other particulate excipients, such as lactose, mannitol, other crystalline sugars, fumed silica, magnesium stearate, amino acids, the like, or combinations thereof.

In some specific examples, the pharmaceutically acceptable carrier can be formulated to provide a composition for ocular administration. Non-limiting examples can include topical application to the eye in the form of a drop, a gel, a film, an insert, a sponge, an ointment, the like, or a combination thereof. In yet other examples, the composition can be formulated for intraocular injection or implantation in the form of a solution, a depot, a scaffold, the like, or a combination thereof. Ocular compositions can include a variety of excipients, such as water, a tonicity agent, a thickening agent, a biodegradable polymer, a solubilizing agent, an emulsifier, a preservative, the like, or other suitable component, or a combination thereof.

It is noted that a number of compositional excipients are disclosed above with specific reference to particular types of formulations. However, it is noted that any excipients disclosed herein, or other suitable excipients, can be used with any type of therapeutic composition, where suitable, whether or not a particular excipient or type of excipient is specifically described in connection with that type of therapeutic composition. Therefore, the compositions described herein can be formulated in a variety of ways for various modes of administration.

In one embodiment, the composition or dosage form (e.g., capsule or tablet) can be administered with a meal, such as a meal that provides about 200 to about 1000 calories of energy of which 20-35% come from fats in the meal. In another embodiment, the composition or the dosage form can be administered with a standard meal. In another embodiment, the composition or capsule dosage form can be administered with a meal that provides about 50% of the calories derived from the fat. In another embodiment, the composition or the dosage form can be administered with a high-fat, high calorie meal. In another embodiment, the composition or the dosage form can be administered with a meal that provides about 500 to about 1000 calories of energy. In another embodiment, the composition or the dosage form can be administered with a meal that provides about 400 to about 700 calories derived from the fat therein. The compositional make-up of the meals that are administered can vary depending on the tastes and dietary needs of a subject. However, in some situations it may be beneficial to administer the compositions and dosage forms with meals that provide no fat or up to about 50 g of fat. In one embodiment, the meal can provide about 10 g to about 50 g of fat. In yet a further embodiment, the meal can provide about 20-35 g of fat.

In another embodiment, the composition or the dosage form can be administered orally with a meal that provides of the current disclosure can be administered to a subject, along with a meal such as breakfast, snack, food, lunch, dinner etc. In a specific embodiment, the meal can comprise about 15-55% fat. In another specific embodiment, the meal can comprise about 20-35% fat. In another specific embodiment, the meal can comprise about 20-55% fat.

In another embodiment, the oral pharmaceutical composition or dosage form can be formulated such that upon continuous once-a-day administration to each subject in a group of at least 25 subjects for a period of at least 28 days, 50% or less of the subjects in the group have: (a) a steady state serum SOD1 concentration that falls below 700 ,ug/mL for more than 7 hours per day; (b) a steady state serum SOD2 concentration that falls below 75 ng/mL for more than 7 hours per day; (c) a steady state serum SOD3 concentration that falls below 20 ng/mL for more than 7 hours per day.

In another embodiment, the oral pharmaceutical composition or dosage form can be formulated such that upon continuous once-a-day administration to each subject in a group of at least 25 subjects for a period of at least 28 days, 25% or less of the subjects in the group have a steady state serum SOD1 concentration that falls below 500 ,ug/mL for more than 7 hours per day; ; (b) a steady state serum SOD2 concentration that falls below 50 ng/mL for more than 7 hours per day; (c) a steady state serum SOD3 concentration that falls below 15 ng/mL for more than 7 hours per day.

In another embodiment, the oral pharmaceutical composition or dosage form can be formulated such that upon continuous twice daily administration to each subject in a group of at least 25 subjects for a period of at least 28 days, less than 50% of subjects in the group have: (a) a steady state serum SOD1 concentration that falls below 700 µg/mL for more than 3.5 hours per day; (b) a steady state serum SOD2 concentration that falls below 75 ng/mL for more than 3.5 hours per day; (c) a steady state serum SOD3 concentration that falls below 20 ng/mL for more than 3.5 hours per day.

In another embodiment, the pharmaceutical composition or dosage form can be formulated such that upon continuous twice daily administration to each subject in a group of at least 25 subjects for a period of at least 28 days, 25% or less of subjects in the group have: (a) a steady state serum SOD1 concentration that falls below 500 µg/mL for more than 3.5 hours per day; (b) a steady state serum SOD2 concentration that falls below 50 ng/mL for more than 3.5 hours per day; (c) a steady state serum SOD3 concentration that falls below 15 ng/mL for more than 3.5 hours per day.

As discussed above, the present disclosure also provides for a method of treating a human subject in need of SOD therapy is provided. The method can include administering any of the pharmaceutical compositions or dosage forms disclosed herein. The pharmaceutical compositions and the dosage forms of the present disclosure can be used to treat any condition associated with SOD deficiency. Examples of conditions associated with SOD deficiency that can be treated using the dosage forms and/or compositions of the present disclosure include, but are not limited to: cardiovascular disease, hypertension, atherosclerosis, myocardial infarction, chronic heart failure (CHF), lung disease, acute respiratory distress syndrome, chronic obstructive pulmonary disease, asthma, acute kidney injury, chronic kidney disease, kidney transplantation recovery, acute liver injury, diabetic retinopathy, cataract, inflammation, toxin-induced poisoning, cancer, radiation poisoning, arthritis, rheumatoid arthritis, osteoarthritis, interstitial cystitis, gout, paraquat-induced poisoning, Parkinson’s disease, Alzheimer’s disease, dengue fever, cancer, Down’s syndrome, and combinations thereof.

In some embodiments, the compositions and dosage forms of the SOD of the current disclosure improves at least one of biological absorption and metabolic stability of the SOD. In one embodiment, the biological absorption of the SOD is intestinal lymphatic absorption.

In another embodiment, the compositions or dosage of the current disclosure having SOD can be administered for maintaining or controlling physiological levels of SOD in a subject in need of SOD therapy. In a further embodiment, the compositions or dosage of the current disclosure having SOD can be administered in combination with other active agents for maintaining or controlling physiological levels of other active agents in a subject in need of SOD therapy. In a further embodiment, the compositions or dosage of the current disclosure can have at least one of the immediate release, modified release, and targeted delivery properties in various regions of the GI tract and can be administered for maintaining or controlling physiological levels of SOD in a subject in need of SOD therapy.

In some examples, the SOD can be co-administered with menthol or capsaicin. In this example, the menthol or capsaicin can be co-administered with the SOD by dispensing the SOD from a first chamber of a dispenser and dispensing the menthol or capsaicin from a second chamber of a dispenser without mixing the SOD with the menthol or capsaicin until a dispensing event. By keeping the SOD separate from the menthol or capsaicin until the dispensing event, the degradative effect that menthol or capsaicin can have upon mixing with SOD can be ameliorated.

Subjects that can be treated by the SOD compositions and dosage form of the present disclosure can be any human subject in need thereof. In one embodiment, the human subject is an adult of at least age 20. In a further embodiment, the human subject can be at least age 50. In yet a further embodiment, the human subject can be an adult of at least age 65.

### Methods

In yet another embodiment, a method of treating a condition in a subject that is responsive to treatment with superoxide dismutase can comprise administering a therapeutically effective amount of the composition disclosed herein to the subject. In one aspect, the condition can be amyotrophic lateral sclerosis (ALS). In one aspect, the condition can be senescence. In another aspect, the condition can be a member selected from a group consisting of: cardiovascular disease, inflammation, toxin-induced poisoning, cancer, lung disease, radiation poisoning, the like, and combinations thereof. In yet another aspect, the condition can be a member selected from a group consisting of: arthritis, rheumatoid arthritis, osteoarthritis, acute respiratory distress syndrome, atherosclerosis, interstitial cystitis, gout, paraquat-induced poisoning, kidney transplantation, heart attack, the like, and combinations thereof.

In another example, the condition can be one or more of: ALS, SOD genetic errors, osteoarthritis, interstitial cystitis, fibromyalgia, pain (e.g., back, leg, ankle, joint, wound, chronic, or the like), neuropathy (e.g., chemo-induced peripheral neuropathy (CIPN), diabetic, arthritic, osteoarthritic, or the like), shingles, post-herpetic neuralgia, trigeminal neuralgia, ankylosing spondylitis, edema, eczema, inflammation, erectile dysfunction, the like, or a combination thereof.

In one example, when the condition is one or more of: ALS, SOD genetic errors, interstitial cystitis, fibromyalgia, ankylosing spondylitis, or inflammation, then the dosage form can have from about 0.1 ml to about 2.5 ml of the composition. In another example, the dosage form can have from about 0.5 ml to about 1 ml of the composition. In another example, a daily dose can be from about 0.1 ml to about 10 ml of the composition. In another example, a daily dose can be from about 0.5 ml to about 4 ml of the composition.

In another example, when the condition is one or more of: osteoarthritis, localized pain, neuropathy, shingles, postherpetic neuralgia, trigeminal neuralgia, edema, eczema, or erectile dysfunction, then the dosage form can have from about 25 ul to about 1 ml of the composition. In another example, the dosage form can have from about 100 ul ml to about 0.75 ml of the composition. In another example, a daily dose can be from about 25 ul ml to about 5 ml of the composition. In another example, a daily dose can be from about 100 ul to about 1 ml of the composition.

In one more example, the dosage interval can vary based on the number of previous doses administered in a selected time window and a duration of administration. In one example, the therapeutically effective amount of the topical composition can be administered to a subject at least two different dosage intervals. The first dosage interval (e.g., the time between the first dose administered and the second dose administered in a day) can be a selected interval between 30 minute and 12 hours and the second dosage interval (e.g., the time between the second dose administered and the third dose administered in a day) can be a selected interval between 30 minutes and 12 hours. In some examples, the first dosage interval can be lower than a second dosage interval. For example, a subject who receives a first dose at 9 am, a second dose at 12pm, and a third dose at 10pm would have a first dosage interval of about 3 hours and a second dosage interval of about 10 hours. In this example, the dosage interval can lengthen over the course of a day. In other example, the dosage interval can lengthen over the course of a week, 2 weeks, 4 weeks, 8 weeks, 3 months, 6 months, 1 years, the like, and combinations thereof.

The dosage interval can increase throughout the course of a day from 3 hours for a first dosage interval to 10 hours for a second dosage interval because the administration of the topical composition can interfere with the pain pathway of a subject. In a first administration, the composition can interfere with the pain pathway by interfering with transient receptor potential (TRP) cation channels. In a second administration, the composition can further interfere with the pain pathway (e.g., the TRP cation channels). Before the third administration occurs (e.g., at a longer dosage interval between the second and third administration than the dosage interval between the first and second administration), then the pain pathway has been adequately inhibited to provide pain relief for a longer duration.

### ALS

Amyotrophic lateral sclerosis (ALS) is a neurodegenerative disease that can result in progressive loss of motor neurons that control voluntary muscles. Some early symptoms include muscle stiffness, muscle twitches, muscles weakness, and muscle waste. About 90% of ALS cases have no known cause and treatments have been limited.

In one embodiment, a method of treating ALS can comprise administering a therapeutically effective amount of a topical composition to a subject.

In one aspect, the topical composition can be a dosage form having from about 0.25 ml to about 1 ml of 0.02 wt% SOD. In one example, the dosage form can have from about 1.5 ml to about 2.5 ml of 0.02 wt% SOD. In another example, the dosage form can have from about 2.5 ml to about 4 ml of 0.02 wt% SOD. In one more example, the dosage form can have from about 4 ml to about 6 ml of 0.02 wt% SOD.

In another aspect, a daily dose of the topical composition can be from about .25 ml to about 3 ml of 0.02 wt% SOD. In one example, the daily dose of the topical composition can be from about 0.5 ml to about 2 ml of 0.02 wt% SOD. In another example, the daily dose of the topical composition can be from about 0.25 ml to about 4 ml of 0.02 wt% SOD.

In one aspect, the topical composition can be a dosage form having from about 0.25 ml to about 1 ml of 0.04% wt% SOD. In one example, the dosage form can have from about 1.5 ml to about 2.5 ml of 0.04 wt% SOD. In another example, the dosage form can have from about 3 ml to about 4 ml of 0.04 wt% SOD. In one more example, the dosage form can have from about 4 ml to about 6 ml of 0.04 wt% SOD.

In another aspect, a daily dose of the topical composition can be from about .25 ml to about 3 ml of 0.04 wt% SOD. In one example, the daily dose of the topical composition can be from about 0.5 ml to about 2 ml of 0.04 wt% SOD. In another example, the daily dose of the topical composition can be from about 0.25 ml to about 4 ml of 0.04 wt% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with ALS (e.g., muscle stiffness, muscle twitches, muscles weakness, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating ALS can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### SOD Genetic Errors

SOD genetic errors can be the cause of various types of diseases including amyotrophic lateral sclerosis (ALS), neural disorders seen in Down syndrome, thalassemia, Acute Respiratory Distress Syndrome, Chronic Obsructive Pulmonary Disease, and the like.

In one embodiment, a method of treating SOD genetic errors can comprise administering a therapeutically effective amount of a topical composition to a subject.

In one aspect, the topical composition can be a dosage form having from about 0.25 ml to about 1 ml of 0.02 wt% SOD. In one example, the dosage form can have from about 1.5 ml to about 2.5 ml of 0.02 wt% SOD. In another example, the dosage form can have from about 2.5 ml to about 4 ml of 0.02 wt% SOD. In one more example, the dosage form can have from about 4 ml to about 6 ml of 0.02 wt% SOD.

In another aspect, a daily dose of the topical composition can be from about .25 ml to about 3 ml of 0.02 wt% SOD. In one example, the daily dose of the topical composition can be from about 0.5 ml to about 2 ml of 0.02 wt% SOD. In another example, the daily dose of the topical composition can be from about 0.25 ml to about 4 ml of 0.02 wt% SOD.

In one aspect, the topical composition can be a dosage form having from about 0.25 ml to about 1 ml of 0.04% wt% SOD. In one example, the dosage form can have from about 1.5 ml to about 2.5 ml of 0.04 wt% SOD. In another example, the dosage form can have from about 3 ml to about 4 ml of 0.04 wt% SOD. In one more example, the dosage form can have from about 4 ml to about 6 ml of 0.04 wt% SOD.

In another aspect, a daily dose of the topical composition can be from about .25 ml to about 3 ml of 0.04 wt% SOD. In one example, the daily dose of the topical composition can be from about 0.5 ml to about 2 ml of 0.04 wt% SOD. In another example, the daily dose of the topical composition can be from about 0.25 ml to about 4 ml of 0.04 wt% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with SOD genetic errors. In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating SOD genetic errors can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Osteoarthritis

Arthritis affects millions of people worldwide. One form of arthritis –osteoarthritis – can occur when the protective cartilage that cushions the ends of the bones wears down over time. The disorder can affect joints in hands, knees, hips, and the spine, among others. Some symptoms are pain, stiffness, tenderness, loss of mobility, grating sensation, bone spurs, swelling, the like, and combinations thereof.

In one embodiment, a method of treating osteoarthritis can comprise administering a therapeutically effective amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with osteoarthritis (e.g., pain, stiffness, tenderness, loss of flexibility, grating sensation, bone spurs, swelling, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating osteoarthritis can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof. In some embodiments, the analgesic can be a histamine or a salt thereof, such as histamine dihydrochloride. In some embodiments, the histamine (e.g. histamine dihydrochloride can be present in the composition in an amount of from about O.lmg/g - 5 mg/g. In other embodiments, the amount can be about 0.25 mg/g (0.025% of the formulation).

### Interstitial Cystitis

Many causes may be responsible for causing interstitial cystitis such as a defect in the protective lining of the bladder, autoimmune reactions, heredity, infection, or allergy. The pain caused by interstitial cystitis can range from mild discomfort to severe pain. Some of the symptoms associated with interstitial cystitis include pain in the pelvis, a persistent urge to urinate, frequent urination, and the like.

In one embodiment, a method of treating interstitial cystitis can comprise administering a therapeutically effective amount of a topical composition to a subject.

In one aspect, the topical composition can be a dosage form having from about 0.25 ml to about 1 ml of 0.02 wt% SOD. In one example, the dosage form can have from about 1.5 ml to about 2.5 ml of 0.02 wt% SOD. In another example, the dosage form can have from about 2.5 ml to about 4 ml of 0.02 wt% SOD. In one more example, the dosage form can have from about 4 ml to about 6 ml of 0.02 wt% SOD.

In another aspect, a daily dose of the topical composition can be from about .25 ml to about 3 ml of 0.02 wt% SOD. In one example, the daily dose of the topical composition can be from about 0.5 ml to about 2 ml of 0.02 wt% SOD. In another example, the daily dose of the topical composition can be from about 0.25 ml to about 4 ml of 0.02 wt% SOD.

In one aspect, the topical composition can be a dosage form having from about 0.25 ml to about 1 ml of 0.04% wt% SOD. In one example, the dosage form can have from about 1.5 ml to about 2.5 ml of 0.04 wt% SOD. In another example, the dosage form can have from about 3 ml to about 4 ml of 0.04 wt% SOD. In one more example, the dosage form can have from about 4 ml to about 6 ml of 0.04 wt% SOD.

In another aspect, a daily dose of the topical composition can be from about .25 ml to about 3 ml of 0.04 wt% SOD. In one example, the daily dose of the topical composition can be from about 0.5 ml to about 2 ml of 0.04 wt% SOD. In another example, the daily dose of the topical composition can be from about 0.25 ml to about 4 ml of 0.04 wt% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with interstitial cystitis (e.g., pain in the pelvis, a persistent urge to urinate, frequent urination, pain or discomfort when the bladder filles or empties, pain during sexual intercourse, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating interstitial cystitis can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Fibromyalgia

Fibromyalgia is a condition that affects pain perception processing and affects about 2% of the adult population. Some symptoms associated with fibromyalgia include widespread pain, sleep disorders, fatigue, emotional distress, and mental distress. Some other symptoms can include tingling, numbness, pain in the face, digestive issues, and the like.

In one embodiment, a method of treating fibromyalgia can comprise administering a therapeutically effective amount of a topical composition to a subject.

In one aspect, the topical composition can be a dosage form having from about 0.25 ml to about 1 ml of 0.02 wt% SOD. In one example, the dosage form can have from about 1.5 ml to about 2.5 ml of 0.02 wt% SOD. In another example, the dosage form can have from about 2.5 ml to about 4 ml of 0.02 wt% SOD. In one more example, the dosage form can have from about 4 ml to about 6 ml of 0.02 wt% SOD.

In another aspect, a daily dose of the topical composition can be from about .25 ml to about 3 ml of 0.02 wt% SOD. In one example, the daily dose of the topical composition can be from about 0.5 ml to about 2 ml of 0.02 wt% SOD. In another example, the daily dose of the topical composition can be from about 0.25 ml to about 4 ml of 0.02 wt% SOD.

In one aspect, the topical composition can be a dosage form having from about 0.25 ml to about 1 ml of 0.04% wt% SOD. In one example, the dosage form can have from about 1.5 ml to about 2.5 ml of 0.04 wt% SOD. In another example, the dosage form can have from about 3 ml to about 4 ml of 0.04 wt% SOD. In one more example, the dosage form can have from about 4 ml to about 6 ml of 0.04 wt% SOD.

In another aspect, a daily dose of the topical composition can be from about .25 ml to about 3 ml of 0.04 wt% SOD. In one example, the daily dose of the topical composition can be from about 0.5 ml to about 2 ml of 0.04 wt% SOD. In another example, the daily dose of the topical composition can be from about 0.25 ml to about 4 ml of 0.04 wt% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with fibromyalgia (e.g., pain, stiffness, fatigue, depressions, anxiety, sleep disorders, attention deficits, headaches, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating fibromyalgia can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Pain

Pain can be a symptom of an underlying condition that can be characterized as chronic or acute with underlying mechanisms involving (a) nociceptors, (b) the somatosensory system, (c) nociplastic pain, (d) or psychogenic pain. In many cases, acute pain is managed with medications such as analgesics. Some treatments involve opioids – a class of drug that include heroin, fentanyl, oxycodone, hydrocodone, codeine, morphine, and others. However, misuse of opioids can lead to dependence, addiction, overdose, and death.

In one embodiment, a method of treating pain can comprise administering a therapeutically effective amount of a topical composition to a subject. In one example, the pain can be one or more of chronic pain, back pain, leg pain, ankle pain, joint pain, arthritic pain, wound pain, the like, or combinations thereof. In another example, the pain can be acute pain.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with pain. In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating pain can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Neuropathy

Damage or dysfunction in one or more nerves that results in numbness, tingling, muscle weakness, or muscle pain is a characteristic of peripheral neuropathy. There are various types of neuropathy (e.g., mononeuropathy, multifocal neuropathy, or polyneuropathy) and various causes (e.g., diabetes, trauma, autoimmune disorders, infections, medication, poisons, vascular disorders, abnormal vitamin levels, genetics, of the like). Some symptoms include tingling, sharp pain, burning pain, throbbing pain, stabbing pain, electric-like pain, falling, loss of coordination, lack of feeling, muscle weakness, or the like.

In one embodiment, a method of treating neuropathy can comprise administering a therapeutically effective amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with neuropathy (e.g., include tingling, sharp pain, burning pain, throbbing pain, stabbing pain, electric-like pain, falling, loss of coordination, lack of feeling, muscle weakness, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating neuropathy can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Shingles

The reactivation of the varicella zoster virus (VZV) can result in the virus traveling from the nerve body to nerve endings in skin which can produce blisters. Some early symptoms of shingles include headache, fever, and malaise, which can be followed by burning pain, itching, oversensitive skin, tingling, pricking, numbness, stinging, throbbing, stabbing, or the like. In the skin, the virus can cause local inflammation and blistering.

In one embodiment, a method of treating shingles can comprise administering a therapeutically effective amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with shingles (e.g., burning pain, itching, oversensitive skin, tingling, pricking, numbness, stinging, throbbing, stabbing, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating shingles can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Post-Herpetic Neuralgia

In many cases, a subject with shingles may overcome the disease over the course of a month without any complications thereafter. But for some people pain can be felt long after the blisters arising from shingles have healed. This can occur when the nerve fibers are inflamed or damaged during the shingles outbreak. Some of the symptoms associated with post-herpetic neuralgia can include pain felt when touched, long-lasting pain, burning, itching, tingling, aching, shooting feelings, numbness, or headaches.

In one embodiment, a method of treating post-herpetic neuralgia can comprise administering a therapeutically effective amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with post-herpetic neuralgia (e.g., stabbing pain, burning, itching, tingling, aching, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating post-herpetic neuralgia can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Trigeminal Neuralgia

Trigeminal neuralgia is a chronic pain condition that can affect the trigeminal nerve which carries sensation signals from the face to the brain. In some cases, a person with trigeminal neuralgia can have excruciating pain with even the slightest activity involving the face (e.g., brushing teeth or shaving).

In one embodiment, a method of treating trigeminal neuralgia can comprise administering a therapeutically effectives amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with trigeminal neuralgia (e.g., episodes of stabbing pain on the face). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating trigeminal neuralgia can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Ankylosing Spondylitis

Ankylosing spondylitis is a type of arthritis that can cause pain and stiffness in the spine. Over time, the disease can cause small bones in the spine to fuse together which can result in a less flexible spine with a hunched-forward posture. When the ribs are affected, breathing can be difficult.

In one embodiment, a method of treating ankylosing spondylitis can comprise administering a therapeutically effective amount of a topical composition to a subject.

In one aspect, the topical composition can be a dosage form having from about 0.25 ml to about 1 ml of 0.02 wt% SOD. In one example, the dosage form can have from about 1.5 ml to about 2.5 ml of 0.02 wt% SOD. In another example, the dosage form can have from about 2.5 ml to about 4 ml of 0.02 wt% SOD. In one more example, the dosage form can have from about 4 ml to about 6 ml of 0.02 wt% SOD.

In another aspect, a daily dose of the topical composition can be from about .25 ml to about 3 ml of 0.02 wt% SOD. In one example, the daily dose of the topical composition can be from about 0.5 ml to about 2 ml of 0.02 wt% SOD. In another example, the daily dose of the topical composition can be from about 0.25 ml to about 4 ml of 0.02 wt% SOD.

In one aspect, the topical composition can be a dosage form having from about 0.25 ml to about 1 ml of 0.04% wt% SOD. In one example, the dosage form can have from about 1.5 ml to about 2.5 ml of 0.04 wt% SOD. In another example, the dosage form can have from about 3 ml to about 4 ml of 0.04 wt% SOD. In one more example, the dosage form can have from about 4 ml to about 6 ml of 0.04 wt% SOD.

In another aspect, a daily dose of the topical composition can be from about .25 ml to about 3 ml of 0.04 wt% SOD. In one example, the daily dose of the topical composition can be from about 0.5 ml to about 2 ml of 0.04 wt% SOD. In another example, the daily dose of the topical composition can be from about 0.25 ml to about 4 ml of 0.04 wt% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with ankylosing spondylitis (e.g., pain and stiffness proximal to the spine, lower back, hips, pelvis, breastbone, ribs, shoulder joints, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating ankylosing spondylitis can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Edema

Edema is swelling that can be caused by excess fluid trapped in a body’s tissues. Some signs of edema include swelling or puffiness of the tissue under the skin, stretched skin, skins that retains simples after pressing, or an increase in abdominal size.

In one embodiment, a method of treating edema can comprise administering a therapeutically effective amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with edema (e.g., surface area or volume associated with swelling, stretched skin, dimples after pressing skin, increase in abdominal size, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating edema can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Eczema

Eczema occurs when patches of skin become rough or inflamed which can include blisters, itching, bleeding, irritation. Eczema can be caused by soaps, detergents, wool, skin infections, dry skin, low humidity, heat, sweating, emotional stress, allergens, or the like.

In one embodiment, a method of treating eczema can comprise administering a therapeutically effective amount of a topical composition to a subject.

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with eczema (e.g., surface area or volume associated with dry skin, itching, redness, raised bumps, cracked skin, swollen skin, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating eczema can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Inflammation

The regulation of neutrophil apoptosis plays a role in the resolution of inflammation. In some cases, the activated neutrophils can be removed via apoptosis. SOD may induce neutrophil apoptosis and hydrogen peroxide (which can be produced by activated SOD from ROS) can also mediate ROS-induced neutrophil apoptosis. As such, inflammation and related disorders can be responsive to treatment with SOD.

In one embodiment, a method of treating inflammation can comprise administering a therapeutically effective amount of a topical composition to a subject.

In one aspect, the topical composition can be a dosage form having from about 0.25 ml to about 1 ml of 0.02 wt% SOD. In one example, the dosage form can have from about 1.5 ml to about 2.5 ml of 0.02 wt% SOD. In another example, the dosage form can have from about 2.5 ml to about 4 ml of 0.02 wt% SOD. In one more example, the dosage form can have from about 4 ml to about 6 ml of 0.02 wt% SOD.

In another aspect, a daily dose of the topical composition can be from about .25 ml to about 3 ml of 0.02 wt% SOD. In one example, the daily dose of the topical composition can be from about 0.5 ml to about 2 ml of 0.02 wt% SOD. In another example, the daily dose of the topical composition can be from about 0.25 ml to about 4 ml of 0.02 wt% SOD.

In one aspect, the topical composition can be a dosage form having from about 0.25 ml to about 1 ml of 0.04% wt% SOD. In one example, the dosage form can have from about 1.5 ml to about 2.5 ml of 0.04 wt% SOD. In another example, the dosage form can have from about 3 ml to about 4 ml of 0.04 wt% SOD. In one more example, the dosage form can have from about 4 ml to about 6 ml of 0.04 wt% SOD.

In another aspect, a daily dose of the topical composition can be from about .25 ml to about 3 ml of 0.04 wt% SOD. In one example, the daily dose of the topical composition can be from about 0.5 ml to about 2 ml of 0.04 wt% SOD. In another example, the daily dose of the topical composition can be from about 0.25 ml to about 4 ml of 0.04 wt% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with inflammation (e.g., redness, swelling, pain, function, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating inflammation can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### Erectile Dysfunction

Erectile dysfunction is a debilitating disease that can affect adult males. In about 80% of cases, a physical cause can be identified. Some of these causes can include one or more of cardiovascular disease, diabetes mellitus, neurological issues, drug side effects. Nitric oxide plays a role in treating this disease, but levels of nitric oxide can be affected by reactive oxygen species. SOD can treat erectile dysfunction by reducing oxidative stress and allowing nitric oxide to properly function.

In one embodiment, a method of treating erectile dysfunction can comprise administering a therapeutically effective amount of a topical composition of SOD to a subject

In one aspect, the topical composition can be a dosage form having from about 25 ul to about 1 ml of formulation containing 0.02% wt/wt SOD. In one example, the dosage form can have from about 0.1 ml to about 0.75 ml of formulation containing 0.02% SOD. In another example, the dosage form can have from about 0.75 ml to about 5 ml of formulation containing 0.02% SOD.

In another aspect, a daily dose of the topical composition can be from about 25 ul to about 5 ml of formulation containing 0.02% SOD. In one example, the daily dose of the topical composition can be from about 100 ul to about 1 ml of formulation containing 0.02% SOD. In another example, the daily dose of the topical composition can be from about 1 ml to about 5 ml of formulation containing 0.02% SOD.

In yet another aspect, the therapeutically effective amount of the topical composition can be administered to the subject 1 to 10 times per day. In one example, the topical composition can be administered to the subject 1 to 8 times per day. In another example, the topical composition can be administered to the subject 1 to 6 times per day. In yet another example, the topical composition can be administered to the subject 3 to 5 times per day.

In one more aspect, the therapeutically effective amount of the topical composition can be administered to the subject according to a dosage regimen. In one example, the topical composition can be administered at least once per day for a duration of from about a single day to about 12 months. In another example, the topical composition can be administered at least once per day for a duration of from about a single day to about 6 months. In one more example, the topical composition can be administered at least once per day for a duration of from about a single day to about 3 months. In yet another example, the topical composition can be administered at least once per day for a duration of from about a single day to about 1 month.

Administering the therapeutically effective amount of the topical composition can reduce the symptoms associated with inflammation (e.g., time to reach erection, frequency of erections, length of erections, reduced interest in sexual activity, or the like). In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In another aspect, the treatment can provide a reduction in symptoms of at least 10% since commencement of the treatment. In one example, the treatment can provide a reduction in symptoms of at least 20% since commencement of the treatment. In one more example, the treatment can provide a reduction in symptoms of at least 30% since commencement of the treatment. In yet another example, the treatment can provide a reduction in symptoms of at least 50% since commencement of the treatment. In another example, the treatment can provide a reduction in symptoms of at least 80% since commencement of the treatment. In another example, the treatment can provide a reduction in symptoms of at least 90% since commencement of the treatment.

In one example, the method of treating erectile dysfunction can further comprise co-administering an active agent to the subject. The co-administration can be concomitant administration. The active agent can be in the same or a different composition as the SOD. The active agent can be a member selected from the group consisting of: an antioxidant, an antibiotic, an antiviral, an antitoxin agent, hydroxytyrosol (HXT), reduced glutathione (GSH), catalase, an anti-infective agent, an anti-tumor agent, an anti-inflammatory agent, an analgesic, an anesthetic, an anti-rheumatic agent, a growth factor, a cytokine, an amino acid, a protein, a vaccine, a hormone, a vitamin, oleuropein, the like, or combinations thereof.

### EXAMPLES

In one example, a superoxide dismutase composition can comprise a combination of a therapeutically effective amount of superoxide dismutase (SOD) with a stabilizing carrier.

In another example, the stabilizing carrier can comprise water.

In another example, the water can comprise deoxygenated water.

In another example, the deoxygenated water can have less than about 5 ppm of oxygen.

In another example, the composition can further comprise a support agent selected from the group consisting of: a stabilizing agent, a carrier, a preservative, an emollient, an adjuvant, the like, and combinations thereof.

In another example, the support agent can be a stabilizing agent.

In another example, the stabilizing agent can include an oxygen scavenger, a lipid, a synthetic polymer, a natural polymer, a polyelectrolyte, a protein, an amino acid, a co-polymer, an emulsion, a gel, an inert gas, or a combination thereof.

In another example, the stabilizing agent can be an encapsulating agent that forms a nanoemulsion, a liposome, a nanogel, or a combination thereof.

In another example, the encapsulating agent can be selected from the group consisting essentially of: chitosan, cyclodextrin, dextran, starch, silicon, tragacanth, and combinations thereof.

In another example, the stabilizing agent can form a stabilizing complex with the SOD.

In another example, the stabilizing agent can be a lipid selected from the group consisting essentially of: phospholipids, glycolipids, cholesterol, triglycerides, fatty acids, fatty acid glycerides, surfactants, or a combination thereof.

In another example, the lipid can form a liposome, an inverted micelle, or a combination thereof.

In another example, the composition can further comprise an additional active agent.

In another example, the additional active agent can be a member selected from the group consisting of: catalase, an antioxidant, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, the like, and combinations thereof.

In another example, the composition can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof.

In another example, the composition can comprise a dosage form comprising a transdermal dosage form, a topical dosage form, an oral dosage form, a parenteral dosage form, a nebulizer dosage form, a transmucosal dosage form, and combinations thereof.

In another example, the SOD can be present in the composition at a concentration of from about 0.0001 wt% to about 10 wt%.

In another example, more than about 80% of the SOD can remain in a reactive form after a period of about 365 days.

In one example, a method of manufacturing a superoxide dismutase composition can comprise: combining a superoxide dismutase (SOD) powder with deoxygenated water to form an SOD solution; and minimizing exposure of the SOD solution to reactive oxygen species (ROS).

In another example, the exposure of the SOD solution to ROS can include minimizing an amount of ROS in a headspace of a container containing the SOD solution.

In another example, the method can further comprise combining the superoxide dismutase solution with a stabilizing agent.

In another example, the stabilizing agent can be a lipid selected from the group consisting essentially of: phospholipids, glycolipids, cholesterol, triglycerides, fatty acids, fatty acid glycerides, surfactants, or a combination thereof.

In one example, a superoxide dismutase delivery system can comprise: a container configured to minimize an amount of reactive oxygen species (ROS) in the container; and a combination of superoxide dismutase (SOD) and a stabilizing carrier within the container.

In another example, the container can be further configured to minimize the amount of ROS in a headspace of the container.

In another example, the amount of ROS in the container can be less than about 5% of the container volume.

In another example, the container can include an inert compound including one or more of: nitrogen gas, helium gas, neon gas, argon gas, krypton gas, xenon gas, radon gas, a non-reactive compound, a non-oxidizing compound, the like, or combinations thereof.

In another example, the container can be a bag-on-valve container, a bag-in-bag container, a piston can container, or a can-in-can container.

In one example, a method of treating a condition in a subject that is responsive to treatment with superoxide dismutase can comprise: administering a therapeutically effective amount of a composition to the subject, wherein the composition comprises a combination of a therapeutically effective amount of superoxide dismutase (SOD) with a stabilizing carrier.

In another example, the condition can be senescence.

In another example, the condition can be a member selected from a group consisting of: cardiovascular disease, inflammation, toxin-induced poisoning, cancer, lung disease, radiation poisoning, the like, and combinations thereof.

In another example, the condition can be a member selected from a group consisting of: arthritis, rheumatoid arthritis, osteoarthritis, acute respiratory distress syndrome, atherosclerosis, interstitial cystitis, gout, paraquat-induced poisoning, kidney transplantation, heart attack, the like, and combinations thereof.

In one example, a topical dosage form for administration of superoxide dismutase (SOD) can comprise an amount of SOD and a stabilizing carrier which is suitable for topical administration.

In another example, the carrier can be one or more of a gel, a cream, an ointment, a lotion, a liniment, or a paste.

In another example, the SOD can be present in an amount of about 15 mcg to about 1500 mcg.

In another example, the topical dosage form can be controlled release.

In another example, the release can be controlled by a pH-sensitive activator.

In one example, a transdermal dosage form for administration of a stable superoxide dismutase (SOD) can comprise an amount of SOD and a stabilizing carrier which is suitable for transdermal administration.

In another example, the carrier can include one or more of water, a polymer having surfactant properties, a polymer having thickening properties, or a solvent.

In another example, the transdermal dosage form can comprise a penetration enhancer.

In another example, the SOD can be present in an amount of about 15 mcg to about 1500 mcg.

In another example, the transdermal dosage form can be controlled release.

In another example, the release can be controlled by a pH-sensitive activator.

In one example, an oral dosage form for administration of superoxide dismutase (SOD) can comprise an amount of superoxide dismutase (SOD) and a stabilizing carrier which is suitable for oral administration.

In another example, the carrier can comprise one or more of: binders, buffers, compacting aids, diluents, disintegrants, flavors, colorants, taste-masking agents, pH modifiers, lubricants, glidants, thickening agent, opacifying agent, humectants, desiccants, effervescing agents, sweeteners, plasticizing agents, wetting agents, and combinations thereof.

In another example, the SOD can be present in an amount of about 15 mcg to about 1500 mcg.

In another example, the oral dosage form can be a capsule or tablet.

In another example, the oral dosage form can be controlled release.

In one example, a parenteral dosage form for administration of a stable superoxide dismutase (SOD) can comprise an amount of SOD and a stabilizing carrier which is suitable for parenteral administration.

In another example, the carrier can comprise one or more of water, a tonicity agent, a buffering agent, a preservative, or a combination thereof.

In another example, the SOD can be present in an amount of about 15 mcg to about 1500 mcg.

In another example, the dosage form can be suitable for administration via intradermal injection, subcutaneous injection, intramuscular injection, intraperitoneal injection, or intravenous injection.

In one example, a transmucosal dosage form for administration of a stable superoxide dismutase (SOD) can comprise an amount of SOD and a stabilizing carrier which is suitable for transmucosal administration.

In another example, the SOD can be present in an amount of about 15 mcg to about 1500 mcg.

In another example, the transmucosal dosage form can comprise one or more of an adhesive layer, a bio-erodible matrix, or a solution.

In another example, the dosage form can be suitable for nasal, vaginal, or rectal administration.

In another example, the stabilizing carrier can comprise deoxygenated water.

In another example, the stabilizing carrier can comprise deoxygenated water having less than about 5 ppm of oxygen.

In another example, the composition can further comprise a support agent selected from the group consisting of a stabilizing agent, a carrier, a preservative, an emollient, an adjuvant, the like, and combinations thereof.

In another example, the dosage form can further comprise a stabilizing agent including an oxygen scavenger, a lipid, a synthetic polymer, a natural polymer, a polyelectrolyte, a protein, an amino acid, a co-polymer, an emulsion, a gel, an inert gas, or a combination thereof.

In another example, the dosage form can comprise a stabilizing agent that is an encapsulating agent that forms a nanoemulsion, a liposome, a nanogel, or a combination thereof.

In another example, the dosage form can comprise a stabilizing agent that is an encapsulating agent selected from the group consisting essentially of: chitosan, cyclodextrin, dextran, starch, silicon, tragacanth, and combinations thereof.

In another example, the dosage form can comprise a stabilizing agent that forms a stabilizing complex with the SOD.

In another example, the dosage form can comprise a stabilizing agent that is a lipid selected from the group consisting essentially of phospholipids, glycolipids, cholesterol, triglycerides, fatty acids, fatty acid glycerides, surfactants, or a combination thereof.

In another example, the lipid can form a liposome, an inverted micelle, or a combination thereof.

In another example, the dosage form can comprise an additional active agent selected from the group consisting of: catalase, an antioxidant, an anti-infective agent, an antibiotic, an anti-tumor agent, an anti-inflammatory agent, an analgesic, an anti-rheumatic agent, a growth factor, a cytokine, an amino acid, a protein, a vaccine, a hormone, a vitamin, the like, and combinations thereof.

In another example, the dosage form can be formulated as one of a solution, a suspension, an emulsion, a gel, a hydrogel, a thermo-responsive gel, a cream, an ointment, a paste, an adhesive, an erodible matrix, a liquid reservoir, a patch, a powder, a compressed powder, or a combination thereof.

In another example, the dosage form can comprise a transdermal dosage form, a topical dosage form, an oral dosage form, a parenteral dosage form, a nebulizer dosage form, a transmucosal dosage form, and combinations thereof.

In another example, the SOD can be present in the composition at a concentration of from about 0.0001 wt% to about 10 wt%.

In another example, more than about 80% of the SOD remains in a reactive form after a period of about 365 days.

In another example, a topical composition can comprise: a combination of a therapeutically effective amount of superoxide dismutase (SOD) with a stabilizing carrier that is suitable for topical administration.

In another example, the stabilizing carrier can comprise deoxygenated water.

In another example, the deoxygenated water can have less than one or more of: about 10 ppm of oxygen, about 5 ppm of oxygen, about 3 ppm of oxygen, about 1 ppm of oxygen, or combinations thereof.

In another example, the topical composition can further comprise a support agent selected from the group consisting of: a stabilizing agent, a preservative, an emollient, an adjuvant, and combinations thereof.

In another example, the support agent can be a stabilizing agent.

In another example, the stabilizing agent can include an oxygen scavenger, a lipid, a synthetic polymer, a natural polymer, a polyelectrolyte, a protein, an amino acid, a co-polymer, an emulsion, a gel, an inert gas, or a combination thereof.

In another example, the stabilizing agent can be an encapsulating agent that forms a nanoemulsion, a liposome, a nanogel, or a combination thereof.

In another example, the encapsulating agent can be selected from the group consisting essentially of: chitosan, cyclodextrin, dextran, starch, silicon, tragacanth, and combinations thereof.

In another example, the stabilizing agent can form a stabilizing complex with the SOD.

In another example, the stabilizing agent can be a lipid selected from the group consisting essentially of: phospholipids, glycolipids, cholesterol, triglycerides, fatty acids, fatty acid glycerides, surfactants, or a combination thereof.

In another example, the lipid can form a liposome, an inverted micelle, or a combination thereof.

In another example, the topical composition can further comprise an additional active agent.

In another example, the additional active agent can be a member selected from the group consisting of: a catalase, an antioxidant, an anti-infective agent, an antibiotic, an anti-tumor agent, an anti-inflammatory agent, an anesthetic, an analgesic, an anti-rheumatic agent, a growth factor, a cytokine, an amino acid, a protein, a vaccine, a hormone, a vitamin, and combinations thereof.

In another example, the additional active agent can be an anesthetic.

In another example, the anesthetic can be a member selected from the group consisting of: articaine, bupivacaine, cinchocaine, etidocaine, levobupivacaine, lidocaine, mepivacaine, prilocaine, ropivacaine, trimecaine, and combinations thereof.

In another example, the anesthetic can be lidocaine.

In another example, the additional active agent can be in the same or a different composition as the SOD.

In another example, the additional active agent can be present at a concentration of from about 0.0001 wt% to about 10 wt%.

In another example, the topical composition can have a pH of from about 6.8 to about 7.4.

In another example, the topical composition can have a viscosity of from about 1 cP to about 1000 cP.

In another example, the topical composition can be formulated as one of: a solution, a suspension, an emulsion, a gel, a hydrogel, a thermo-responsive gel, a cream, an ointment, a paste, an adhesive, a liquid reservoir, a patch, or a combination thereof.

In another example, the SOD can be present in the topical composition at a concentration of from about 0.0001 wt% to about 10 wt%.

In another example, when the SOD is present in the topical composition at a concentration of 0.02 wt%: more than about 80% of the SOD can remain in a reactive form after a period of about 2 weeks when stored at room temperature and 75% relative humidity; or more than about 80% of the SOD can remain in a reactive form after a period of about 2 weeks when stored at 40° C. and 75% relative humidity; or more than about 80% of the SOD can remain in a reactive form after a period of about 4 weeks when stored at room temperature and 75% relative humidity; or more than about 80% of the SOD can remain in a reactive form after a period of about 4 weeks when stored at 40° C. and 75% relative humidity.

In another example, the topical composition can consist essentially of SOD and deoxygenated water.

In another example, the topical composition can be substantially free of: ethanol, capsaicin, menthol, carbomer, and combinations thereof.

In another example, the SOD can comprise human recombinant SOD (rhSOD), SOD-1, SOD-2, SOD-3, or a combination thereof.

In another example, a method of treating a condition in a subject that is responsive to treatment with superoxide dismutase (SOD) can comprise: administering a therapeutically effective amount of the topical composition to the subject.

In another example, the condition can be pain.

In another example, the pain can be one or more of chronic pain, back pain, leg pain, ankle pain, joint pain, arthritic pain, wound pain, or combinations thereof.

In another example, the condition can be edema.

In another example, the condition can be inflammation.

In another example, the condition can be erectile dysfunction.

In another example, the condition can be interstitial cystitis.

In another example, the treatment can provide a reduction in symptoms of at least 10% within a selected amount of time after administration.

In another example, the topical composition can comprise a dosage form having from about 25 ul to about 10 ml of SOD.

In another example, a daily dose of the topical composition can be from about 25 ul to about 10 ml of SOD.

In another example, the method can comprise administering the therapeutically effective amount of the topical composition to the subject 1 to 5 times per day.

In another example, the method can comprise administering the therapeutically effective amount of the topical composition to the subject according to a dosage regimen of at least once per day for a duration of from about a single day to about 3 months.

In another example, symptoms of the condition can be reduced by at least 10% since commencement of the treatment.

In another example, a method of stabilizing a superoxide dismutase (SOD) can comprise: combining an amount of SOD with deoxygenated water to form an SOD solution; and minimizing exposure of the SOD solution to reactive oxygen species (ROS).

In another example, the deoxygenated water can have less than one or more of: about 10 ppm of oxygen, about 5 ppm of oxygen, about 3 ppm of oxygen, about 1 ppm of oxygen, or combinations thereof.

In another example, the SOD can be present in the SOD solution at a concentration of from about 0.0001 wt% to about 10 wt%.

In another example,when the SOD is present in the SOD solution at a concentration of 0.02 wt%: more than about 80% of the SOD can remain in a reactive form after 2 weeks when stored at room temperature and 75% relative humidity; or more than about 80% of the SOD can remain in a reactive form after 4 weeks when stored at room temperature and 75% relative humidity; or more than about 80% of the SOD can remain in a reactive form after 2 weeks when stored at 40° C. and 75% relative humidity; or more than about 80% of the SOD can remain in a reactive form after 4 weeks when stored at 40° C. and 75% relative humidity.

In another example, when the SOD is present in the SOD solution at a concentration of 0.02 wt%: more than about 90% of the SOD can remain in a reactive form after 2 weeks when stored at room temperature and 75% relative humidity; or more than about 90% of the SOD can remain in a reactive form after 4 weeks when stored at room temperature and 75% relative humidity; or more than about 90% of the SOD can remain in a reactive form after 2 weeks when stored at 40° C. and 75% relative humidity; or more than about 90% of the SOD can remain in a reactive form after 4 weeks when stored at 40° C. and 75% relative humidity.

In another example, more than about 80% of the SOD can remain in a reactive form after 365 days when stored at room temperature and 75% relative humidity.

In another example, the SOD solution can further comprise lidocaine.

In another example, when the SOD is present in the SOD solution at a concentration of 0.02 wt% and the lidocaine is present in the SOD solution at a concentration of 0.5 wt%: more than about 80% of the SOD can remain in a reactive form after 2 weeks when stored at room temperature and 75% relative humidity; or more than about 80% of the SOD can remain in a reactive form after 4 weeks when stored at room temperature and 75% relative humidity; or more than about 80% of the SOD can remain in a reactive form after 2 weeks when stored at 40° C. and 75% relative humidity; or more than about 80% of the SOD can remain in a reactive form after 4 weeks when stored at 40° C. and 75% relative humidity.

In another example, when the SOD is present in the SOD solution at a concentration of 0.02 wt% and the lidocaine is present in the SOD solution at a concentration of 0.5 wt%: more than about 90% of the SOD can remain in a reactive form after 2 weeks when stored at room temperature and 75% relative humidity; or more than about 90% of the SOD can remain in a reactive form after 4 weeks when stored at room temperature and 75% relative humidity; or more than about 90% of the SOD can remain in a reactive form after 2 weeks when stored at 40° C. and 75% relative humidity; or more than about 90% of the SOD can remain in a reactive form after 4 weeks when stored at 40° C. and 75% relative humidity.

In another example, the SOD solution can be substantially free of ethanol, capsaicin, menthol, carbomer, and combinations thereof.

In another example, the SOD can be one or more of human recombinant SOD (rhSOD), SOD-1, SOD-2, SOD-3, or a combination thereof.

In another example, a topical composition for use in the treatment of a condition in a subject that is responsive to treatment with superoxide dismutase can comprise: administering a therapeutically effective amount of the topical composition to the subject.

In another example, the condition can be pain.

In another example, the pain ca nbe one or more of chronic pain, back pain, leg pain, ankle pain, joint pain, arthritic pain, wound pain, or combinations thereof.

In another example, the condition can be edema.

In another example, the condition can be inflammation.

In another example, the condition can be erectile dysfunction.

In another example, the condition can be interstitial cystitis.

In another example, the use of a topical composition in the manufacture of a medicament for the treatment of a condition in a subject that is responsive to treatment with superoxide dismutase can comprise: administering a therapeutically effective amount of the topical composition to the subject.

In another example, the condition can be pain.

In another example, the pain can be one or more of chronic pain, back pain, leg pain, ankle pain, joint pain, arthritic pain, wound pain, or combinations thereof.

In another example, the condition can be edema.

In another example, the condition can be inflammation.

In another example, the condition can be erectile dysfunction.

In another example, the condition can be interstitial cystitis.

### EXPERIMENTAL EXAMPLES

The following examples are provided to promote a more clear understanding of certain embodiments of the present disclosure, and are in no way meant as a limitation thereon.

### Example 1 – SOD Composition

### Methods

Preparation of aqueous solution: 20 mM phosphate buffer is prepared at pH 5.5. Various concentrations of SOD are dissolved in water, and the dissolved oxygen (O₂) in the water is removed by decompression and the residual oxygen (O₂) is removed by an inert gas (e.g., nitrogen (N₂)) until the dissolved oxygen content is reduced to 2 ppm.

Preparation of stabilizing agent solution: The stabilizing agent (e.g., soybean lecithin) is dissolved with tocopherol and acrylic resin L100 with a suitable volume of anhydrous ethanol.

Preparation of resultant solution: The stabilizing agent solution and the aqueous solution are combined by peristaltic pump. The ethanol is removed by evaporation. The resultant composition is mixed well and homogenized to form a liposomal solution with a suitable particle size. The resultant composition can be used immediately or can be stored in an environment substantially free of oxidation.

### Results

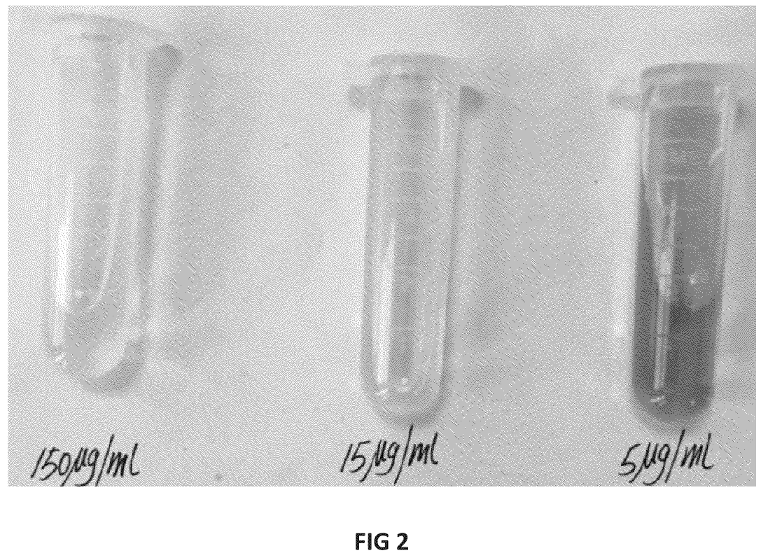

SOD activity test: The activity of encapsulated SOD was tested by a SOD activity assay kit. The reaction system of xanthine and xanthine oxidase was used to produce superoxide anion radical (O2⁻), which oxidized hydroxylamine to form nitrite, and formed purple color under the action of chromogenic agent. The absorbance was detected by visible light spectrophotometer. When active SOD is contained in the tested sample, it has a specific inhibitory effect on superoxide anion free radical ions, so as to reduce the formed nitrite. The absorbance value of the measured sample is lower than that of the control sample under the colorimetric condition. The SOD activity in the tested sample was calculated accordingly. Here, liposomes containing different concentration of SOD (5 µg/mL, 15 µg/mL and 150 µg/mL) were formulated, and SOD activity was tested respectively, as shown in Table I, and as illustrated in FIG. 2.

TABLE I

| SOD Concentration (pg/mL) | Enzyme activity before encapsulation in liposome (U/mL) | Enzyme activity after encapsulation in liposome (U/mL) |
|---|---|---|
| 5 | 40-50 | Unable to detect |
| 15 | 130-160 | -13 |
| 150 | 1400-1700 | -150 |

According to the enzyme activity results, the activity retention rate of SOD enzyme after preparation was about 10%. The higher concentration of the enzyme, the higher remaining activity after the preparation of liposomes. Considering the cost of the enzyme and the loss of activity during preparation and storage, SOD at a concentration of 150 µg/mL was used in liposome preparation.

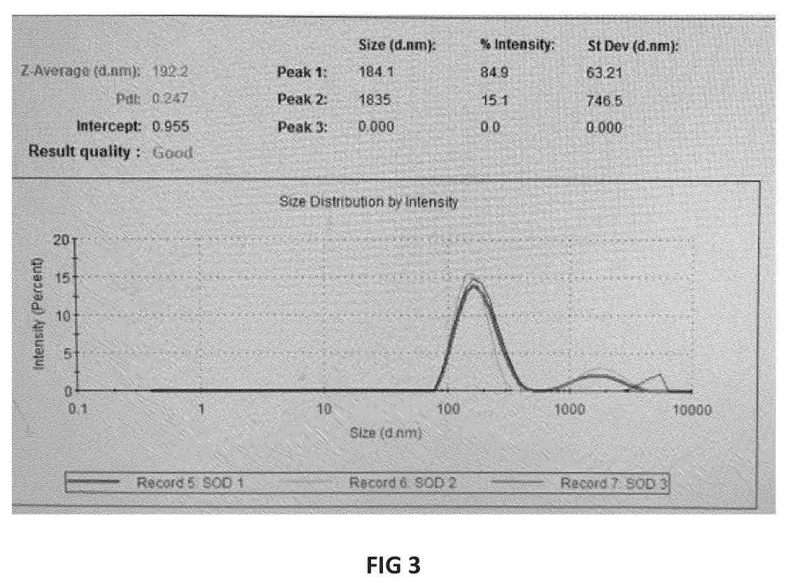

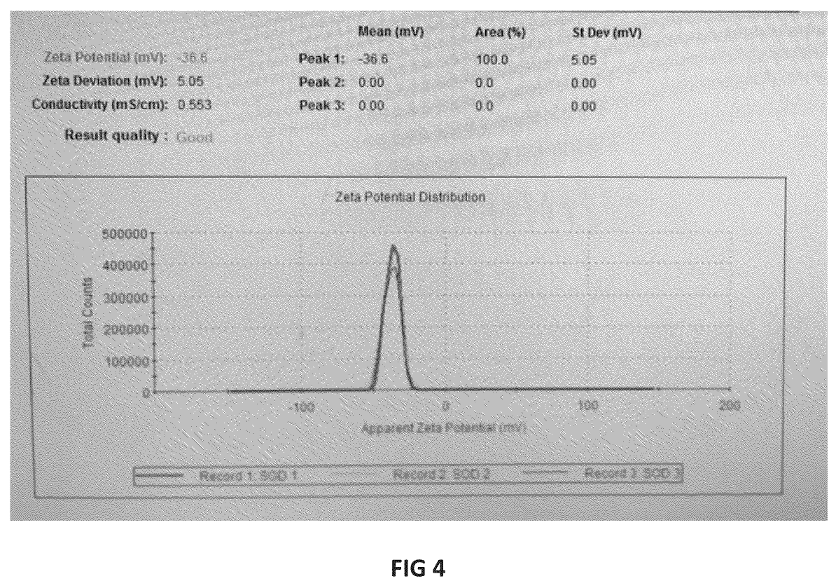

Particle size and zeta potential detection: Particle size and zeta potential was detected by Malvern particle size analyzer (Malvern Panalytical, UK) following the standard protocol. The average liposome particle size is 192.2 nm, as shown in FIG. 3. with a dispersion coefficient PDI value at 0.247; and the zeta potential is -36.6 mV, as shown in FIG. 4.

Conclusions: SOD was formulated in an encapsulated liposome. With the activity detection, SOD at a concentration of 150 µg/mL was used in liposome preparation, and 10% activity remained after liposome generation. The average liposome particle size was 192.2 nm with a dispersion coefficient PDI value at 0.247, and a zeta potential at -36.6 mV.

### Examples 2-5– OD Compositions

An SOD containing composition is prepared by using the components set forth in Table II.

Preparation of aqueous solution: SOD formulations for transdermal delivery of Examples 2-5 can include the components as set forth in Table II. 20 mM phosphate buffer is prepared at pH 5.5. Various concentrations of SOD are dissolved in water, and the dissolved oxygen (O₂) in the water is removed by decompression and the residual oxygen (O₂) is removed by an inert gas (e.g., nitrogen (N₂)) until the dissolved oxygen content is reduced to 2 ppm. Emulsifiers are added until completely dissolved. Penetration enhancers are added and mixed thoroughly.

Preparation of stabilizing agent solution: The stabilizing agent (e.g., soybean lecithin) is dissolved with tocopherol and acrylic resin L100 with a suitable volume of anhydrous ethanol.

Preparation of resultant solution: The stabilizing agent solution and the aqueous solution are combined by peristaltic pump. The ethanol is removed by evaporation. The resultant composition is mixed well and homogenized to form a liposomal solution with a suitable particle size. The resultant composition can be used immediately or can be stored in an environment substantially free of oxidation.

TABLE II

| Components/ Attributes | Weight % | | | |
|---|---|---|---|---|
| | Example 2 | Example 3 | Example 4 | Example 5 |
| SOD | 0.00001 | 0.001 | 0.01 | 1 |
| Stabilizing Agent (e.g., lecithin) | 1 | 2 | 3 | 5 |
| Penetration Enhancer (e.g. Propylene Glycol) | 3% | 3% | 3% | 3% |
| Mineral oil | 1% | 1% | 1% | 1% |
| Emulsifier | 1% | 1% | 1% | 1% |
| Phosphate Buffer | q. s. to adjust pH 5.5 | q.s. to adjust pH 5.5 | q. s. to adjust pH 5.5 | q.s. to adjust pH 5.5 |
| Water for injection | q.s. | q. s. | q. s. | q. s. |

### Example 6 – SOD Composition

An SOD containing composition is prepared by using the components set forth in Table III.

Preparation of aqueous solution: 20 mM phosphate buffer is prepared at pH 5.5. Various concentrations of SOD are dissolved in water, and the dissolved oxygen (O₂) in the water is removed by decompression and the residual oxygen (O₂) is removed by an inert gas (e.g., nitrogen (N₂)) until the dissolved oxygen content is reduced to 2 ppm.

Preparation of stabilizing agent solution: The stabilizing agent (e.g., soybean lecithin) is dissolved with tocopherol with a suitable volume of anhydrous ethanol.

Preparation of resultant solution: The stabilizing agent solution and the aqueous solution are combined by peristaltic pump. The ethanol is removed by evaporation. The resultant composition is mixed well and homogenized to form a liposomal solution with a suitable particle size. The resultant composition can be used immediately or can be stored in an environment substantially free of oxidation.

Capsule formation: A predetermined quantity of the fill material is disposed into a capsule (for example, hard gelatin capsule) to get the SOD dose per dosage unit. The capsules are allowed to cool at room temperature, banded (if required) and packaged in a HDPE bottle and tightly closed with an appropriate lid.

TABLE III

| | |
|---|---|
| Example 6 | Composition mg/capsule |
| SOD | 5-200 mg |
| Stabilizing agent (e.g., lecithin) | 0.05-20 mg |

### Example 7 - SOD Composition

An SOD containing composition is prepared similarly to the method described in Example 1 using the components set forth in Table IV.

TABLE IV

| | |
|---|---|
| Example 7 | Composition mg/capsule |
| SOD | 750 µg |
| Stabilizing agent (e.g., lecithin) | 0.05-20 mg |
| Solidifying agent (e.g. polyethylene glycol, 8000 or PEG 8000) | 0.05-20 mg |

### Example 8-10 – SOD Composition

An SOD containing composition is prepared for liquid oral delivery by using the components set forth in Table VI.

Preparation of aqueous solution: 20 mM phosphate buffer is prepared at pH 5.5. Various concentrations of SOD are dissolved in water, and the dissolved oxygen (O₂) in the water is removed by decompression and the residual oxygen (O₂) is removed by an inert gas (e.g., nitrogen (N₂)) until the dissolved oxygen content is reduced to 2 ppm.

Preparation of stabilizing agent solution: The stabilizing agent (e.g., soybean lecithin) is dissolved with tocopherol with a suitable volume of anhydrous ethanol.

Preparation of resultant solution: The stabilizing agent solution and the aqueous solution are combined by peristaltic pump. The ethanol is removed by evaporation. The resultant composition is mixed well and homogenized to form a liposomal solution with a suitable particle size. The resultant composition can be used immediately or can be stored in an environment substantially free of oxidation.

TABLE V

| Components/ Attributes | Weight % | | |
|---|---|---|---|
| | Example 8 | Example 9 | Example 10 |
| SOD | 0.001 | 0.01 | 0.1 |
| Stabilizing agent (e.g., lecithin) | 10 | 10 | 10 |

### Example 11 – SOD Composition

Preparation of aqueous solution: SOD formulations for parenteral delivery can include the components as set forth in Table VI. 20 mM phosphate buffer is prepared at pH 5.5. The tonicity agent can be added to get a clear solution. Various concentrations of SOD are dissolved in water, and the dissolved oxygen (O₂) in the water is removed by decompression and the residual oxygen (O₂) is removed by an inert gas (e.g., nitrogen (N₂)) until the dissolved oxygen content is reduced to 2 ppm.

Preparation of stabilizing agent solution: The stabilizing agent (e.g., soybean lecithin) is dissolved with tocopherol with a suitable volume of anhydrous ethanol.

Preparation of resulting solution: The stabilizing agent solution and the aqueous solution are combined by peristaltic pump. The ethanol is removed by evaporation. The resultant solution is mixed well and homogenized to form a liposomal solution with a suitable particle size. Make up the volume of the solution to the batch size with water for injection. The inert gas bubbling is continued throughout the solution preparation. The resultant composition can be used immediately or can be stored in an environment substantially free of oxidation. Filter the solution using PVDF filter, and fill the filtered aqueous composition into 100 mL glass vials at room temperature. Before stoppering the containers, layer inert gas over the aqueous composition to reduce the head space oxygen to less than 4% and seal them with appropriate seals. The sealed containers are sterilized in an autoclave at 121° C. for 30 minutes.

TABLE VI

| | | | | |
|---|---|---|---|---|
| Example 11 | Formulation 11a | Formulation 11b | Formulation 11c | Formulation lid |
| SOD | 15 mcg/mL | 250 mcg/mL | 725 mcg/mL | 1500 mcg/mL |
| Stabilizing agent (e.g., lecithin) | 0.01 mg/mL | 0.02 mg/mL | 0.03 mg/mL | 0.05 mg/mL |
| Tonicity Agent (e.g., mannitol) | 20.0 mg/mL | 20.0 mg/mL | 20.0 mg/mL | 20.0 mg/mL |
| Water for injection | q. s. to 1 mL | q. s. to 1 mL | q.s. to 1 mL | q.s. to 1 mL |
| Phosphate Buffer | q. s. to adjust pH 5.5 | q.s. to adjust pH 5.5 | q. s. to adjust pH 5.5 | q. s. to adjust pH 5.5 |

### Example 12-A - Stability of SOD Activity in Sample Formulations (2 Weeks)

The stability of SOD1 was measured for the formulations presented in Table VII. SOD-1 activity testing was conducted at T = 0 and T= 2 weeks for controlled room temperature and for 40° C. A positive and negative sample were also run (e.g., Batch F and Batch G). The SOD activity was measured in terms of U/mL on a scale from 0 to 1000, with 0 indicating no SOD-1 activity and 1000 indicating full SOD-1 activity.

TABLE VII

| Batch | Description | Condition | T=0 SOD Activity (U/mL) (0-1000) | T = 2 Weeks SOD Activity (U/mL) (0-1000) |
|---|---|---|---|---|
| A-1 | 0.02% w/w SOD-1 Gel (with 30% Ethanol) | CRT | 720 | 898 |
| A-2 | 0.02% w/w SOD-1 Gel (with 30% Ethanol) | 40° C. | 720 | 545 |
| B-1 | 0.02% w/w SOD-1 Gel (with 0.5% Lidocaine and 30% Ethanol) | CRT | 790 | 885 |
| B-2 | 0.02% w/w SOD-1 Gel (with 0.5% Lidocaine and 30% Ethanol) | 40° C. | 790 | 584 |
| C-1 | 0.02% w/w SOD-1 Gel (with 0.5% Lidocaine and 0% Ethanol) | CRT | 836 | 1006 |
| C-2 | 0.02% w/w SOD-1 Gel (with 0.5% Lidocaine and 0% Ethanol) | 40° C. | 836 | 938 |
| D-1 | 0.02% w/w SOD-1 Gel (with 0% Lidocaine and 0% Ethanol) | CRT | 818 | 988 |
| D-2 | 0.02% w/w SOD-1 Gel (with 0% Lidocaine and 0% Ethanol) | 40° C. | 818 | 929 |
| E-1 | 0.02% w/w SOD-1 Gel (with 0.025% Capsaicin and 30% Ethanol) | CRT | 791 | 908 |
| E-2 | 0.02% w/w SOD-1 Gel (with 0.025% Capsaicin and 30% Ethanol) | 40° C. | 791 | 68 |
| F* | 0.02% w/w SOD-1 Solution (Positive Control Sample; Deoxygenated Water) | 5° C. | 720 | 958 |
| G** | 0.02% w/w SOD-1 Solution (Negative Control Sample; Deoxygenated Water; pH 4.5) | 5° C. | 58 | 25 |

CRT = Controlled room temperature
* Batch F initial interval at 120 days; T = 2 weeks at 134 days.
**Batch G initial interval at 179 days, T = 2 weeks at 193 days.

The results indicate that ethanol and capsaicin have a significant degradative effect on SOD-1 stability in 2 weeks, as shown for Batches A-1, A-2, B-1, B-2, E-1, and E-2. In contrast, Batches C-1 and C-2 indicate that SOD-1 is stable in a gel formulation after 2 weeks in the presence of lidocaine. Furthermore, Batches D-1 and D-2 indicate that SOD-1 is stable in a gel formulation when lidocaine is absent.

### Example 12-B – Stability of SOD Activity in Sample Formulations (23 Days)

The stability of SOD1 was measured for the formulations presented in Table VIII. SOD-1 activity testing was conducted at T = 0 days and T= 23 days for controlled room temperature and for 40° C. A positive and negative sample were also run (e.g., Batch F and Batch G). The SOD activity was measured in terms of U/mL on a scale from 0 to 1000, with 0 indicating no SOD-1 activity and 1000 indicating full SOD-1 activity.

TABLE VIII

| Batch | Description | Condition | T=0 SOD Activity (U/mL) (0-1000) | T = 23 Davs SOD Activity (U/mL) (0-1000) |
|---|---|---|---|---|
| C-1 | 0.02% w/w SOD-1 Gel (with 0.5% Lidocaine and 0% Ethanol) | CRT | 836 | 842 |
| C-2 | 0.02% w/w SOD-1 Gel (with 0.5% Lidocaine and 0% Ethanol) | 40° C. | 836 | 744 |
| D-1 | 0.02% w/w SOD-1 Gel (with 0% Lidocaine and 0% Ethanol) | CRT | 818 | 831 |
| D-2 | 0.02% w/w SOD-1 Gel (with 0% Lidocaine and 0% Ethanol) | 40° C. | 818 | 804 |
| F* | 0.02% w/w SOD-1 Solution (Positive Control Sample; Deoxygenated Water) | 5° C. | 720 | 776 |
| G** | 0.02% w/w SOD-1 Solution (Negative Control Sample; Deoxygenated Water; pH 4.5) | 5° C. | 58 | 60 |

CRT = Controlled room temperature
* Batch F initial interval at 120 days; T = 23 days at 143 days.
**Batch G initial interval at 179 days, T = 23 days at 202 days;

The results indicate that SOD-1 is stable in a gel formulation after 23 days in the presence of lidocaine (Batches C-1 and C-2). Furthermore, Batches D-1 and D-2 indicate that SOD-1 is stable after 23 days in a gel formulation when lidocaine is absent.

### Example 13 – Manufacturing Instructions for rh-SOD 1 Gel Formulations

Phosphate Buffer (pH 7.4) was made using the following formula and processing operations, as shown in Table IX.

TABLE IX

Preparation of Phosphate Buffer, pH 7.4 (batch size of 200 ₂).

| | | | |
|---|---|---|---|
| Phase | Ingredient | Concentration (%w/w) | Target Amount (g) |
| A | Sterile Water for Irrigation | 95.00 | 190.00 |
| A | Dibasic Sodium Phosphate | 1.01 | 2.02 |
| A | Monobasic Sodium Phosphate | 0.169 | 0.338 |
| B | 10% Hydrochloric Acid | pH to 7.4 | As Used. |
| B | IN Sodium Hydroxide | pH to 7.4 | As Used. |
| C | Sterile Water for Irrigation | Q.S. to 100% | As Used. |

1. Add Phase A ingredients into the main vessel. Mix until the powders are completely dissolved.
2. Adjust the pH of the batch with Phase B to 7.4.
3. Q.S. the batch to 100% with Phase C.

Just before the manufacturing of the gels, the Phosphate Buffer (pH 7.4) is degassed using one of the following two procedures.

### Nitrogen Gas Bubbling Method:

1. Place a piece of tubing such that one end is connected securely to the Nitrogen Gas tank manifold and the other is just touching the bottom of the main vessel.

2. Slowly turn on the Nitrogen Gas such that there is enough pressure to gently bubble the Phosphate Buffer. Cover the main vessel to prevent splashing.

3. Continue bubbling for no less than 30 minutes.

4. Measure and record the dissolved oxygen of the Phosphate Buffer with a dissolved oxygen meter. Acceptable dissolved oxygen values are ≤5.00 mg/L.

### Vacuum Chamber Method:

1. Place the vacuum chamber so that it is centered over a hot/stir plate. Place the uncovered main vessel, along with a magnetic stir bar, in the center of the vacuum chamber.

2. Turn on the mixing setting to 300 RPM and begin heating the Phosphate Buffer until it reaches 30° C. Turn down the heat setting to maintain this temperature.

3. Cover the vacuum chamber with its associated lid. With mixing and heating, pull the vacuum to -25 in Hg.

4. Continue degassing for no less than 45 minutes.

5. Measure and record the dissolved oxygen of the Phosphate Buffer with a dissolved oxygen meter. Acceptable dissolved oxygen values are ≤5.00 mg/L.

Below are the formulation compositions and procedures used for the manufacturing at a 100 g scale for 0.02% rh-SOD1 Cellulose Gel without Lidocaine (Table X and with Lidocaine (Table XI).

TABLE X

Preparation of rh-SOD1 Cellulose Gel, 0.02% w/w (w/o Lidocaine) (100 g)

| | | | |
|---|---|---|---|
| Phase | Ingredient | Concentration (%w/w) | Target Amount (g) |
| A | Phosphate Buffer (pH 7.4; Degassed) | 88.48 | 88.48 |
| A | Methylparaben | 0.10 | 0.10 |
| A | Propylparaben | 0.01 | 0.01 |
| B | rh-SOD1 | 0.02 | 0.02 |
| C | Hydroxyethylcellulose (Natrosol 250 HHX PHARM) | 1.00 | 1.00 |
| D | 10% Hydrochloric Acid | pH to 7.4 | As Used. |
| D | 1N Sodium Hydroxide | pH to 7.4 | As Used. |
| E | Phosphate Buffer (pH 7.4; Degassed) | Q.S. to 100% | As Used. |

1. Add Phase A ingredients to the main vessel. Mix until a clear solution is obtained.
2. Add Phase B ingredient to the main vessel. Mix until a clear solution is obtained.
3. Slowly add Phase C ingredient to the main vessel with mixing. Continue mixing until a uniform gel is formed.
4. Adjust the pH of the batch with Phase D to 7.4.
5. Q.S. batch to 100% with Phase E.

TABLE XI

Preparation of rh-SOD1 Cellulose Gel, 0.02% w/w (With Lidocaine 0.5%) (100 g)

| | | | |
|---|---|---|---|
| Phase | Ingredient | Concentration (%w/w) | Target Amount (g) |
| A | Phosphate Buffer (pH 7.4; Degassed) | 78.48 | 78.48 |
| A | Methylparaben | 0.10 | 0.10 |
| A | Propylparaben | 0.01 | 0.01 |
| B | rh-SOD1 | 0.02 | 0.02 |
| C | Phosphate Buffer (pH 7.4; Degassed) | 10.00 | 10.00 |
| C | Lidocaine HCl | 0.50 | 0.50 |
| D | Hydroxyethylcellulose (Natrosol 250 HHX PHARM) | 1.00 | 1.00 |
| E | 10% Hydrochloric Acid | pH to 7.4 | As Used. |
| E | 1 N Sodium Hydroxide | pH to 7.4 | As Used. |
| F | Phosphate Buffer (pH 7.4; Degassed) | Q.S. to 100% | As Used. |

1. Add Phase A ingredients to the main vessel. Mix until a clear solution is obtained.
2. Add Phase B ingredient to the main vessel. Mix until a clear solution is obtained.
3. In a separate vessel, combine Phase C ingredients. Mix until a clear solution is obtained.
4. Slowly add Phase C to the main vessel with mixing. Continue mixing until uniform in appearance.
5. Slowly add Phase D to the main vessel with mixing. Continue mixing until a uniform gel is formed.
6. Adjust the pH of the batch with Phase E to 7.4.
7. Q.S. batch to 100% with Phase F.

### Example 14 – Treatment of ALS

### Case Study 14-1

The subject was a 55-year-old male who had sporadic ALS for about 3 years. The pain he felt was greatest in his shoulders, but his shoulders do not have significant pain unless he is straining his shoulders. The subject was administered the SOD treatment (0.02% SOD in deoxygenated water with liposomes) for two weeks. After the two weeks, the subject had about a 60% pain reduction in his shoulders compared to pre-treatment levels; however, the tremor in one of his hands remained the similar when compared to the tremor before commencing the treatment.

### Case Study 14-2

The subject was an elderly woman with ALS for the previous 2.5 years who had significant neck pain. The subject was administered the SOD treatment (0.02% SOD in deoxygenated water with liposomes). After one day, the subject did not notice any significant improvement.

### Example 15 – Treatment of SOD Genetic Errors

### Case Study 15-1

A woman in her early 40s was diagnosed with SOD genetic errors - two homozygous SOD2 errors and one homozygous SOD3 error. The subject did not appear to be producing the SOD2 and SOD3 enzymes. The subject exhibited brain fog, general weakness, and lower back and hip pain. The subject self-administered 2-3 squirts of the SOD solution (0.02% SOD in deoxygenated water with liposomes) onto her lower back and 2-3 squirts onto her neck. After about 4 minutes, the subject noticed an improvement in mobility in her back and neck compared to pre-treatment. The subject self-administered an additional 2-3 squirts of SOD solution onto her lower back. After about one minute, the subject noticed even more mobility in her lower back compared to pre-treatment levels.

The subject was re-evaluated 2.5 hours later and noticed that the back pain had gone away for a while before coming back. The subject was evaluated about 24 hours after the initial application of treatment. The subject noticed her back loosening up with less tightness. The pain in her back had been reduced from a 3-4 level on a scale from 0 (no pain) to 10 (high pain) to a level of 1-2. One week later, the subject was re-evaluated and had noticed that the pain was mostly gone.

### Case Study 15-2

The subject was a middle-aged woman who had lower back pain of unknown cause. The subject was diagnosed with a SOD3 error. The subject self-administered 4-5 drops of the SOD solution (0.02% SOD in deoxygenated water with liposomes) onto her back – which resulted in the pain being gone within about 30 seconds. The subject self-administered about 1-2 drops of the SOD solution onto her neck, and she noticed that the pain was gone there in about 5 seconds - having been reduced from a pain level of 6 on a scale from 0 (no pain) to 10 (high pain) to a 0.

The subject was evaluated 2 hours later. The subject noticed that the treatment was still effective even though she had not applied any additional SOD solution. The subject was evaluated 24 hours after the initial application of SOD solution. The subject did not apply any more treatment and had felt tightness when shifting in bed but still had increased mobility. The subject was evaluated 1 week after the initial application of SOD solution. The subject had administered some of the SOD solution when she had woken up with pain. The pain had subsided.

### Case Study 15-3

The subject was a middle-aged woman who had been diagnosed with an SOD error. The subject had constant pain in her legs, shoulders, chest, and rib cage. The subject self-administered the SOD solution (0.02% SOD in deoxygenated water with liposomes) to the affected areas. The subject noticed that her ankles felt less pain after about one minute; however, the subject still had aches in her thighs. The subject was evaluated 2.5 hours after the initial application of SOD solution. The subject had self-administered more SOD solution without noticing a difference. The subject was evaluated 24 hours after the initial application of treatment. The subject was less achy but had felt sick otherwise with muscle cramps, nausea, and migraines. The subject was evaluated one week after the initial application of SOD treatment. The subject did not notice that the SOD treatment was effective for her.

### Example 16 – Treatment of Osteoarthritis

### Summary

4 Osteoarthritis cases were examined involving the hands, lower back, and knees. All of these subjects were older (greater than 50 years of age) and had poor results with NSAIDS or opioids and had suffered greatly with pain levels ranging from 2-3 to 7-9 on a scale from 0 (low pain) to 10 (high pain). Most took NSAIDs when in great pain but had noticed little improvement. Subjects were told to apply the SOD onto their most severely inflamed joints whenever they felt pain. Patients generally noted a significant and almost immediate (within a minute or so or even less) improvement in their pain and inflammation, increased affected joint mobility, and improved sleep and sense of well-being. Some commented that they had not felt this well in decades.

### Case Study 16-1

The subject self-administered SOD1 three times per day in a topical solution (0.02% SOD in deoxygenated water with liposomes) in the amount of about 2-3 squirts per administration to the affected area. The subject was a female subject of older age who had osteoarthritis in her hands, feet, and neck. An MRI had confirmed the osteoarthritis on her neck. The pain level of the arthritis was severe in all three areas.

The subject applied topical SOD to the affected area - hands and feet. Before application, the subject exhibited severe pain. About 30 seconds after application of the SOD, the pain was no longer present. The subject also applied topical SOD to her neck with a slight decrease in pain.

After about 24 hours, the subject also exhibited decreased swelling in her knuckles with no side effects over the previous 24 hours. With each application of the SOD to the affected area, the pain subsided within about 30 seconds.

After one week of the initial application, the subject noticed reduced pain and reduced tightness in her neck with application of topical SOD in a creme formulation. Before application the pain was severe and debilitating, but after application, the pain was tolerable.

### Case Study 16-2

The subject had severe osteoarthritic pain on her left hands and wrist. She had received a steroid injection the day before, but that injection had removed about 70-80% of the pain. The pain was still non-stop and increased when the left hand or wrist felt increased pressure. The subject was administered about 3-5 squirts of SOD (0.02% SOD in deoxygenated water with liposomes) on her left wrist and hand. 60 seconds after topical administration, the pain was barely noticeable.

### Case Study 16-3

The subject was a woman in her 50s with pain in joints and fingers that had developed after the past year. The pain in her knees and fingers was at a level of 2 on a scale from 0 (low pain) to 10 (high pain). The subject had meniscus issues in her left knee and had meniscus surgery in her left knee about 2.5 years previously. The subject had pain and inflammation in the affected areas.

The subject topically applied 2-3 drops of SOD solution (0.02% SOD in deoxygenated water with liposomes) on her left knee using her left pinky finger. After about 5 seconds, the subject had noticeable pain relief in her left pinky finger and pain relief in her left knee after about 60 seconds. The subject topically applied 2-3 drops on her right knee using her right pinky finger. After about 5 seconds, the subject’s pain in her right pinky finger had been reduced in half, and her pain relief in her right knee after about 90 seconds with a reduction in pain by about 80%. 15 minutes after application, the subject did not feel any pain in her knees or fingers.

### Case Study 16-4

The subject was a middle-aged female. The subject had a history of pain in her knees. She had previously tried cortisone injections and on occasion fluid-buildup in the knees had been drained off. In her current condition, she relied on diet to control the knee pain. The subject applied about 3 drops of SOD solution (0.02% SOD in deoxygenated water with liposomes) to her knee and still felt a similar amount of pain after about 30 seconds. After about 100 seconds, the pain in that area was significantly lower when measured in a standing position.

### Example 17 – Treatment of Interstitial Cystitis

2 Interstitial Cystitis (IC) Patients were examined. Both subjects each had more than 10 years of suffering from IC. Both were having severe urgency (50-150 times a day), symptoms of severe inflammation of the bladder with pain and stiffness of the anterior bladder wall, and feelings of depression, fatigue, and insomnia. Pain levels for both ranged from 5-10, on a scale from 0 (no pain) to 10 (high pain). The subjects were directed to apply the serum (0.02% SOD in deoxygenated water with liposomes) to their bladder and any place else that hurt (both subjects also suffered from fibromyalgia).

Both subjects, within 24 hours of use, had almost complete resolution of pain, bladder stiffness, inflammation, and then eventual marked improvement to normalcy in their urgency. Both noticed dramatic improvement after application in less than 30 seconds. Eventually both patients applied the serum as needed every 8-12 hours (which became far and fewer applications as time went by and as their inflammation lessened). The subjects noted that it was the first time in years where they’d felt normal.

A third IC subject had a pain level of 3 on a scale from 0 (no pain) to 10 (high pain). The pain was reduced to 1 5 minutes after SOD was applied to the lower abdomen over the bladder.

### Example 18 – Treatment of Fibromyalgia

### Summary

6 Fibromyalgia Subjects were examined. These subjects all had typical fibromyalgia with numerous tender points, chronic fatigue, severe joint/nerve/skin/muscle/tendon pain, and allesthesia that usually ranged from 3-4 up to 8-9 on a pain scale from 0 (low pain) to 10 (high pain) and were often bedridden secondary to their pain and fatigues. Most subjects were on NSAID or narcotics for pain. Fibromyalgia duration was greater than ten years in all participants. Subjects were told to apply the study serum (0.02% SOD in deoxygenated water with liposomes) to their areas of most severe pain as often as they saw fit and to move the application out from there.

Most subjects had anywhere from 50-60% improvement to almost complete resolution of most of their pain issues. Though the serum samples were small (15ml), most subjects in this study were able to stretch the relief out by applying a drop or two of the serum to areas with the most intense pain. The subjects would also use the drops as far apart as they could over 8-12 hours trying to conserve the serum. Sleep for most subjects also dramatically improved and most noted a sense of increased well-being (a common finding throughout all these studies). Many of these patients were able to temporarily reduce their NSAID, pain medication, and opioid use. All subjects wished to continue with their serum applications in lieu of any regular therapies they were on.

### Selected Case Study

The subject was a middle-aged woman who had pain starting in her early 20s. The subject also had asthma and Crohn’s disease. The fibromyalgia intensified through major stress in her feet, arms, right arm, stomach, with her right arm having the worst pain. The subject had pain level of about 4-5 on a scale from 0 (no pain) to 10 (high pain). The subject applied 3-4 drops of the SOD solution (0.02% SOD in deoxygenated water with liposomes) on each area. After about 60-80 seconds, the subject noticed motion more mobility in her arms and shoulders with less pain. After 24 hours, the subject had better mobility. The subject still had some pain on her lower arm and on hips, but the pain and mobility were significantly better otherwise. The subject said her asthma was improving, her pain level was down to about a 3 overall.

### Example 19-A – Treatment of Pain

### Case Study 19A-1

The subject was a middle-aged woman. She had a foot fracture and ligament tear about 8 weeks earlier. She has received an MRI the day before which indicated that the ankle was healing, but she still felt pain. The subject also had generalized arthritic pain. The subject rubbed two spays of the SOD solution (0.02% SOD in deoxygenated water with liposomes) onto her ankle and rubbed the remainder onto her hands. Within 100 seconds of application of the SOD, the pain in the ankle was gone and the ankle had gained a significant increase in mobility.

The subject was examined two hours after application of the SOD. The SOD had additional effects including the clearing up of brain fog, but the pain relief was beginning to wear off. The subject re-applied another two sprays of the SOD onto her ankle. The subject was further examined 24 hours after the initial application of the SOD. She had noticed that the pain relief of the SOD had worn out over the night and she had started feeling it wear off after about 4 hours. The subject further applied the SOD onto the affected ankle. The subject also applied SOD onto her neck, which had recently suffered from whiplash. Within 60 seconds, the effects of the pain relief were noticeable. The subject felt pain relief in the ankle with an increased range of motion. The subject also had pain relief in her neck.

### Case Study 19A-2

The subject was a middle-aged woman. Her pain has been present over the past 1.5 years and has gradually gotten worse over that time. Deterioration of the hip socket and joint. level with a pain level of about 6 on a scale from 0 (low pain) to 10 (high pain). The subject applied 2-3 squirts of the SOD solution (0.02% SOD in deoxygenated water with liposomes) on the front and 2-3 on the side of her hip and rubbed it in. The subject noticed that the pain was reduced 60 seconds later. The subject applied an additional amount to a different area, and, after one minute, the subject noticed that the pain was down to a pain level of about 4.

### Case Study 19A-3

A 25-year-old woman had over 30 facial reconstruction surgeries. Her jaw pain was a 9 out of 10 on a scale from 0 (no pain) to 10 (high pain). Right before the SOD administration, her pain level was a 7. After 2 pumps of SOD solution (0.02% SOD in deoxygenated water with liposomes) was applied to her right jaw, the pain was reduced to a 6 after one minute and to a 1 after 5 minutes.

### Case Study 19A-4

A male subject in his late 40s had a 1-inch laceration on his left thenar eminence of thumb region which was repaired at the ER 2 days prior to the visit. The pain level was 8-9 out of 10 on a scale from 0 (no pain) to 10 (high pain). SOD (0.02% SOD in deoxygenated water with liposomes) was applied to the wound. After 1 minute, the pain level was reduced to a 5 out of 10. After 4 minutes, the pain was reduced to a 1 out of 10.

### Case Study 19A-5

A female subject in her early 50s had nerve pain. The pain level was 7-8 out of 10 on a scale from 0 (no pain) to 10 (high pain). SOD (0.02% SOD in deoxygenated water with liposomes) was applied to the affected area. After 5 minutes, the pain was reduced to a 2 out of 10 in the affected areas.

### Case Study 19A-6

A male subject in his mid 60s had bladder pain after more than 40 separate surgeries on his back, neck, shoulder, and bladder. The subject was one oxycontin and Roxicodone. The pain level was 5 out of 10 on a scale from 0 (no pain) to 10 (high pain). SOD (0.02% SOD in deoxygenated water with liposomes) was applied to the area of the bladder. After 5 minutes, the pain was reduced to a 0 out of 10 in the bladder area.

### Case Study 19A-7

A male subject in his mid 60s had severe acute pain in the 1^{st} metatarsal region consistent with gout but the uric acid level was within a normal level. The pain level was 8 out of 10 on a scale from 0 (no pain) to 10 (high pain). SOD (0.02% SOD in deoxygenated water with liposomes) was applied. After 5 minutes, the pain was reduced to a 0 out of 10 in the affected area.

### Case Study 19A-8

A male subject in his mid 50s had a warm right 5^{th} finger joint after a rose thorn had lodged in the area. The pain level was 7 out of 10 on a scale from 0 (no pain) to 10 (high pain) with mild swelling and redness. SOD (0.02% SOD in deoxygenated water with liposomes) was applied. After 1 minute, the pain was reduced to a 7 out of 10 in the affected area. After 5 minutes, the pain was reduced to a 4 out of 10. The subject had also applied a frankincense and silver product. Within 2 days, the redness, warmth, and pain had completely resolved.

### Case Study 19A-9

A female subject in her late 40s had wrist tendonitis with a pain level that was 6 out of 10 on a scale from 0 (no pain) to 10 (high pain). SOD (0.02% SOD in deoxygenated water with liposomes) was applied. After 1 minute, the pain was reduced to a 5 out of 10 in the affected area. After 5 minutes, the pain was reduced to a 0 out of 10 for the first time in the previous 6 months.

### Case Study 19A-10

A male subject in his mid 50s had shoulder pain with a pain level that was 3 out of 10 on a scale from 0 (no pain) to 10 (high pain) when at rest and a 6 out of 10 with movement across the chest. SOD (0.02% SOD in deoxygenated water with liposomes) was applied. After 1 minute, the pain was reduced to a 1 out of 10 in the affected area at rest but stayed at 6 with movement across the chest. After 5 minutes, the pain was reduced to a 0 out of 10 at rest but stayed at 6 with movement across the chest.

### Example 19-B – Treatment of Back Pain

### Case Study 19B-1

The subject was a woman in her early 40s. The subject had pain in her lower back and about a pain level of 5 upon waking on a scale from 0 (low pain) to 10 (high pain), a pain level of 7-8 while sitting, and varies throughout the day. Within 60 seconds of application of SOD (0.02% SOD in deoxygenated water with liposomes) to her lower back the pain level was reduced to a pain level of 0.

### Case Study 19B-2

A male subject in his mid 60s had back pain at pain level of 7 out of 10 on a scale from 0 (no pain) to 10 (high pain). SOD (0.02% SOD in deoxygenated water with liposomes) was applied to the back. After 1 minute, the pain level was reduced to a 6 out of 10. After 5 minutes, the pain was reduced to a 3 out of 10. The subject was not able to bend over before the treatment but was able to bend over after the treatment.

### Case Study 19B-3

A female subject in her late 30s had coccyx pain and was taking Subutex for pain management. The pain level was 8 out of 10 on a scale from 0 (no pain) to 10 (high pain). SOD (0.02% SOD in deoxygenated water with liposomes) was applied to the affected area. After 5 minutes, the pain was reduced to a 0 out of 10.

### Case Study 19B-4

A female subject in her early 30s had right lower back pain that flared up from lifting weights with a pain level that was 6 out of 10 on a scale from 0 (no pain) to 10 (high pain). SOD (0.02% SOD in deoxygenated water with liposomes) was applied. After 1 minute, the pain was reduced to a 5 out of 10 in the affected area. After 5 minutes, the pain was reduced to a 4 out of 10.

### Case Study 19B-5

A female subject in her late 50s had back pain with a pain level that was 5 out of 10 on a scale from 0 (no pain) to 10 (high pain). SOD (0.02% SOD in deoxygenated water with liposomes) was applied. After 1 minute, the pain was reduced to a 4 out of 10 in the affected area. After 5 minutes, the pain was reduced to a 3 out of 10.

### Case Study 19B-6

A female subject in her early 40s had back pain and long history of narcotic use which was weaned down. The subject had a pain level that was 8 out of 10 on a scale from 0 (no pain) to 10 (high pain). SOD (0.02% SOD in deoxygenated water with liposomes) was applied to the lower back. After 2 minutes, the pain had gone away.

### Example 19-C – Treatment of Leg Pain

### Case Study 19C-1

Morel-Lavallee cases are uncommon but severe, tend to be highly traumatizing, for the most part untreatable, usually tend to get infected, and rarely, if ever, heal in any adequate way. This patient was a 56-year-old female woman who was in very good health prior to her accident, when her accelerator on her Harley motorcycle (she was a weekend rider) became stuck open at a gas station slamming her right thigh into a pump. This trauma caused a separation of her anterior thigh muscles from her bone leading to a massive inflow of serous fluid with severe pain. The skin was not broken due to riding leathers she had on. After evaluation by a trauma orthopedist who made the diagnosis (via MRI, careful examinations, CAT scan), she was informed of the diagnosis and the grim outlook (e.g., rarely heal in less than a year, are wholly inadequate when they do heal, and are incredibly debilitating). He prescribed an ice compression machine, advised minimal ambulation, opioids for pain, and cautious physical therapy for the next year.

The subject was directed to apply topical glutathione and SOD as many times a day as she felt the need. The subject showed marked improvement in the swelling, loose fluid in the thigh, a 90-98% improvement in the pain and increased mobility within the first week. Six weeks after commencing treatment, she continued to improve dramatically with improvement in all aspects.

### Case Study 19C-2

A male subject in his mid 20s was hit by a slow-moving car on the back of his leg. The flexion and extension were about 60 percent of normal in the affected leg. The pain level was 6 out of 10 on a scale from 0 (no pain) to 10 (high pain). SOD (0.02% SOD in deoxygenated water with liposomes) was applied. After 5 minutes, there did not appear to be a reduction in pain. One week later, the knee was normal with no pain.

### Example 19-D – Treatment of Ankle Pain

### Case Study 19D-1

The subject was a 28-year-old male who had topically applied SOD (0.02% SOD in deoxygenated water with liposomes) for ankle pain. The ankle was aggravated about 2 weeks earlier when he came down on it. About 2 hours after application, he had a reduction in pain of about 50% without aches or pains. He was able to walk more fluidly, and his ankle had increased mobility.

The subject re-applied the topical SOD in the evening and then woke up with the pain fully back by the morning. The subject re-applied the topical SOD in the morning and noticed a 50% reduction in pain about two hours later. He topically applied more SOD and about two hours later, the pain in his ankle was down about 60% when compared to the morning.

The subject was re-evaluated one week after the initial administration of topical SOD. He had tried the topical SOD for his back pain and sciatic nerve pain without substantial results. The subject also tried the topical SOD for some extreme shoulder pain without substantial results. The subject continued to notice a reduction in pain for his ankle and wrist.

### Case Study 19D-2

The subject was a female in her early 20s. She had broken her ankle about 6 hours earlier and there was a large amount of swelling on the outer part of the ankle. The injury was an acute lower tibia-fibula fracture that was severe in nature. Pain was noted to be a 9-10 on a scale from 0 (low pain) to 10 (high pain), and swelling was extreme. The subject topically applied SOD (0.02% SOD in deoxygenated water with liposomes) to the affected area and noticed significant pain relief and increased mobility. After about 30 minutes, the swelling in the ankle had decreased by about 90%. Results were notable in that the swelling almost completely resided within 3 minutes (enough to clearly see it was a lower tib-fib fracture) and the pain went from a 9-10 to almost non-existent in less than 2 minutes. Before and after surgery the patient used the study serum over

ski opioid she was offered. The orthopedist physician noted that the subject had extremely rapid healing of the lesions with no scarring noted on x-rays on skin. She was ambulating with complete healing in approximately six weeks.

### Case Study 19D-3

A female in her early 40s had a 15+ history of left-sided lateral ankle pain after a fall from an 8-foot height. The subject was on Percocet and Ultram for the pain. Her pain level was a 7 out of 10 on a scale from 0 (no pain) to 10 (high pain). The SOD solution (0.02% SOD in deoxygenated water with liposomes) was applied to the affected area. After 1 minute the pain was reduced to a pain level of 6. After 5 minutes, the pain was reduced to a pain level of 3.

### Case Study 19D-4

A male subject in his late 50s had back pain with a pain level that was 8 out of 10 on a scale from 0 (no pain) to 10 (high pain). The subject had broken his ankle after falling out a truck 20 years before. The subject was taking Roxicodone 7 times per day for the previous 18 years. The SOD (0.02% SOD in deoxygenated water with liposomes) was applied, and the pain was reduced to a 6 out of 10.

### Example 19-E – Treatment of Joint Pain

The subject self-administered rGSH and SOD (0.02% SOD in deoxygenated water with liposomes) twice per day in a topical solution in the amount of about 1-2 sprays per administration to the affected area. The male subject in his 30s had joint pain and tendon pain. A urine test had showed elevated levels of oxidative stress that might have been caused by an antibiotic the subject had taken, ciprofloxacin. An ultrasound has shown tendonitis in the quad tendon. The subject applied topical glutathione (rGSH) and topical SOD with a one-minute interval in between each application. For each application, the subject applied 1-2 sprays on the affected area - the left knee, Achilles, front part of the foot, quad tendon, and ankle. Before application of either the rGSH or the SOD, the subject exhibited pain of 4 (on a scale from 1 to 10). About 5 minutes after application of the SOD, the pain had subsided down to 1.

### Example 20-A – Treatment of Neuropathy

### Summary

These seven cases involved CIPN cases (Chemotherapy Induced Peripheral Neuropathy), diabetic cases, and idiopathic neuropathy cases. None of these subjects has success with previous treatment. A few of the subjects were on gabapentin and many took opioids when the neuropathic pain was more severe. Pain levels could reach up to 6-10 at times. The pain generally tended to be much worse at night. Subjects were advised to place the study serum on the distal ends of the involved extremities as many times a day as they deemed appropriate. All subjects had an improvement in pain, some to the point of resolution. After about 2-3 weeks, all subjects had pain resolution and neuropathy improvement of about 30-95%.

### Case Study 20A-1

A woman had idiopathic peripheral neuropathy in her feet with symptoms including numbness, burning, muscle spasms, aches over the past ten years. The subject was directed to apply topically apply a few drops of SOD solution (0.02% SOD in deoxygenated water with liposomes) and rub it in to her left foot and right foot. After three minutes, the subject a reduction in symptoms. After 24 hours of treatment, the subject noticed increased sensitivity at the top of her feet. After one week of treatment, she was able to reduce her gabapentin from three times per day to once per day while still noticing a reduction in symptoms.

### Case Study 20A-2

The subject applied 6 squirts of SOD (0.02% SOD in deoxygenated water with liposomes) to the lower front right leg and top part of foot. The subject had felt burning in that area in the morning. 30 seconds after application, the pain was reduced in severity by about 30. About 2 minutes after the initial application, the subject noticed that the surface area of the affected area had decreased by about 70%. After about 5 minutes, the surface area of the affected area had been reduced to about 10% compared to the affected area before treatment had begun. The subject’s overall pain level went down from a 4 to a 3 on a scale of 0 (no pain) to 10 (high pain).

### Case Study 20A-3

The subject was a woman in her 20s who had begun noticing symptoms of neuropathy in her feet about two years earlier. Her overall pain level was usually about a 6 on a scale from 0 (no pain) to 10 (high pain) but would often shoot up to 10. The subject applied SOD (0.02% SOD in deoxygenated water with liposomes) to the feet and noticed that they were not hurting after about 30 seconds, although the numbness was still there. After about 1 hour, she noticed that the pain came back. The subject applied more treatment to the affected area and the pain stopped within 30-60 seconds. After one week of treatment, the subject had been applying the treatment once before nighttime and was able to sleep throughout the night without pain.

### Example 20-B – Treatment of CIPN

The subject had been afflicted with CIPN after having been treated for breast cancer. The pain was present in her feet up to her knees. Her feet were constantly tingling with pins and needles. On a bad day the pain level was about a 6 on a scale from 0 (no pain) to 10 (high pain). On an average day, the pain level was a 2. The subject was directed to administer SOD (0.02% SOD in deoxygenated water with liposomes) to the affected area. She applied 5 pumps to her left leg, top part of left foot, and bottom part of left foot. She noticed a difference after about 15-30 with the reduction in tingling in her feet.

### Example 20-C – Treatment of Diabetic Neuropathy

The subject has a pain level of 10 on a scale from 0 (no pain) to 10 (high pain). After application of treatment (0.02% SOD in deoxygenated water with liposomes), the pain level was reduced to a 7 after about 10 minutes.

### Example 20-D – Treatment of Arthritic Neuropathy

The subject was a woman of age 54. She would often feel a shooting/stabling pain in her feet. The subject was administered SOD (0.02% SOD in deoxygenated water with liposomes) next with 3 total pumps onto the top part of the foot. After about one minute, the subject stood up to put weight on the foot and noticed that the pain was gone. After about 24 hours of treatment, the subject did not have pain on the foot on which the treatment was applied, which was significantly different from her other foot which did not receive treatment.

### Example 20-E – Treatment of Arthritic Osteoarthritic Neuropathy

The subject had open heart surgery about 20 years before which resulted in a nerve bundle through buttocks being compressed. The subject was told that the nerve would be restored over a year, but the nerve was never fully restored. The subject administered SOD (0.02% SOD in deoxygenated water with liposomes) to the nerve damage in the back of leg. After about 90 seconds, the subject noticed that the top of the foot had recovered some sensation with reduced pain.

### Example 21 – Treatment of Shingles

The subject was a male in his early 80s who had begun exhibiting symptoms about 10 days earlier. The subject was administered a gel version of the SOD composition (0.02% SOD in deoxygenated water with liposomes) on his left abdomen and almost halfway at the back to cover the afflicted area. The amount applied was 6 pumps total. The subject’s pain level was a 4 on a scale from 0 (no pain) to 10 (high pain) before treatment. His pain level was reduced to 0 after about one minute after treatment had commenced. After 72 hours, the pain was gone. One week after commencing treatment, the shingles was about 85% reduced in severity over the affected area.

### Example 22 – Treatment of Trigeminal Neuralgia

### Case Study 22-1

The subject was a 70-year-old woman who had trigeminal neuralgia for more than 20 years on the right side of her face. The subject was taking oxcarbazepine to treat the trigeminal neuralgia. The subject self-applied about 1 spray of GSH on the right side of her face. The subject orally swished about 5 sprays of GSH in her mouth around the right side of her face and allowed the GSH to sit in her mouth for about one minute. 5 minutes after the subject had applied the GSH, the subject self-applied 3 separate applications of the SOD (0.02% SOD in deoxygenated water with liposomes) topically on the right side of her face. 90 seconds after the subject had applied the SOD, she noticed a 50% reduction in pain from before treatment had started. 15 minutes after the treatment started, the subject noticed a 75% reduction in pain when compared to the start of treatment.

### Case Study 22-2

The subject was a middle-aged woman who had pain on the right side of her face but was not yet diagnosed as having trigeminal neuralgia. The subject had applied the SOD (0.02% SOD in deoxygenated water with liposomes) topically once per day for three days but did not notice a significant decrease in pain. The subject had not applied a reduced glutathione solution to the affected area. The subject also presented symptoms indicative of an infection.

### Example 23 – Treatment of Ankylosing Spondylitis

The middle-aged male subject was diagnosed with ankylosing spondylitis 15 years previously. He was being treated with meloxicam. On bad days, his pain level was 8 or 9 on a scale from 0 (no pain) to 10 (high pain). The subject administered 6 drops of SOD solution (0.02% SOD in deoxygenated water with liposomes) onto the spot for a total of 6 drops total. After 10-15 minutes, the subject noticed that the area had reduced in pain, but the area of pain had moved to other portions of this body. The subject applied the SOD solution to each portion of his body as the pain moved. After one week of treatment, the subject observed that the SOD solution had a more pronounced effect when the pain was closer to the surface of the skin rather than in a deeper area.

### Example 24 – Treatment of Edema

The subject was a woman in her 20s who had been in a car accident a few hours earlier in which her left knee had hit the dashboard. X-rays did not show any damage, but there was swelling in her left knee. The subject also had pain and burning in her shoulders and neck. She was at a pain level of 7 on a scale from 0 (no pain) to 10 (high pain). The subject applied the topical SOD solution (0.02% SOD in deoxygenated water with liposomes) to the front of her left knee. The pain was not noticeably different after about 30 seconds. The subject topically applied the SOD solution to the back of her left knee. The subject topically applied the SOD solution to her neck and shoulders. After about 3 minutes, the burning and pain in the knee had subsided completely, and the burning and pain in her neck and shoulders was also completely gone.

### Example 25 – Treatment of Eczema

The subject was a female in her early 20s who had moderate to severe eczema that was recurrent over the previous two years. The subject topically applied the SOD (0.02% SOD in deoxygenated water with liposomes) to the affected area on her legs, and applied lotion afterwards. After about 72 hours, the eczema had cleared up about halfway with reduced irritation.

### Example 26 – Treatment of Erectile Dysfunction

Three male subjects with a history of erectile dysfunction (ED) were directed to apply the SOD solution (0.02% SOD in deoxygenated water with liposomes) to their entire groin area. All 3 subjects, within 3-5 minutes of application, had complete resolution of their ED symptoms – reduced time to erection, increased length of erection, increased sexual desire.

It is understood that the above-described various types of compositions, dosage forms and/or modes of applications are only illustrative of preferred embodiments of the present disclosure. Numerous modifications and alternative arrangements may be devised by those skilled in the art without departing from the spirit and scope of the present disclosure and the appended claims are intended to cover such modifications and arrangements. Thus, while the present disclosure has been described above with particularity and detail in connection with what is presently deemed to be the most practical and preferred embodiments of the disclosure, it will be apparent to those of ordinary skill in the art that variations including, but not limited to, variations in size, materials, shape, form, function and manner of operation, assembly and use may be made without departing from the principles and concepts set forth herein.

What is claimed is:

1. A topical composition, comprising:
a combination of a therapeutically effective amount of superoxide dismutase (SOD) with a stabilizing carrier that is suitable for topical administration.

2. The topical composition of claim 1, wherein the stabilizing carrier comprises deoxygenated water.

3. The topical composition of claim 2, wherein the deoxygenated water has less than one or more of: about 10 ppm of oxygen, about 5 ppm of oxygen, about 3 ppm of oxygen, about 1 ppm of oxygen, or combinations thereof.

4. The topical composition of claim 1, wherein the topical composition further comprises a support agent selected from the group consisting of: a stabilizing agent, a preservative, an emollient, an adjuvant, and combinations thereof.

5. The topical composition of claim 4, wherein the support agent is a stabilizing agent.

6. The topical composition of claim 5, wherein the stabilizing agent includes an oxygen scavenger, a lipid, a synthetic polymer, a natural polymer, a polyelectrolyte, a protein, an amino acid, a co-polymer, an emulsion, a gel, an inert gas, or a combination thereof.

7. The topical composition of claim 5, wherein the stabilizing agent is an encapsulating agent that forms a nanoemulsion, a liposome, a nanogel, or a combination thereof.

8. The topical composition of claim 7, wherein the encapsulating agent is selected from the group consisting essentially of: chitosan, cyclodextrin, dextran, starch, silicon, tragacanth, and combinations thereof.

9. The topical composition of claim 5, wherein the stabilizing agent forms a stabilizing complex with the SOD.

10. The topical composition of claim 5, wherein the stabilizing agent is a lipid selected from the group consisting essentially of: phospholipids, glycolipids, cholesterol, triglycerides, fatty acids, fatty acid glycerides, surfactants, or a combination thereof.

11. The topical composition of claim 10, wherein the lipid forms a liposome, an inverted micelle, or a combination thereof.

12. The topical composition of claim 1, wherein the topical composition further comprises an additional active agent.

13. The topical composition of claim 12, wherein the additional active agent is a member selected from the group consisting of: a catalase, an antioxidant, an anti-infective agent, an antibiotic, an anti-tumor agent, an anti-inflammatory agent, an anesthetic, an analgesic, an anti-rheumatic agent, a growth factor, a cytokine, an amino acid, a protein, a vaccine, a hormone, a vitamin, and combinations thereof.

14. The topical composition of claim 12, wherein the additional active agent is an anesthetic.

15. The topical composition of claim 14, wherein the anesthetic is a member selected from the group consisting of: articaine, bupivacaine, cinchocaine, etidocaine, levobupivacaine, lidocaine, mepivacaine, prilocaine, ropivacaine, trimecaine, and combinations thereof.

16. The topical composition of claim 14, wherein the anesthetic is lidocaine.

17. The topical composition of claim 12, wherein the additional active agent is in the same or a different composition as the SOD.

18. The topical composition of claim 12, wherein the additional active agent is present at a concentration of from about 0.0001 wt% to about 10 wt%.

19. The topical composition of claim 1, wherein the topical composition has a pH of from about 6.8 to about 7.4.

20. The topical composition of claim 1, wherein the topical composition has a viscosity of from about 1 cP to about 1000 cP.

21. The topical composition of claim 1, wherein the topical composition is formulated as one of: a solution, a suspension, an emulsion, a gel, a hydrogel, a thermo-responsive gel, a cream, an ointment, a paste, an adhesive, a liquid reservoir, a patch, or a combination thereof.

22. The topical composition of claim 1, wherein the SOD is present in the topical composition at a concentration of from about 0.0001 wt% to about 10 wt%.

23. The topical composition of claim 1, wherein when the SOD is present in the topical composition at a concentration of 0.02 wt%:
more than about 80% of the SOD remains in a reactive form after a period of about 2 weeks when stored at room temperature and 75% relative humidity; or
more than about 80% of the SOD remains in a reactive form after a period of about 2 weeks when stored at 40° C. and 75% relative humidity; or
more than about 80% of the SOD remains in a reactive form after a period of about 4 weeks when stored at room temperature and 75% relative humidity; or
more than about 80% of the SOD remains in a reactive form after a period of about 4 weeks when stored at 40° C. and 75% relative humidity.

24. The topical composition of claim 1, wherein the topical composition consists essentially of SOD and deoxygenated water.

25. The topical composition of claim 1, wherein the topical composition is substantially free of: ethanol, capsaicin, menthol, carbomer, and combinations thereof.

26. The topical composition of claim 1, wherein the SOD comprises human recombinant SOD (rhSOD), SOD-1, SOD-2, SOD-3, or a combination thereof.

27. A method of treating a condition in a subject that is responsive to treatment with superoxide dismutase (SOD), comprising:
administering a therapeutically effective amount of the topical composition of claim 1 to the subject.

28. The method of claim 27, wherein the condition is one or more of pain, edema, inflammation, erectile dysfunction, or interstitial cystitis.

29. The method of claim 28, wherein the pain is one or more of chronic pain, back pain, leg pain, ankle pain, j oint pain, arthritic pain, wound pain, or combinations thereof.

30. The method of claim 27, wherein the treatment provides a reduction in symptoms of at least 10% within a selected amount of time after administration.

31. The method of claim 27, wherein the topical composition comprises a dosage form having from about 25 ul to about 10 ml of SOD.

32. The method of claim 27, further comprising administering the therapeutically effective amount of the topical composition to the subject 1 to 5 times per day.

33. The method of claim 27, further comprising administering the therapeutically effective amount of the topical composition to the subject according to a dosage regimen of at least once per day for a duration of from about a single day to about 3 months.
